(19) United States
(12) Patent Application Publication
(10) Pub. No.: US 2011/0281869 A1
(43) Pub. Date: Nov. 17, 2011

(54) TETRAHYDROISOQUINOLINES AS ANTIMALARIAL AGENTS

(76) Inventors: Hamed Aissaoui, Pulversheim (FR); Christoph Boss, Allschwil (CH); Olivier Corminboeuf, Allschwil (CH); Marie-Celine Frantz, Pittsburgh, PA (US)

(21) Appl. No.: 12/993,030

(22) PCT Filed: May 18, 2009
(86) PCT No.: PCT/IB2009/052050

§ 371 Date: Nov. 16, 2010

(30) Foreign Application Priority Data
May 19, 2008 (IB) PCT IB2008 051956

(51) Int. Cl. A61K 31/4725; C07D 401/14; C07D 413/14; A61P 33/06; A61K 31/5377; A61K 31/497; A61K 31/501; A61P 33/02; C07D 401/12; A61K 31/506

(52) U.S. Cl. 514/235.2; 546/146; 544/328; 544/128; 544/333; 544/238; 544/405; 544/295; 514/307; 514/256; 514/252.02; 514/255.05; 514/252.04

(57) ABSTRACT

The invention relates to novel tetrahydroisoquinoline derivatives and their use as active ingredients in the preparation of pharmaceutical compositions. The invention also concerns related aspects including pharmaceutical compositions containing one or more of those compounds and their use as medicaments for the treatment or prevention of protozoal infections, such as especially malaria.

The invention relates to novel compounds of the formula I. The invention also concerns related aspects including processes for the preparation of the compounds, pharmaceutical compositions containing one or more compounds of the formula I and especially their use as medicaments to treat or prevent malaria infections or to treat or prevent other protozoal diseases like sleeping sickness, Chagas disease, amebiasis, giardiasis, trichomoniasis, toxoplasmosis, and leishmaniasis.

### BACKGROUND OF THE INVENTION

Numerous serious diseases affecting humans as well as domestic and livestock animal are caused by protozoal organisms such as kinetoplastida, apicomplexa, anaerobic protozoa, microsporidia and plasmodium, for example. The clinically most relevant of these diseases is malaria.

Malaria is one of the most serious and complex health problems affecting humanity in the 21^{st} century. The disease affects about 300 million people worldwide, killing 1 to 1.5 million people every year. Malaria is an infectious disease caused by four species of the protozoan parasite plasmodium, P. falciparum being the most severe of the four. All attempts to develop vaccines against P. falciparum have failed so far. Therefore, therapies and preventive measures against malaria are confined to drugs. Various classes of antimalarial drugs exist. The most widely used are the quinoline antimalarials, e.g. chloroquine which has been an especially effective drug for both prophylaxis and therapy. However, resistance to many of the currently available antimalarial drugs is spreading rapidly, threatening people in areas where malaria is endemic. Reports of multi-drug resistant strains of malaria parasites render the search for new antimalarial agents especially urgent.

P. falciparum enters the human body by way of bites of the female anophelino mosquito (it may also be transmitted by blood transfusion from asymptotic donors; almost all infected blood components including red cells, platelet concentrates, white cells, cryoprecipitates and fresh plasma can transmit malaria). The plasmodium parasite initially populates the liver, and during later stages of the infectious cycle reproduces in red blood cells. During this stage, the parasite degrades hemoglobin and uses the degradation products as nutrients for growth.

The limitations of the current antiprotozoal chemotherapeutic arsenal underscore the need for new drugs in this therapeutic area. The present invention relates to the identification of novel low molecular weight, non-peptidic, non-quinoline compounds of formula I which are useful in the treatment and/or prevention of protozoal infections, especially in the treatment and/or prevention of malaria, in particular plasmodium falciparum malaria.

### DETAILED DESCRIPTION OF THE INVENTION

(i) The present invention relates to novel compounds of the formula I:

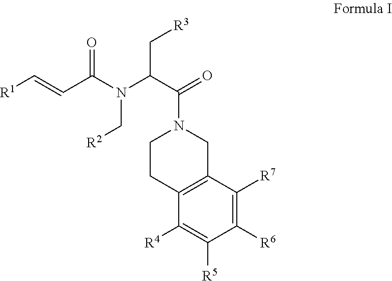

wherein

R¹ represents aryl or heteroaryl, wherein these two radicals can optionally be mono-, di-, tri-, or tetra-substituted, wherein the substituents are independently selected from the group consisting of halogen, (C₁-C₄)alkyl, (C₁-C₄)alkoxy, cycloalkyl, trifluoromethyl, trifluoromethoxy, and amino, wherein the amino group is optionally mono- or di-substituted with (C₁-C₄)alkyl or mono-substituted with (C₁-C₄)alkyl-carbonyl; or R¹ represents aryl wherein two adjacent carbon ring atoms of the aryl moiety are substituted with (C₁-C₂)alkylenedioxy, wherein the (C₁-C₂)alkylene moiety is optionally mono- or di-substituted, wherein the substituents are independently selected from the group consisting of halogen and (C₁-C₄)alkyl;

R² represents aryl or heteroaryl, wherein these two radicals can optionally be mono-, di-, tri-, or tetra-substituted, wherein the substituents are independently selected from the group consisting of halogen; (C₁-C₄)alkyl; (C₁-C₄)alkoxy; cycloalkyl; trifluoromethyl; trifluoromethoxy; heterocycloalkyl, that can optionally be mono-substituted on one nitrogen ring atom, if present, with (C₁-C₄)alkyl or (C₁-C₄)alkyl-carbonyl; aryl or heteroaryl, wherein these two radicals can optionally be mono-, di-, tri-, or tetra-substituted, wherein the substituents are independently selected from the group consisting of halogen, (C₁-C₄)alkyl, (C₁-C₄)alkoxy, cycloalkyl, trifluoromethyl, and trifluoromethoxy; 2-methyl-2H-tetrazol-5-yl; and 1-oxy-pyridin-4-yl;

R³ represents aryl or heteroaryl, wherein these two radicals can optionally be mono-, di-, tri-, or tetra-substituted, wherein the substituents are independently selected from the group consisting of halogen, (C₁-C₄)alkyl, (C₁-C₄)alkoxy, cycloalkyl, trifluoromethyl, and trifluoromethoxy; or R³ represents heterocycloalkyl that can optionally be mono-substituted on one nitrogen ring atom, if present, with (C₁-C₄)alkyl or (C₁-C₄)alkyl-carbonyl; and

R⁴, R⁵, R⁶, and R⁷ independently represent hydrogen, halogen, (C₁-C₄)alkyl, (C₁-C₄)alkoxy, cycloalkyl, or trifluoromethyl.

The general terms used hereinbefore and hereinafter preferably have, within this disclosure, the following meanings, unless otherwise indicated:

The term (C₁-C₄)alkyl, alone or in combination with other groups, means saturated, straight or branched chain groups with one to four carbon atoms, preferably one to three carbon atoms, i.e. methyl, ethyl, n-propyl, iso-propyl, n-butyl, iso-butyl, sec-butyl, and tert-butyl. The methyl, ethyl and isopropyl groups are preferred.

The term (C₁-C₄)alkoxy, alone or in combination with other groups, refers to an R—O-group, wherein R is a (C₁-C₄)alkyl, i.e. methoxy, ethoxy, n-propoxy, iso-propoxy, n-butoxy, iso-butoxy, sec-butoxy, and tert-butoxy. The methoxy group is a preferred group.

The term (C₁-C₂)alkylenedioxy refers to methylenedioxy and 1,2-ethylenedioxy. If R¹ represents aryl wherein two adjacent carbon ring atoms of the aryl moiety are substituted with (C₁-C₂)alkylenedioxy, this means that methylenedioxy or 1,2-ethylenedioxy is attached via its oxygen atoms to the two adjacent carbon ring atoms of the aryl moiety, to form, together with the two adjacent carbon ring atoms, a 5- or 6-membered ring, respectively.

The term halogen means fluorine, chlorine, bromine or iodine, preferably fluorine or chlorine.

The term cycloalkyl, alone or in combination with other groups, means a saturated cyclic hydrocarbon ring system with 3 to 7 carbon atoms, i.e. cyclopropyl, cyclobutyl, cyclopentyl, cyclohexyl, and cycloheptyl. The cyclopropyl group is a preferred group.

The term aryl, alone or in combination with other groups, relates to a phenyl or naphthyl group, preferably a phenyl group.

The term heteroaryl, alone or in combination with other groups, means a 5- to 10-membered monocyclic or bicyclic aromatic ring containing 1, 2, or 3 ring heteroatoms independently selected from oxygen, nitrogen, and sulfur. Examples of such heteroaryl groups are furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidyl, pyridazinyl, pyrazinyl, indolyl, isoindolyl, benzofuranyl, isobenzofuranyl, benzothiophenyl, indazolyl, benzimidazolyl, benzoxazolyl, benzoisoxazolyl, benzothiazolyl, benzotriazolyl, benzoxadiazolyl, benzothiadiazolyl, quinolinyl, isoquinolinyl, naphthyridinyl, cinnolinyl, quinazolinyl, quinoxalinyl, and phthalazinyl. In one embodiment, the term heteroaryl refers to the group selected from oxazolyl, isoxazolyl, thiazolyl, thiadiazolyl, imidazolyl, pyrazolyl, pyridyl, and pyrimidyl, such as especially oxazolyl, isoxazolyl, thiazolyl, imidazolyl, pyrazolyl, pyridyl, and pyrimidyl. In a further embodiment the term heteroaryl refers to pyrazolyl.

The term heterocycloalkyl, alone or in combination with other groups, means a 4-, 5-, or 6-membered saturated cyclic hydrocarbon ring system containing 1, 2, or 3 ring heteroatoms independently selected from oxygen, nitrogen, and sulfur. Examples of such heterocycloalkyl groups are pyrrolidinyl, piperidyl, morpholinyl, and piperazinyl.

ii) A further embodiment of the invention relates to compounds of the formula I according to embodiment i), wherein R² represents aryl or heteroaryl, wherein these two radicals can optionally be mono-, di-, tri-, or tetra-substituted, wherein the substituents are independently selected from the group consisting of halogen; (C₁-C₄)alkyl; (C₁-C₄)alkoxy; cycloalkyl; trifluoromethyl; trifluoromethoxy; heterocycloalkyl, that can optionally be mono-substituted on one nitrogen ring atom, if present, with (C₁-C₄)alkyl or (C₁-C₄)alkyl-carbonyl; and aryl or heteroaryl, wherein these two radicals can optionally be mono-, di-, tri-, or tetra-substituted, wherein the substituents are independently selected from the group consisting of halogen, (C₁-C₄)alkyl, (C₁-C₄)alkoxy, cycloalkyl, trifluoromethyl, and trifluoromethoxy.

iii) A further embodiment of the invention relates to compounds of the formula I according to embodiment i) or ii), wherein the carbon atom to which —CH₂—R³ is attached is in the (S)-configuration:

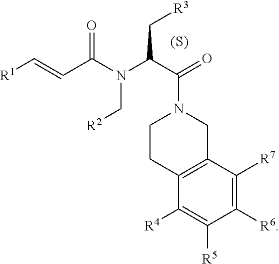

iv) A further embodiment of the invention relates to compounds of the formula I according to any one of embodiments i) to iii), wherein

R¹ represents mono-substituted aryl or mono-substituted heteroaryl, wherein the substituent is selected from the group consisting of (C₁-C₄)alkyl, (C₁-C₄)alkoxy, cycloalkyl, trifluoromethyl, and trifluoromethoxy.

v) A further embodiment of the invention relates to compounds of the formula I according to embodiment iv), wherein R¹ represents mono-substituted aryl or mono-substituted heteroaryl, such as especially mono-substituted phenyl, pyridyl or pyrimidyl, wherein the substituent is selected from the group consisting of methyl, methoxy, and trifluoromethyl.

vi) A further embodiment of the invention relates to compounds of the formula I according to any one of embodiments i) to iii), wherein

R¹ represents phenyl, pyridyl or pyrimidyl, wherein these three radicals are mono-, di-, or tri-substituted (preferably mono-substituted), wherein the substituents are independently selected from the group consisting of halogen, (C₁-C₄)alkyl such as methyl, (C₁-C₄)alkoxy such as methoxy, trifluoromethyl, and amino, wherein the amino group is optionally mono- or di-substituted with (C₁-C₄)alkyl such as methyl; or R¹ represents pyrazolyl, imidazolyl, thiazolyl, isoxazolyl, oxazolyl, thiadiazolyl, or pyridazinyl, wherein these radicals are mono-, di-, or tri-substituted, wherein the substituents are independently selected from the group consisting of (C₁-C₄)alkyl such as methyl and (C₁-C₄)alkoxy such as methoxy, such R¹ radicals being preferably 1-methyl-1H-pyrazol-3-yl, 2,5-dimethyl-2H-pyrazol-3-yl, 1,3,5-trimethyl-1H-pyrazol-4-yl, 2,3-dimethyl-3H-imidazol-4-yl, 2,4-dimethyl-thiazol-5-yl, 2-methyl-thiazol-4-yl, 5-methyl-isoxazol-3-yl, 3,5-dimethyl-isoxazol-4-yl, 2,5-dimethyl-oxazol-4-yl, [1,2,3]thiadiazol-4-yl, 1,5-dimethyl-1H-pyrazol-4-yl, 6-methoxy-pyridazin-3-yl, 1-methyl-1H-pyrazol-4-yl, 4-methyl-thiazol-5-yl, or 2-methyl-thiazol-5-yl; or R¹ represents phenyl, wherein two adjacent carbon ring atoms of the phenyl moiety are substituted with (C₁-C₂)alkylenedioxy, such as benzo[1,3]dioxol-5-yl.

vii) A further embodiment of the invention relates to compounds of the formula I according to any one of embodiments i) to vi), wherein

R² represents mono-substituted aryl or mono-substituted heteroaryl, wherein the substituent is selected from the group consisting of halogen, (C₁-C₄)alkyl, (C₁-C₄)alkoxy, cycloalkyl, trifluoromethyl, trifluoromethoxy, aryl, heteroaryl, and heterocycloalkyl wherein the heterocycloalkyl can optionally be mono-substituted on one nitrogen ring atom, if present, with (C₁-C₄)alkyl or (C₁-C₄)alkyl-carbonyl.

viii) A further embodiment of the invention relates to compounds of the formula I according to any one of embodiments i) to yl), wherein

R² represents phenyl or pyridyl, wherein these two radicals can optionally be mono-substituted (especially in para-position) with a substituent selected from the group consisting of (C₁-C₄)alkyl; morpholinyl; piperazinyl mono-substituted on one nitrogen ring atom with (C₁-C₄)alkyl; pyridyl; pyrimidyl; pyrazinyl; pyridazinyl; triazolyl; pyrazolyl; thiazolyl; oxazolyl; 2-methyl-2H-tetrazol-5-yl; and 1-oxy-pyridin-4-yl. Preferably the substituent is selected from the group consisting of (C₁-C₄)alkyl such as ethyl, morpholinyl such as morpholin-4-yl, 4-methyl-piperazin-1-yl, pyridyl such as pyridin-2-yl or pyridin-4-yl, pyrimidyl such as pyrimidin-5-yl or pyrimidin-2-yl, pyrazin-2-yl, pyridazin-3-yl, [1,2,3]triazol-1-yl, [1,2,4]triazol-1-yl, pyrazol-1-yl, thiazol-2-yl, thiazol-5-yl, oxazol-5-yl, 2-methyl-2H-tetrazol-5-yl, and 1-oxy-pyridin-4-yl.

ix) A further embodiment of the invention relates to compounds of the formula I according to any one of embodiments i) to viii), wherein

R³ represents phenyl.

x) A further embodiment of the invention relates to compounds of the formula I according to any one of embodiments i) to ix), wherein R⁴, R⁵, R⁶, and R⁷ all represent hydrogen.

xi) In another embodiment, the present invention relates to compounds of the formula I according to embodiment i) or ii), wherein

R¹ represents phenyl, pyridyl or pyrimidyl, wherein these three radicals are mono-, di-, or tri-substituted, wherein the substituents are independently selected from the group consisting of halogen, (C₁-C₄)alkyl such as especially methyl, (C₁-C₄)alkoxy such as especially methoxy, trifluoromethyl, and amino, wherein the amino group is optionally mono- or di-substituted with (C₁-C₄)alkyl such as especially methyl; or R¹ represents 1-methyl-1H-pyrazol-3-yl, 2,5-dimethyl-2H-pyrazol-3-yl, 1,3,5-trimethyl-1H-pyrazol-4-yl, 2,3-dimethyl-3H-imidazol-4-yl, 2,4-dimethyl-thiazol-5-yl, 2-methyl-thiazol-4-yl, 5-methyl-isoxazol-3-yl, 3,5-dimethyl-isoxazol-4-yl, 2,5-dimethyl-oxazol-4-yl, [1,2,3]thiadiazol-4-yl, or 1,5-dimethyl-1H-pyrazol-4-yl; or R¹ represents phenyl, wherein two adjacent carbon ring atoms of the phenyl moiety are substituted with (C₁-C₂)alkylenedioxy, such as especially benzo[1,3]dioxol-5-yl;

R² represents phenyl or pyridyl, wherein these two radicals can optionally be mono-substituted (especially in para-position) with (C₁-C₄)alkyl such as especially ethyl, morpholinyl such as especially morpholin-4-yl, pyridyl such as especially pyridin-2-yl or pyridin-4-yl, or pyrimidyl such as especially pyrimidin-5-yl;

R³ represents phenyl or pyridyl, such as especially phenyl; and

R⁴, R⁵, R⁶, and R⁷ independently represent hydrogen, halogen, (C₁-C₄)alkoxy such as especially methoxy and ethoxy, or trifluoromethyl.

xii) In another embodiment, the present invention relates to compounds of the formula I according to embodiment i), wherein

R¹ represents phenyl, pyridyl or pyrimidyl, wherein these three radicals are mono-, di-, or tri-substituted, wherein the substituents are independently selected from the group consisting of halogen, (C₁-C₄)alkyl such as methyl, (C₁-C₄)alkoxy such as methoxy, trifluoromethyl, and amino, wherein the amino group is optionally mono- or di-substituted with (C₁-C₄)alkyl such as methyl; or R¹ represents 1-methyl-1H-pyrazol-3-yl, 2,5-dimethyl-2H-pyrazol-3-yl, 1,3,5-trimethyl-1H-pyrazol-4-yl, 2,3-dimethyl-3H-imidazol-4-yl, 2,4-dimethyl-thiazol-5-yl, 2-methyl-thiazol-4-yl, 5-methyl-isoxazol-3-yl, 3,5-dimethyl-isoxazol-4-yl, 2,5-dimethyl-oxazol-4-yl, [1,2,3]thiadiazol-4-yl, 1,5-dimethyl-1H-pyrazol-4-yl, 6-methoxy-pyridazin-3-yl, 1-methyl-1H-pyrazol-4-yl, 4-methyl-thiazol-5-yl, or 2-methyl-thiazol-5-yl; or

R¹ represents phenyl, wherein two adjacent carbon ring atoms of the phenyl moiety are substituted with (C₁-C₂)alkylenedioxy, such as benzo[1,3]dioxol-5-yl;

R² represents phenyl or pyridyl, wherein these two radicals can optionally be mono-substituted (especially in para-position) with a substituent selected from the group consisting of (C₁-C₄)alkyl such as ethyl, morpholinyl such as morpholin-4-yl, 4-methyl-piperazin-1-yl, pyridyl such as pyridin-2-yl or pyridin-4-yl, pyrimidyl such as pyrimidin-5-yl or pyrimidin-2-yl, pyrazin-2-yl, pyridazin-3-yl, [1,2,3]triazol-1-yl, [1,2,4]triazol-1-yl, pyrazol-1-yl, thiazol-2-yl, thiazol-5-yl, oxazol-5-yl, 2-methyl-2H-tetrazol-5-yl, and 1-oxy-pyridin-4-yl;

R³ represents phenyl, pyridyl such as 2-pyridyl, pyrimidyl such as pyrimidin-2-yl, isoxazolyl such as isoxazol-3-yl, or methyl-pyrazolyl such as 1-methyl-1H-pyrazol-4-yl, 1-methyl-1H-pyrazol-3-yl, or 2-methyl-2H-pyrazol-3-yl; in particular R³ respresents phenyl; and

R⁴, R⁵, R⁶, and R⁷ independently represent hydrogen, halogen, (C₁-C₄)alkoxy such as methoxy and ethoxy, or trifluoromethyl.

The compounds of formula I may contain one or more stereogenic or asymmetric centers, such as one or more asymmetric carbon atoms. The compounds of formula I may thus be present as mixtures of stereoisomers or preferably as pure stereoisomers. Mixtures of stereoisomers may be separated in a manner known to a person skilled in the art.

Where the plural form is used for compounds, salts, pharmaceutical compositions, diseases and the like, this is intended to mean also a single compound, salt, or the like.

Any reference hereinbefore or hereinafter to a compound of formula I is to be understood as referring also to salts, especially pharmaceutically acceptable salts, of a compound of formula I, as appropriate and expedient.

The term “pharmaceutically acceptable salts” refers to non-toxic, inorganic or organic acid and/or base addition salts. Reference can be made to “Salt selection for basic drugs”, Int. J. Pharm. (1986), 33, 201-217.

Examples of preferred compounds of formula I are selected from the group consisting of:
(S)—N-[1-Benzyl-2-(3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-N-(4-pyridin-2-yl-benzyl)-3-(6-trifluoromethyl-pyridin-3-yl)-acrylamide;
(S)—N-[1-Benzyl-2-(3,4-dihydro-1H-isoquinolin-2-yl)-2-oxo-ethyl]-N-(4-pyrimidin-5-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-N-(4-pyrimidin-5-yl-benzyl)-3-(6-trifluoromethyl-pyridin-3-yl)-acrylamide;
(S)—N-[1-Benzyl-2-(3,4-dihydro-1H-isoquinolin-2-yl)-2-oxo-ethyl]-N-(6-morpholin-4-yl-pyridin-3-ylmethyl)-3-(4-trifluoromethyl-phenyl)-acrylamide;
(S)—N-[1-Benzyl-2-(3,4-dihydro-1H-isoquinolin-2-yl)-2-oxo-ethyl]-N-pyridin-2-ylmethyl-3-(4-trifluoromethyl-phenyl)-acrylamide;
(S)—N-Benzyl-N-[1-benzyl-2-(3,4-dihydro-1H-isoquinolin-2-yl)-2-oxo-ethyl]-3-(4-trifluoromethyl-phenyl)-acrylamide;
(S)—N-[1-Benzyl-2-(3,4-dihydro-1H-isoquinolin-2-yl)-2-oxo-ethyl]-3-(4-methoxy-phenyl)-N-(6-morpholin-4-yl-pyridin-3-ylmethyl)-acrylamide;
(S)—N-[1-Benzyl-2-(6,7-ethoxy-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(6-fluoro-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(3,4-dihydro-1-H-isoquinolin-2-yl)-2-oxo-ethyl]-3-(4-methoxy-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-N-(4-pyridin-2-yl-benzyl)-3-(3-trifluoromethyl-phenyl)-acrylamide;
(S)—N-[1-Benzyl-2-(3,4-dihydro-1-H-isoquinolin-2-yl)-2-oxo-ethyl]-3-(4-bromo-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(3,5-dimethoxy-phenyl)-N-(4-pyridin-2-yl-benzyl)-acrylamide;
(S)—N-Benzyl-N-[1-benzyl-2-(3,4-dihydro-1H-isoquinolin-2-yl)-2-oxo-ethyl]-3-(6-trifluoromethyl-pyridin-3-yl)-acrylamide;
(S)—N-[1-Benzyl-2-(6-fluoro-3,4-dihydro-1H-isoquinolin-2-yl)-2-oxo-ethyl]-N-(4-pyridin-2-yl-benzyl)-3-(6-trifluoromethyl-pyridin-3-yl)-acrylamide;
(S)—N-[1-Benzyl-2-(3,4-dihydro-1H-isoquinolin-2-yl)-2-oxo-ethyl]-N-(6-morpholin-4-yl-pyridin-3-ylmethyl)-3-p-tolyl-acrylamide;
(S)—N-[1-Benzyl-2-(3,4-dihydro-1-H-isoquinolin-2-yl)-2-oxo-ethyl]-3-(3-methoxy-phenyl)-N-(4-pyridin-2-yl-benzyl)-acrylamide;
(S)-3-Benzo[1,3]dioxol-5-yl-N-[benzyl-2-(3,4-dihydro-1H-isoquinolin-2-yl)-2-oxo-ethyl]-N-(4-pyridin-2-yl-benzyl)-acrylamide;
(S)—N-[1-Benzyl-2-(3,4-dihydro-1-H-isoquinolin-2-yl)-2-oxo-ethyl]-3-(4-fluoro-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(2,4-difluoro-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(2-bromo-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(3,4-difluoro-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(3-bromo-phenyl)-N-(4-pyridin-2-yl-benzyl)acrylamide;
(S)—N-[1-Benzyl-2-(3,4-dihydro-1-H-isoquinolin-2-yl)-2-oxo-ethyl]-3-(2,5-dimethyl-phenyl)-N-(4-pyridin-2-yl-benzyl)-acrylamide;
(S)—N-Benzyl-N-[1-Benzyl-2-(3,4-dihydro-1-H-isoquinolin-2-yl)-2-oxo-ethyl]-3-p-tolyl-acrylamide;
(S)—N-[1-Benzyl-2-(3,4-dihydro-1H-isoquinolin-2-yl)-2-oxo-ethyl]-N-(4-ethyl-benzyl)-3-p-tolyl-acrylamide;
(S)—N-[1-Benzyl-2-(3,4-dihydro-1-H-isoquinolin-2-yl)-2-oxo-ethyl]-3-(2,3-dichloro-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(2-chloro-phenyl)-N-(4-pyridin-2-yl-benzyl)acrylamide;
(S)—N-[1-Benzyl-2-(3,4-dihydro-1-H-isoquinolin-2-yl)-2-oxo-ethyl]-3-(3-fluoro-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(2,5-dimethoxy-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(3,4,5-trimethoxy-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-N-(4-pyridin-2-yl-benzyl)-3-o-tolyl-acrylamide;
(S)—N-[1-Benzyl-2-(3,4-dihydro-1-H-isoquinolin-2-yl)-2-oxo-ethyl]-N-(4-pyridin-2-yl-benzyl)-3-m-tolyl-acrylamide;
(S)—N-[1-Benzyl-2-(3,4-dihydro-1-H-isoquinolin-2-yl)-2-oxo-ethyl]-N-(4-pyridin-2-yl-benzyl)-3-p-tolyl-acrylamide;
(S)—N-[1-Benzyl-2-(3,4-dihydro-1-H-isoquinolin-2-yl)-2-oxo-ethyl]-3-(2,4-dichloro-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(2-methoxy-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(4-chloro-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(3-chloro-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(4-dimethylamino-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-N-(4-pyridin-4-yl-benzyl)-3-(4-trifluoromethyl-phenyl)-acrylamide;
(S)—N-[2-(3,4-Dihydro-1H-isoquinolin-2-yl)-2-oxo-1-pyridin-2-ylmethyl-ethyl]-N-(4-ethyl-benzyl)-3-p-tolyl-acrylamide;
(S)—N-[1-Benzyl-2-(3,4-dihydro-1H-isoquinolin-2-yl)-2-oxo-ethyl]-N-pyridin-3-ylmethyl-3-p-tolyl-acrylamide;
(S)—N-[1-Benzyl-2-(3,4-dihydro-1H-isoquinolin-2-yl)-2-oxo-ethyl]-3-(2,3,6-trifluoro-phenyl)-N-(4-pyridin-2-yl-benzyl)-acrylamide;
(S)—N-[1-Benzyl-2-(3,4-dihydro-1H-isoquinolin-2-yl)-2-oxo-ethyl]-3-(2,5-difluoro-phenyl)-N-(4-pyridin-2-yl-benzyl)-acrylamide;
(S)—N-[1-Benzyl-2-(3,4-dihydro-1H-isoquinolin-2-yl)-2-oxo-ethyl]-3-(2-trifluoromethyl-phenyl)-N-(4-pyridin-2-yl-benzyl)-acrylamide;
(S)—N-[1-Benzyl-2-(6-fluoro-3,4-dihydro-1H-isoquinolin-2-yl)-2-oxo-ethyl]-N-(4-pyridin-2-yl-benzyl)-3-(6-trifluoromethyl-pyridin-3-yl)-acrylamide;
(S)—N-Benzyl-N-[1-benzyl-2-(3,4-dihydro-1H-isoquinolin-2-yl)-2-oxo-ethyl]-3-(6-trifluoromethyl-pyridin-3-yl)-acrylamide;
(S)—N-[1-Benzyl-2-(3,4-dihydro-1H-isoquinolin-2-yl)-2-oxo-ethyl]-N-(6-morpholin-4-yl-pyridin-3-ylmethyl)-3-p-tolyl-acrylamide;
(S)—N-[1-Benzyl-2-oxo-2-(6-trifluoromethyl-3,4-dihydro-1H-isoquinolin-2-yl)-ethyl]-N-(4-pyridin-2-yl-benzyl)-3-(4-trifluoromethyl-phenyl)-acrylamide;
(S)—N-[1-Benzyl-2-oxo-2-(7-trifluoromethyl-3,4-dihydro-1H-isoquinolin-2-yl)-ethyl]-N-(4-pyridin-2-yl-benzyl)-3-(4-trifluoromethyl-phenyl)-acrylamide;
(S)—N-[1-Benzyl-2-(7-chloro-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(7-methoxy-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(7-fluoro-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(8-chloro-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(8-methoxy-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(5-chloro-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(5-fluoro-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(5-methoxy-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(6-methoxy-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(6-chloro-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-3-(1-methyl-1H-pyrazol-3-yl)-N-(4-pyridin-2-yl-benzyl)-acrylamide;
(S)—N-[1-Benzyl-2-(3,4-dihydro-1H-isoquinolin-2-yl)-2-oxo-ethyl]-3-(2,5-dimethyl-2H-pyrazol-3-yl)-N-(4-pyridin-2-yl-benzyl)-acrylamide;
(S)—N-[1-Benzyl-2-(3,4-dihydro-1H-isoquinolin-2-yl)-2-oxo-ethyl]-N-(4-pyridin-2-yl-benzyl)-3-(1,3,5-trimethyl-1H-pyrazol-4-yl)-acrylamide;
(S)—N-[1-Benzyl-2-(3,4-dihydro-1H-isoquinolin-2-yl)-2-oxo-ethyl]-3-(2,3-dimethyl-3H-imidazol-4-yl)-N-(4-pyridin-2-yl-benzyl)acrylamide;
(S)—N-[1-Benzyl-2-(3,4-dihydro-1H-isoquinolin-2-yl)-2-oxo-ethyl]-3-(2,4-dimethyl-thiazol-5-yl)-N-(4-pyridin-2-yl-benzyl)-acrylamide;
(S)—N-[1-Benzyl-2-(3,4-dihydro-1H-isoquinolin-2-yl)-2-oxo-ethyl]-3-(2-methyl-thiazol-4-yl)-N-(4-pyridin-2-yl-benzyl)-acrylamide;
(S)—N-[1-Benzyl-2-(3,4-dihydro-1H-isoquinolin-2-yl)-2-oxo-ethyl]-3-(5-methyl-isoxazol-3-yl)-N-(4-pyridin-2-yl-benzyl)-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-(4-pyridin-2-yl-benzyl)-acrylamide;
(S)—N-[1-Benzyl-2-(3,4-dihydro-1H-isoquinolin-2-yl)-2-oxo-ethyl]-3-(2,5-dimethyl-oxazol-4-yl)-N-(4-pyridin-2-yl-benzyl)-acrylamide;
(S)—N-[1-Benzyl-2-(3,4-dihydro-1H-isoquinolin-2-yl)-2-oxo-ethyl]-N-(4-pyridin-2-yl-benzyl)-3-[1,2,3]thiadiazol-4-yl-acrylamide;
(S)—N-[1-Benzyl-2-(3,4-dihydro-1H-isoquinolin-2-yl)-2-oxo-ethyl]-N-(4-pyridin-2-yl-benzyl)-3-(5-trifluoromethyl-pyridin-2-yl)-acrylamide;
(S)—N-[1-Benzyl-2-(3,4-dihydro-1H-isoquinolin-2-yl)-2-oxo-ethyl]-3-(2-methyl-pyrimidin-5-yl)-N-(4-pyridin-2-yl-benzyl)-acrylamide;
(S)—N-[1-Benzyl-2-(3,4-dihydro-1H-isoquinolin-2-yl)-2-oxo-ethyl]-3-(6-methyl-pyridin-3-yl)-N-(4-pyridin-2-yl-benzyl)-acrylamide;
(S)—N-[1-Benzyl-2-(3,4-dihydro-1H-isoquinolin-2-yl)-2-oxo-ethyl]-3-(6-chloro-pyridin-3-yl)-N-(4-pyridin-2-yl-benzyl)-acrylamide;
(S)—N-[1-Benzyl-2-(3,4-dihydro-1H-isoquinolin-2-yl)-2-oxo-ethyl]-3-(5-methyl-pyridin-2-yl)-N-(4-pyridin-2-yl-benzyl)-acrylamide;
(S)—N-[1-Benzyl-2-(3,4-dihydro-1H-isoquinolin-2-yl)-2-oxo-ethyl]-3-(6-methoxy-pyridin-3-yl)-N-(4-pyridin-2-yl-benzyl)-acrylamide;
(S)—N-[1-Benzyl-2-(3,4-dihydro-1H-isoquinolin-2-yl)-2-oxo-ethyl]-3-(5-methoxy-pyridin-2-yl)-N-(4-pyridin-2-yl-benzyl)-acrylamide;
(S)—N-[1-Benzyl-2-(3,4-dihydro-1H-isoquinolin-2-yl)-2-oxo-ethyl]-N-(4-pyridin-2-yl-benzyl)-3-(2-trifluoromethyl-pyrimidin-5-yl)acrylamide; and
(S)—N-[1-Benzyl-2-(3,4-dihydro-1H-isoquinolin-2-yl)-2-oxo-ethyl]-3-(1,5-dimethyl-1H-pyrazol-4-yl)-N-(4-pyridin-2-yl-benzyl)-acrylamide.

Further examples of preferred compounds of formula I are selected from the group consisting of:
(S)—N-[1-Benzyl-2-(3,4-dihydro-1-H-isoquinolin-2-yl)-2-oxo-ethyl]-3-(2-fluoro-phenyl)-N-(4-pyridin-2-yl-benzyl)-acrylamide;
(S)—N-[1-Benzyl-2-(3,4-dihydro-1H-isoquinolin-2-yl)-2-oxo-ethyl]-N-(4-pyrimidin-2-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-3-(5-methyl-pyridin-2-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(6-methyl-pyridin-3-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(6-chloro-pyridin-3-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(6-methoxy-pyridin-3-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(2-methyl-pyrimidin-5-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(6-methoxy-pyridazin-3-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(2-methoxy-pyrimidin-5-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(1-methyl-1H-pyrazol-4-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(2,5-dimethyl-2H-pyrazol-3-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(1,5-dimethyl-1H-pyrazol-4-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-N-(4-pyrimidin-2-yl-benzyl)-3-(1,3,5-trimethyl-1H-pyrazol-4-yl)-acrylamide;
(S)—N-[1-Benzyl-2-(3,4-dihydro-1H-isoquinolin-2-yl)-2-oxo-ethyl]-3-(2,3-dimethyl-3H-imidazol-4-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(2,5-dimethyl-oxazol-4-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(4-methyl-thiazol-5-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(2-methyl-thiazol-4-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(2-methyl-thiazol-5-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(2,4-dimethyl-thiazol-5-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-N-(4-pyrazin-2-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-3-(5-methyl-pyridin-2-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(6-methyl-pyridin-3-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(6-chloro-pyridin-3-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(6-methoxy-pyridin-3-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(2-methyl-pyrimidin-5-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(2-methoxy-pyrimidin-5-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(6-methoxy-pyridazin-3-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(1-methyl-1H-pyrazol-4-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(2,5-dimethyl-2H-pyrazol-3-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(1,5-dimethyl-1H-pyrazol-4-yl)-N-(4-pyrazin-2-yl-benzyl)-acrylamide;
(S)—N-[1-Benzyl-2-(3,4-dihydro-1H-isoquinolin-2-yl)-2-oxo-ethyl]-N-(4-pyrazin-2-yl-benzyl)-3-(1,3,5-trimethyl-1H-pyrazol-4-yl)-acrylamide;
(S)—N-[1-Benzyl-2-(3,4-dihydro-1H-isoquinolin-2-yl)-2-oxo-ethyl]-3-(2,3-dimethyl-3H-imidazol-4-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(5-methyl-isoxazol-3-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(2,5-dimethyl-oxazol-4-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(4-methyl-thiazol-5-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(2-methyl-thiazol-4-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(2-methyl-thiazol-5-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(2,4-dimethyl-thiazol-5-yl)-N-(4-pyrazin-2-yl-benzyl)-acrylamide;
(S)—N-[1-Benzyl-2-(3,4-dihydro-1H-isoquinolin-2-yl)-2-oxo-ethyl]-N-(4-pyridazin-3-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-3-(5-methyl-pyridin-2-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(6-methyl-pyridin-3-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(6-chloro-pyridin-3-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(6-methoxy-pyridin-3-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(2-methyl-pyrimidin-5-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(2-methoxy-pyrimidin-5-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(6-methoxy-pyridazin-3-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(1-methyl-1H-pyrazol-4-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(2,5-dimethyl-2H-pyrazol-3-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(2,3-dimethyl-3H-imidazol-4-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(5-methyl-isoxazol-3-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(2,5-dimethyl-oxazol-4-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(4-methyl-thiazol-5-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(2-methyl-thiazol-4-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(2-methyl-thiazol-5-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(2,4-dimethyl-thiazol-5-yl)-N-(4-pyridazin-3-yl-benzyl)-acrylamide;
(S)—N-[1-Benzyl-2-(3,4-dihydro-1H-isoquinolin-2-yl)-2-oxo-ethyl]-N-(4-[1,2,3]triazol-1-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-3-(5-methyl-pyridin-2-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(6-methyl-pyridin-3-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(6-chloro-pyridin-3-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(6-methoxy-pyridin-3-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(2-methyl-pyrimidin-5-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(2-methoxy-pyrimidin-5-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(6-methoxy-pyridazin-3-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(1-methyl-1H-pyrazol-4-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(2,5-dimethyl-2H-pyrazol-3-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(1,5-dimethyl-1H-pyrazol-4-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-N-(4-[1,2,3]-triazol-1-yl-benzyl)-3-(1,3,5-trimethyl-1H-pyrazol-4-yl)-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(2,5-dimethyl-oxazol-4-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(4-methyl-thiazol-5-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(2-methyl-thiazol-4-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(2-methyl-thiazol-5-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(2,4-dimethyl-thiazol-5-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-N-(4-[1,2,4]triazol-1-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-3-(5-methyl-pyridin-2-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(6-methyl-pyridin-3-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(6-chloro-pyridin-3-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(6-methoxy-pyridin-3-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(2-methyl-pyrimidin-5-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(2-methoxy-pyrimidin-5-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(6-methoxy-pyridazin-3-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(1-methyl-1H-pyrazol-4-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(2,5-dimethyl-2H-pyrazol-3-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(1,5-dimethyl-1H-pyrazol-4-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-N-(4-[1,2,4]-triazol-1-yl-benzyl)-3-(1,3,5-trimethyl-1H-pyrazol-4-yl)-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(2,5-dimethyl-oxazol-4-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(4-methyl-thiazol-5-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(2-methyl-thiazol-4-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(2-methyl-thiazol-5-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(2,4-dimethyl-thiazol-5-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-N-(4-pyrazol-1-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-3-(5-methyl-pyridin-2-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(6-methyl-pyridin-3-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(6-chloro-pyridin-3-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(6-methoxy-pyridin-3-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(2-methyl-pyrimidin-5-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(2-methoxy-pyrimidin-5-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(6-methoxy-pyridazin-3-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(1-methyl-1H-pyrazol-4-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(2,5-dimethyl-2H-pyrazol-3-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(1,5-dimethyl-1H-pyrazol-4-yl)-N-(4-pyrazol-1-yl-benzyl)-acrylamide;
(S)—N-[1-Benzyl-2-(3,4-dihydro-1H-isoquinolin-2-yl)-2-oxo-ethyl]-N-(4-pyrazol-1-yl-benzyl)-3-(1,3,5-trimethyl-1H-pyrazol-4-yl)-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(2,5-dimethyl-oxazol-4-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(4-methyl-thiazol-5-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(2-methyl-thiazol-5-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(2,4-dimethyl-thiazol-5-yl)-N-(4-pyrazol-1-yl-benzyl)-acrylamide;
(S)—N-[1-Benzyl-2-(3,4-dihydro-1H-isoquinolin-2-yl)-2-oxo-ethyl]-N-(4-thiazol-2-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-N-(4-thiazol-2-yl-benzyl)-3-(1,3,5-trimethyl-1H-pyrazol-4-yl)-acrylamide;
(S)—N-[1-Benzyl-2-(3,4-dihydro-1H-isoquinolin-2-yl)-2-oxo-ethyl]-N-(4-thiazol-5-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-N-(4-thiazol-5-yl-benzyl)-3-(1,3,5-trimethyl-1H-pyrazol-4-yl)-acrylamide;
(S)—N-[1-Benzyl-2-(3,4-dihydro-1H-isoquinolin-2-yl)-2-oxo-ethyl]-N-(4-oxazol-5-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-N-(4-oxazol-5-yl-benzyl)-3-(1,3,5-trimethyl-1H-pyrazol-4-yl)-acrylamide;
(S)—N-[1-Benzyl-2-(3,4-dihydro-1H-isoquinolin-2-yl)-2-oxo-ethyl]-N-[4-(2-methyl-2H-tetrazol-5-yl)-benzyl]-3-(4-trifluoromethyl-phenyl)-acrylamide;
(S)—N-[1-Benzyl-2-(3,4-dihydro-1H-isoquinolin-2-yl)-2-oxo-ethyl]-N-[4-(2-methyl-2H-tetrazol-5-yl)-benzyl]-3-(1,3,5-trimethyl-1H-pyrazol-4-yl)-acrylamide;
(S)—N-[2-(3,4-Dihydro-1H-isoquinolin-2-yl)-2-oxo-1-pyridin-2-ylmethyl-ethyl]-3-(4-methoxy-phenyl)-N-(6-morpholin-4-yl-pyridin-3-ylmethyl)-acrylamide;
(S)—N-[1-Benzyl-2-(3,4-dihydro-1H-isoquinolin-2-yl)-2-oxo-ethyl]-N-[4-(4-methyl-piperazin-1-yl)-benzyl]-3-(2-methyl-thiazol-4-yl)-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-[4-(4-methyl-piperazin-1-yl)-benzyl]-acrylamide;
(S)—N-[1-Benzyl-2-(3,4-dihydro-1H-isoquinolin-2-yl)-2-oxo-ethyl]-3-(5-chloro-pyridin-2-yl)-N-[4-(4-methyl-piperazin-1-yl)-benzyl]-acrylamide;
(S)—N-[1-Benzyl-2-(3,4-dihydro-1H-isoquinolin-2-yl)-2-oxo-ethyl]-N-[4-(4-methyl-piperazin-1-yl)-benzyl]-3-(5-trifluoromethyl-pyridin-2-yl)-acrylamide;
(S)—N-[1-Benzyl-2-(3,4-dihydro-1H-isoquinolin-2-yl)-2-oxo-ethyl]-3-(6-methoxy-pyridin-3-yl)-N-[4-(4-methyl-piperazin-1-yl)-benzyl]acrylamide;
(S)—N-[1-Benzyl-2-(3,4-dihydro-1H-isoquinolin-2-yl)-2-oxo-ethyl]-N-[4-(4-methyl-piperazin-1-yl)-benzyl]-3-(1,3,5-trimethyl-1H-pyrazol-4-yl)-acrylamide;
(S)—N-[1-Benzyl-2-(3,4-dihydro-1H-isoquinolin-2-yl)-2-oxo-ethyl]-3-(1,5-dimethyl-1H-pyrazol-4-yl)-N-[4-(4-methyl-piperazin-1-yl)-benzyl]acrylamide;
(S)—N-[1-Benzyl-2-(3,4-dihydro-1H-isoquinolin-2-yl)-2-oxo-ethyl]-3-(1-methyl-1H-pyrazol-3-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(5-methyl-isoxazol-3-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(5-methyl-pyridin-2-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(6-methyl-pyridin-3-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(6-chloro-pyridin-3-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(6-methoxy-pyridin-3-yl)-N-(4-morpholin-4-yl-benzyl)-acrylamide;
(S)—N-[1-Benzyl-2-(3,4-dihydro-1H-isoquinolin-2-yl)-2-oxo-ethyl]-N-(4-morpholin-4-yl-benzyl)-3-(5-trifluoromethyl-pyridin-2-yl)-acrylamide;
(S)—N-[1-Benzyl-2-(3,4-dihydro-1H-isoquinolin-2-yl)-2-oxo-ethyl]-3-(2-methoxy-pyrimidin-5-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(2,5-dimethyl-2H-pyrazol-3-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(2,5-dimethyl-oxazol-4-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(2,3-dimethyl-3H-imidazol-4-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(5-methyl-pyridin-2-yl)-N-(4-pyrimidin-5-yl-benzyl)-acrylamide;
(S)—N-[1-Benzyl-2-(3,4-dihydro-1H-isoquinolin-2-yl)-2-oxo-ethyl]-3-(6-chloro-pyridin-3-yl)-N-(4-pyrimidin-5-yl-benzyl)-acrylamide;
(S)—N-[1-Benzyl-2-(3,4-dihydro-1H-isoquinolin-2-yl)-2-oxo-ethyl]-3-(6-methoxy-pyridin-3-yl)-N-(4-pyrimidin-5-yl-benzyl)-acrylamide;
(S)—N-[1-Benzyl-2-(3,4-dihydro-1H-isoquinolin-2-yl)-2-oxo-ethyl]-N-(4-pyrimidin-5-yl-benzyl)-3-(5-trifluoromethyl-pyridin-2-yl)-acrylamide;
(S)—N-[1-Benzyl-2-(3,4-dihydro-1H-isoquinolin-2-yl)-2-oxo-ethyl]-3-(2-methyl-pyrimidin-5-yl)-N-(4-pyrimidin-5-yl-benzyl)-acrylamide;
(S)—N-[1-Benzyl-2-(3,4-dihydro-1H-isoquinolin-2-yl)-2-oxo-ethyl]-3-(2,4-dimethyl-thiazol-5-yl)-N-(4-pyrimidin-5-yl-benzyl)-acrylamide;
(S)—N-[1-Benzyl-2-(3,4-dihydro-1H-isoquinolin-2-yl)-2-oxo-ethyl]-3-(2-methyl-thiazol-4-yl)-N-(4-pyrimidin-5-yl-benzyl)-acrylamide;
(S)—N-[1-Benzyl-2-(3,4-dihydro-1H-isoquinolin-2-yl)-2-oxo-ethyl]-3-(5-methyl-isoxazol-3-yl)-N-(4-pyrimidin-5-yl-benzyl)-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-(4-pyrimidin-5-yl-benzyl)-acrylamide;
(S)—N-[1-Benzyl-2-(3,4-dihydro-1H-isoquinolin-2-yl)-2-oxo-ethyl]-3-(5-methoxy-pyridin-2-yl)-N-(4-pyridin-4-yl-benzyl)-acrylamide;
(S)—N-[1-Benzyl-2-(3,4-dihydro-1H-isoquinolin-2-yl)-2-oxo-ethyl]-3-(5-methyl-pyridin-2-yl)-N-(4-pyridin-4-yl-benzyl)-acrylamide;
(S)—N-[1-Benzyl-2-(3,4-dihydro-1H-isoquinolin-2-yl)-2-oxo-ethyl]-3-(2-methyl-pyrimidin-5-yl)-N-(4-pyridin-4-yl-benzyl)-acrylamide;
(S)—N-[1-Benzyl-2-(3,4-dihydro-1H-isoquinolin-2-yl)-2-oxo-ethyl]-3-(6-methoxy-pyridazin-3-yl)-N-(4-pyridin-4-yl-benzyl)-acrylamide;
(S)—N-[1-Benzyl-2-(3,4-dihydro-1H-isoquinolin-2-yl)-2-oxo-ethyl]-N-(4-pyridin-4-yl-benzyl)-3-[1,2,3]thiadiazol-4-yl-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-(4-pyridin-4-yl-benzyl)-acrylamide;
(S)—N-[1-Benzyl-2-(3,4-dihydro-1H-isoquinolin-2-yl)-2-oxo-ethyl]-3-(2,5-dimethyl-oxazol-4-yl)-N-(4-pyridin-4-yl-benzyl)-acrylamide;
(S)—N-[1-Benzyl-2-(3,4-dihydro-1H-isoquinolin-2-yl)-2-oxo-ethyl]-3-(1-methyl-1H-pyrazol-3-yl)-N-(4-pyridin-4-yl-benzyl)-acrylamide;
(S)—N-[1-Benzyl-2-(3,4-dihydro-1H-isoquinolin-2-yl)-2-oxo-ethyl]-3-(2,5-dimethyl-2H-pyrazol-3-yl)-N-(4-pyridin-4-yl-benzyl)-acrylamide;
(S)—N-[1-Benzyl-2-(3,4-dihydro-1H-isoquinolin-2-yl)-2-oxo-ethyl]-3-(1,3,5-trimethyl-1H-pyrazol-4-yl)-N-(4-pyridin-4-yl-benzyl)acrylamide;
(S)—N-[1-Benzyl-2-(3,4-dihydro-1H-isoquinolin-2-yl)-2-oxo-ethyl]-3-(2,3-dimethyl-3H-imidazol-4-yl)-N-(4-pyridin-4-yl-benzyl)-acrylamide;
(S)—N-[1-Benzyl-2-(3,4-dihydro-1H-isoquinolin-2-yl)-2-oxo-ethyl]-3-(2,4-dimethyl-thiazol-5-yl)-N-(4-pyridin-4-yl-benzyl)-acrylamide;
(S)—N-[1-Benzyl-2-(3,4-dihydro-1H-isoquinolin-2-yl)-2-oxo-ethyl]-3-(2-methyl-thiazol-4-yl)-N-(4-pyridin-4-yl-benzyl)-acrylamide;
(S)—N-[1-Benzyl-2-(3,4-dihydro-1H-isoquinolin-2-yl)-2-oxo-ethyl]-3-(1,5-dimethyl-1H-pyrazol-4-yl)-N-(4-pyridin-4-yl-benzyl)-acrylamide;
(S)—N-[1-Benzyl-2-(3,4-dihydro-1H-isoquinolin-2-yl)-2-oxo-ethyl]-N-(4-pyridin-4-yl-benzyl)-3-(5-trifluoromethyl-pyridin-2-yl)acrylamide;
(S)—N-[1-Benzyl-2-(3,4-dihydro-1H-isoquinolin-2-yl)-2-oxo-ethyl]-3-(2,4-dimethyl-thiazol-5-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(2-methyl-thiazol-4-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(2,5-dimethyl-oxazol-4-yl)-N-(4-pyrimidin-5-yl-benzyl)-acrylamide;
(S)—N-[1-Benzyl-2-(3,4-dihydro-1-H-isoquinolin-2-yl)-2-oxo-ethyl]-N-[4-(1-oxy-pyridin-4-yl)-benzyl]-3-(4-trifluoromethyl-phenyl)-acrylamide;
N-[2-(3,4-Dihydro-1H-isoquinolin-2-yl)-1-(1-methyl-1H-pyrazol-4-ylmethyl)-2-oxo-ethyl]-N-(4-pyridin-2-yl-benzyl)-3-(4-trifluoromethyl-phenyl)-acrylamide, such as especially (S)—N-[2-(3,4-Dihydro-1H-isoquinolin-2-yl)-1-(1-methyl-1H-pyrazol-4-ylmethyl)-2-oxo-ethyl]-N-(4-pyridin-2-yl-benzyl)-3-(4-trifluoromethyl-phenyl)-acrylamide;
N-[2-(3,4-Dihydro-1H-isoquinolin-2-yl)-1-(1-methyl-1H-pyrazol-4-ylmethyl)-2-oxo-ethyl]-N-(4-pyridin-4-yl-benzyl)-3-(4-trifluoromethyl-phenyl)-acrylamide, such as especially (S)—N-[2-(3,4-Dihydro-1H-isoquinolin-2-yl)-1-(1-methyl-1H-pyrazol-4-ylmethyl)-2-oxo-ethyl]-N-(4-pyridin-4-yl-benzyl)-3-(4-trifluoromethyl-phenyl)-acrylamide;
N-[2-(3,4-Dihydro-1H-isoquinolin-2-yl)-1-(1-methyl-1H-pyrazol-3-ylmethyl)-2-oxo-ethyl]-N-(4-pyridin-2-yl-benzyl)-3-(4-trifluoromethyl-phenyl)-acrylamide, such as especially (S)—N-[2-(3,4-Dihydro-1H-isoquinolin-2-yl)-1-(1-methyl-1H-pyrazol-3-ylmethyl)-2-oxo-ethyl]-N-(4-pyridin-2-yl-benzyl)-3-(4-trifluoromethyl-phenyl)-acrylamide;
N-[2-(3,4-Dihydro-1H-isoquinolin-2-yl)-1-(1-methyl-1H-pyrazol-3-ylmethyl)-2-oxo-ethyl]-N-(4-pyridin-4-yl-benzyl)-3-(4-trifluoromethyl-phenyl)-acrylamide, such as especially (S)—N-[2-(3,4-Dihydro-1H-isoquinolin-2-yl)-1-(1-methyl-1H-pyrazol-3-ylmethyl)-2-oxo-ethyl]-N-(4-pyridin-4-yl-benzyl)-3-(4-trifluoromethyl-phenyl)-acrylamide;
N-[2-(3,4-Dihydro-1H-isoquinolin-2-yl)-1-(2-methyl-2H-pyrazol-3-ylmethyl)-2-oxo-ethyl]-N-(4-pyridin-2-yl-benzyl)-3-(4-trifluoromethyl-phenyl)-acrylamide, such as especially (S)—N-[2-(3,4-Dihydro-1H-isoquinolin-2-yl)-1-(2-methyl-2H-pyrazol-3-ylmethyl)-2-oxo-ethyl]-N-(4-pyridin-2-yl-benzyl)-3-(4-trifluoromethyl-phenyl)-acrylamide;
N-[2-(3,4-Dihydro-1H-isoquinolin-2-yl)-1-(2-methyl-2H-pyrazol-3-ylmethyl)-2-oxo-ethyl]-N-(4-pyridin-4-yl-benzyl)-3-(4-trifluoromethyl-phenyl)-acrylamide, such as especially (S)—N-[2-(3,4-Dihydro-1H-isoquinolin-2-yl)-1-(2-methyl-2H-pyrazol-3-ylmethyl)-2-oxo-ethyl]-N-(4-pyridin-4-yl-benzyl)-3-(4-trifluoromethyl-phenyl)-acrylamide;
N-[2-(3,4-Dihydro-1H-isoquinolin-2-yl)-2-oxo-1-pyrimidin-2-ylmethyl-ethyl]-N-(4-pyridin-2-yl-benzyl)-3-(4-trifluoromethyl-phenyl)-acrylamide, such as especially (S)—N-[2-(3,4-Dihydro-1H-isoquinolin-2-yl)-2-oxo-1-pyrimidin-2-ylmethyl-ethyl]-N-(4-pyridin-2-yl-benzyl)-3-(4-trifluoromethyl-phenyl)-acrylamide;
N-[2-(3,4-Dihydro-1H-isoquinolin-2-yl)-2-oxo-1-pyrimidin-2-ylmethyl-ethyl]-N-(4-pyridin-4-yl-benzyl)-3-(4-trifluoromethyl-phenyl)-acrylamide, such as especially (S)—N-[2-(3,4-Dihydro-1H-isoquinolin-2-yl)-2-oxo-1-pyrimidin-2-ylmethyl-ethyl]-N-(4-pyridin-4-yl-benzyl)-3-(4-trifluoromethyl-phenyl)-acrylamide; and
N-[2-(3,4-Dihydro-1H-isoquinolin-2-yl)-1-isoxazol-3-ylmethyl-2-oxo-ethyl]-N-(4-pyridin-4-yl-benzyl)-3-(4-trifluoromethyl-phenyl)-acrylamide, such as especially (S)—N-[2-(3,4-Dihydro-1H-isoquinolin-2-yl)-1-isoxazol-3-ylmethyl-2-oxo-ethyl]-N-(4-pyridin-4-yl-benzyl)-3-(4-trifluoromethyl-phenyl)-acrylamide.

The compounds of formula I and their pharmaceutically acceptable salts can be used as medicaments, e.g. in the form of pharmaceutical compositions for enteral or parenteral administration, and are suitable for the treatment and/or prevention of the diseases mentioned herein, such as especially malaria.

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the described compounds of formula I or their pharmaceutically acceptable salts, optionally in combination with other therapeutically valuable substances, into a glacial administration form together with suitable, non-toxic, inert, pharmaceutically acceptable solid or liquid carrier materials and, if desired, usual pharmaceutical adjuvants.

In one embodiment, the invention relates to a method for the treatment or prevention of the diseases mentioned herein, such as especially malaria, said method comprising administering to a subject a pharmaceutically active amount of a compound of formula I.

The compounds of formula I or the above-mentioned pharmaceutical compositions may also be used in combination with one or more other therapeutically useful substances e.g. with other antimalarials like quinolines (e.g. quinine, chloroquine, amodiaquine, mefloquine, primaquine, and tafenoquine), peroxide antimalarials (e.g. artemisinin, artemether, and artesunate), pyrimethamine-sulfadoxine antimalarials (e.g. Fansidar®), hydroxynaphtoquinones (e.g. atovaquone), acroline-type antimalarials (e.g. pyronaridine), and other antiprotozoal agents like ethylstibamine, hydroxystilbamidine, pentamidine, stilbamidine, quinapyramine, puromycin, propamidine, nifurtimox, melarsoprol, nimorazole, nifuroxime, aminitrozole and the like.

The present invention also relates to the use of a compound of formula I for the preparation of a pharmaceutical composition, optionally for use in combination with one or more other therapeutically useful substances such as those mentioned in the preceding paragraph, for the prevention and/or treatment of the diseases mentioned herein, such as especially malaria.

The compounds of the formula I of the present invention may be prepared according to the procedures described herein, especially as described in the experimental part.

In general, all chemical transformations can be performed according to well-known standard methodologies as described in the literature or as described in the procedures below.

### Preparation of Compounds of Formula I:

### Method A:

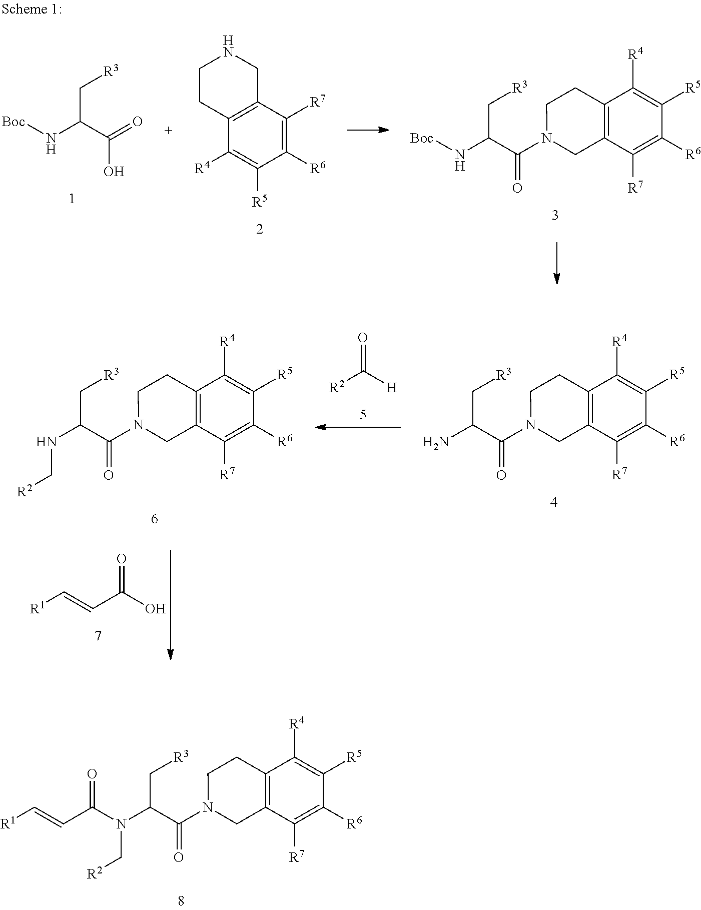

The Boc-protected aminoacid 1 can be coupled with a tetrahydroisoquinoline derivative 2 by the help of a coupling/activating reagent such as TBTU in a solvent such as DCM, at rt in the presence of a base such as DIPEA, to give the intermediate 3. Boc-deprotection is usually achieved by reacting 3 with TFA in DCM to give the amine intermediate 4. Reductive amination with an aldehyde derivative 5 in a solvent such as DCM and in the presence of a reducing reagent such as sodium triacetoxyborohydride gives the expected secondary amine intermediate 6. Compound 6 can be acylated by a carboxylic acid 7 by the help of a coupling/activating reagent such as TBTU in a solvent such as DCM, at rt in the presence of a base such as DIPEA, to give the final compounds 8 of formula I.

The compounds of formula I can also be prepared via method B and according to Scheme 2.

Method B:

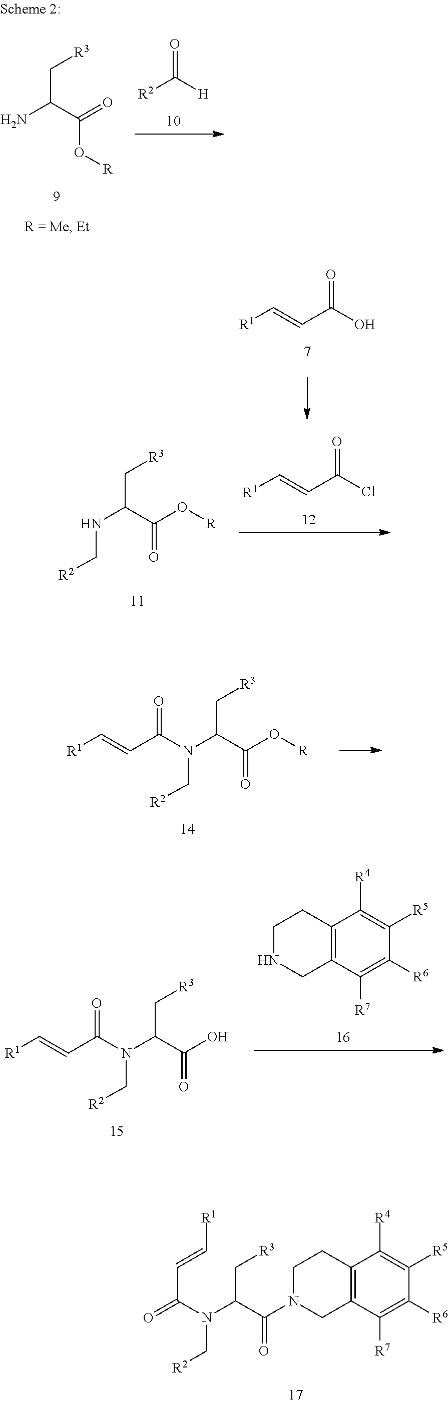

Reductive amination of an amino-acid 9 with an aldehyde derivative 10 in a solvent such as MeOH and in the presence of a reducing reagent such as sodium borohydride gives the expected secondary amine intermediate 11. Compound 11 can be acylated by an acyl chloride 12 in a solvent such as DCM in the presence of a base such as DIPEA, to give the amide intermediate 14.

The acyl chloride can be generated by reaction of the corresponding carboxylic acid 7 with oxalyl chloride in the presence of few drops of DMF and in a solvent such as DCM. Saponification of the ester function using methods known in the art such as treatment with a base such as NaOH in solvent mixtures such as methanol/water or ethanol/water followed by acylation of the resulting acid 15 with a 1,2,3,4-tetrahydroisoquinoline derivative 16 with the help of a coupling/activating reagent such as TBTU in a solvent such as DCM in the presence of a base such as DIPEA provides the final compounds 17 of formula I.

Carboxylic acid compounds 7 are commercially available or can be synthetised according to the following pathways:

### Pathway A: Knoevenagel Reaction

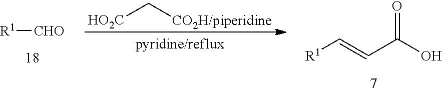

### Pathway B: Horner-Emmons Reaction

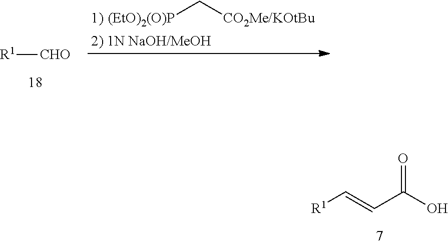

### Pathway C: Heck Reaction

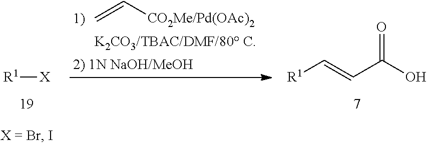

Pathway A: By reaction of an aldehyde 18 with malonic acid in the presence of a strong base such as piperidine in refluxing pyridine furnishes the desired carboxylic acid 7.

Pathway B: By reaction of an aldehyde 18 with trimethyl phosphoacetate in the presence of a strong base such as KOtBu in an aprotic solvent such as THF followed by saponification of the resulting methyl ester with 1N NaOH in MeOH furnishes the desired carboxylic acid 7.

Pathway C: By reaction of a halide 19 with methyl acrylate in the presence of a base such as potassium carbonate, a palladium catalyst such as palladium (II) acetate and a phase-transfer catalyst TBAC in DMF followed by saponification of the resulting methyl ester with 1N NaOH in MeOH provides the desired carboxylic acid 7.

Amino acid derivatives 9 are commercially available or can be synthetised according to the following pathways:

Pathway D: via Horner-Wadsworth-Emmons reaction

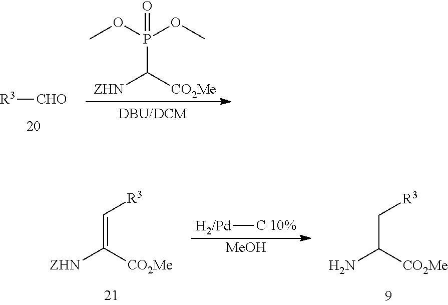

Pathway E: via alkylation of glycinate ester shift base

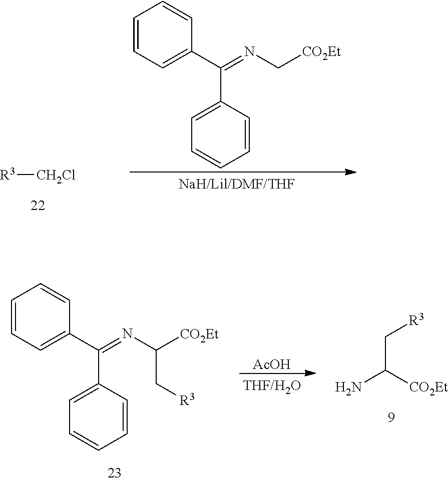

Pathway D: Reaction of aldehyde derivative 20 with commercially available (+/−)-Z-α-phosphonoglycine-trimethylester in the presence of a strong base such as DBU in an aprotic solvent such as DCM affords the intermediate 21 (WO 2007/070826). Hydrogenation over Pd—C 10% in MeOH gives the desired amino-acid derivatives 9 (US 2007/0149503).

Pathway E: Alkylation of commercially available N-(diphenylmethylene)-glycine ethyl ester with a chloride derivative R³—CH₂Cl 22 in the presence of a strong base such as NaH and LiI in a mixture of DMF/THF affords the intermediate 23 (WO 2005/016883 and WO 2006/045613). Acidic hydrolysis gives the desired amino-acid derivatives 9 (WO 2005/016883 and WO 2001/68591).

Aldehyde derivatives 10 are commercially available or can be synthetised according to the following pathways:

Pathway F: via palladium cross-coupling reaction

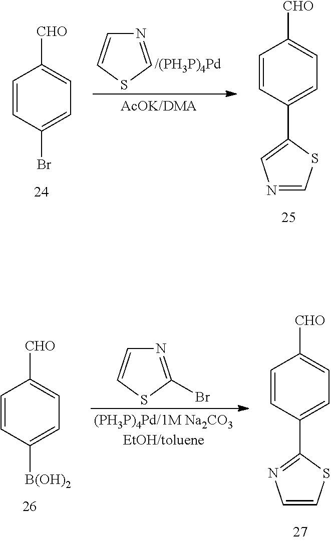

Pathway G: via cyclisation

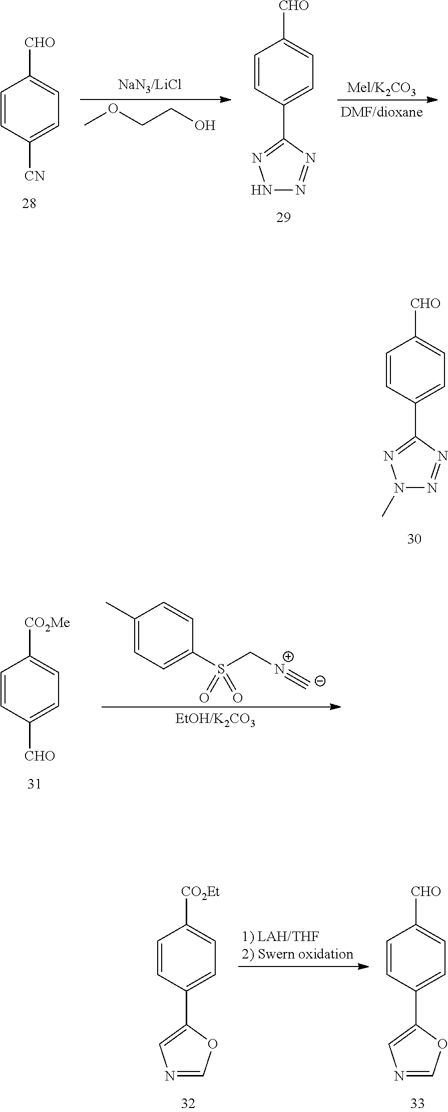

Pathway F: Reaction of commercially available 4-bromobenzaldehyde 24 with commercially available thiazole in the presence of a palladium catalyst such as tetrakis(triphenylphosphine)palladium, and a base such as potassium acetate in an aprotic solvent such as DMA affords the desired 4-thiazol-5-yl-benzaldehyde 25 (Bold G. et al J. Med. Chem. 1998, 41, 18, 3387-3401).

Suzuki reaction of commercially available 4-formylbenzeneboronic acid 26 with commercially available 2-bromo-thiazole in the presence of a palladium catalyst such as tetrakis(triphenylphosphine)palladium, and a base such as 1M Na₂CO₃ in a mixture of EtOH/toluene gives the desired 4-thiazol-2-yl-benzaldehyde 27 (Bold G. et al J. Med. Chem. 1998, 41, 18, 3387-3401).

Pathway G: Reaction of commercially available 4-cyanobenzaldehyde 28 with NaN₃ in the presence of LiCl in methoxyethanol affords the desired 4-(2H-tetrazol-5-yl)-benzaldehyde 29. Methylation with MeI in the presence of a base such as K₂CO₃ in a DMF/dioxane mixture gives the desired 4-(2-methyl-2H-tetrazol-5-yl)-benzaldehyde 30 (Bold G. et al J. Med. Chem. 1998, 41, 18, 3387-3401).

Reaction of commercially available methyl-4-formylbenzoate 31 with toluenesulfonylmethyl isocyanide in the presence of a base such as K₂CO₃ in EtOH affords 4-oxazol-5-yl-benzoic acid ethyl ester 32. Reduction with LAH in an aprotic solvent such as THF, followed by Swern oxidation ((COCl)₂/TEA/DMSO/DCM) gives the desired 4-oxazol-5-yl-benzaldehyde 33 (Tanaka A. et al J. Med. Chem. 1998, 41, 2190-2410).

The following examples illustrate the invention but do not limit the scope thereof. All temperatures are stated in ° C.

Abbreviations (as used herein):

AcOH acetic acid
AcOK potassium acetate
aq. aqueous
Boc tert.-butyloxycarbonyl
DBU 1,8-diazabicyclo[5,4,0]undec-7-ene
DCM dichloromethane

### DMA N,N-dimethylacetamide

DIPEA diisopropylethylamine
DMF dimethylformamide
DMSO dimethylsulfoxide
EA ethyl acetate
ELSD evaporative light scattering detection
Et ethyl
EtOH ethanol
FC flash chromatography
h hour(s)
HPLC high performance liquid chromatography
KOtBu potassium tert-butoxide
LAH lithium aluminium hydride
LC-MS liquid chromatography—mass spectroscopy
m-CPBA 3-chloroperoxybenzoic acid
Me methyl
MeOH methanol
min minute(s)
MS mass spectroscopy
N normality
OAc acetate
PBS phosphate buffered saline
Ph phenyl
rt room temperature
sat. saturated
TBAC tetra-n-butylammonium chloride
TBTU O-benzotriazol-1-yl-N,N,N′,N′-tetramethyluronium tetrafluoroborate
TEA triethylamine
TFA trifluoroacetic acid
THF tetrahydrofuran
t_{R} retention time
UV ultra violet
V is visible
Z benzyloxycarbonyl

### General Procedures and Examples:

HPLC conditions:

Analytic: Zorbax 59 SB Aqua column, 4.6×50 mm from Agilent Technologies. Eluents: A: acetonitrile; B: H₂O+0.5% TFA. Gradient: 90% B→5% B over 2 min. Flow: 1 mL/min. Detection: UV/Vis+MS.

Preparative: Zorbax SB Aqua column, 20×500 mm from Agilent Technologies. Eluent: A: Acetonitrile; B: H₂O+0.05% ammonium hydroxide (25% aq.). Gradient: 80% B→10% B over 6 min. Flow: 40 mL/min. Detection: UV+MS, or UV+ELSD.

Chiral, analytic: Regis Whelk column, 4.6×250 mm, 10 μm. Eluent A: EtOH+0.05% Et₃N. Eluent B: hexane. Isocratic conditions, usually 60% B, over 40 min, 1 mL/min. The isocratic mixture may vary, depending on the compounds.

Chiral, preparative: As analytical conditions, but on a Regis Whelk 01 column, 50×250 mm and a flow of 100 mL/min.

### Preparation of Compound of Formula I Via Method A

### Example 1

### Step 1: (S)-[1-Benzyl-2-(3,4-dihydro-1H-isoquinolin-2-yl)-2-oxo-ethyl]-carbamic acid tert-butyl ester

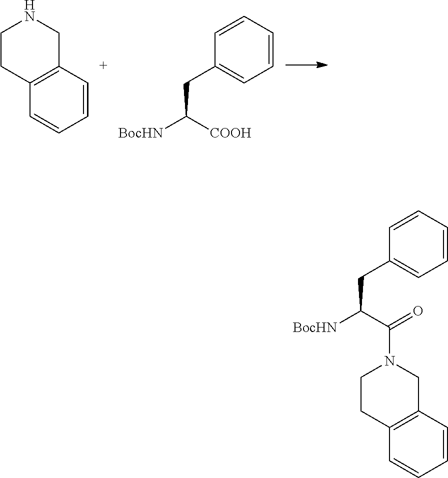

To a solution of Boc-L-phenylalanine (4.01 g), and DIPEA (13.1 mL) in dry DCM (18 mL) was added TBTU (4.81 g). The reaction mixture was stirred at rt for 30 min and then a solution of 1,2,3,4-tetrahydroisoquinoline (2.1 g) in dry DCM (10 mL) was added. The reaction mixture was stirred at rt for 16 h and concentrated in vacuo. The resulting residue was dissolved in EA, washed with 1N HCl, water, sat. NaHCO₃, and brine, dried (MgSO₄), filtered and concentrated to yield a crude oil.

FC (EA/n-Heptane: 1/9 to 2/8) gave the title compound (4.01 g, 70%) as a white foam. LC-MS: t_{R}=1.03 min; [M+H]⁺=381.15.

### Step 2: (S)-2-amino-1-(3,4-dihydro-1H-isoquinolin-2-yl)-3-phenyl-propan-1-one

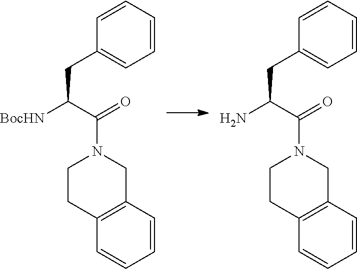

To a cold (0° C.) solution of (S)-[1-benzyl-2-(3,4-dihydro-1H-isoquinolin-2-yl)-2-oxo-ethyl]-carbamic acid tert-butyl ester (2.66 g) in dry DCM (90 mL) was added dropwise TFA (5.35 mL). The reaction was stirred at rt for 16 h and then concentrated in vacuo. The resulting residue was dissolved in DCM, washed with sat. NaHCO₃, and the aq. phase was extracted twice with DCM. The combined organic extracts were washed with brine, dried (MgSO₄), filtered and concentrated to yield the title compound as an oil (1.95 g, 99%). LC-MS: t_{R}=0.74 min; [M+H]⁺=281.09.

### Step 3: (S)-1-(3,4-Dihydro-1H-isoquinolin-2-yl)-3-phenyl-2-(4-pyridin-2-yl-benzylamino)-propan-1-one

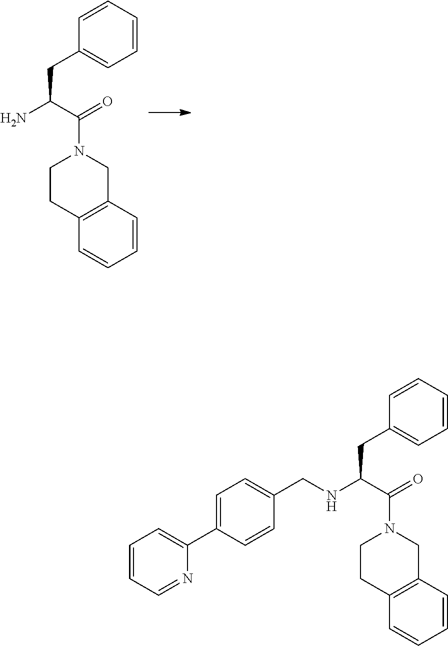

To a solution of (S)-2-amino-1-(3,4-dihydro-1H-isoquinolin-2-yl)-3-phenyl-propan-1-one (0.72 g) in dry DCM (30 mL) were added successively 4-(2-pyridyl)-benzaldehyde (0.31 g) and sodium triacetoxyborohydride (1.16 g). The reaction mixture was stirred at rt for 16 h and washed with sat. NH₄Cl. The organic phase was washed with brine, dried (MgSO₄), filtered and concentrated to yield a crude oil. FC (EA/n-heptane: 25/75 to EA/MeOH: 98/2) gave the title compound as a white solid (0.475 g, 65%).

LC-MS: t_{R}=0.73 min; [M+H]⁺=448.24.

### Step 4: (S)—N-[1-Benzyl-2-(3,4-dihydro-1H-isoquinolin-2-yl)-2-oxo-ethyl]-N-(4-pyridin-2-yl-benzyl)-3-(trifluoromethyl-phenyl)-acrylamide

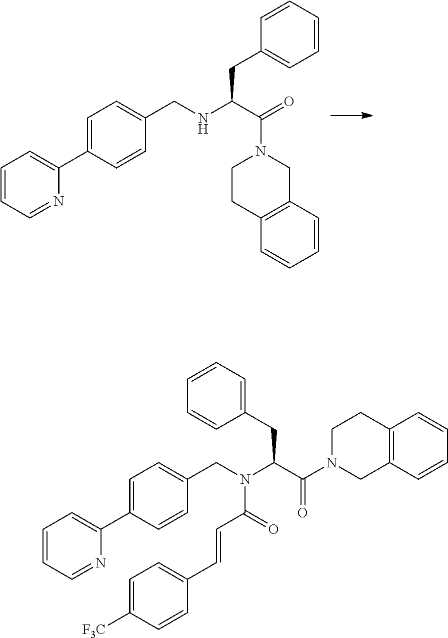

To a solution of trans-4-trifluoromethylcinnamic acid (0.056 mmol), and DIPEA (0.280 mmol) in dry DMF (0.5 mL) was added TBTU (0.056 mmol). The reaction mixture was stirred at rt for 30 min and then a solution of (S)-1-(3,4-dihydro-1H-isoquinolin-2-yl)-3-phenyl-2-(4-pyridin-2-yl-benzylamino)-propan-1-one (0.056 mmol) in dry DMF (0.5 mL) was added. The reaction mixture was stirred at rt for 16 h and the product was purified by preparative HPLC to give the title compound (Example 1) as a beige solid.

LC-MS: t_{R}=1.00 min; [M+H]⁺=646.28.

According to the procedures described above the following examples could be prepared:

### Example 2

### (S)—N-[1-Benzyl-2-(3,4-dihydro-1H-isoquinolin-2-yl)-2-oxo-ethyl]-N-(4-pyridin-2-yl-benzyl)-3-(6-trifluoromethyl-pyridin-3-yl)-acrylamide

This compound has been prepared according to the methods of example 1 but using 3-(6-trifluoromethyl-pyridin-3-yl)-acrylic acid for the step 4.

LC-MS: t_{R}=0.99 min; [M+H]⁺=647.23.

### Example 3

### (S)—N-[1-Benzyl-2-(3,4-dihydro-1H-isoquinolin-2-yl)-2-oxo-ethyl]-N-(4-pyrimidin-5-yl-benzyl)-3-(4-trifluoromethyl-phenyl)-acrylamide

This compound has been prepared according to the methods of example 1 but using 5-(4-formylphenyl)pyrimidine for the step 3.

LC-MS: t_{R}=1.04 min; [M+H]⁺=682.49.

### Example 4

### (S)—N-[1-Benzyl-2-(3,4-dihydro-1H-isoquinolin-2-yl)-2-oxo-ethyl]-N-(4-pyrimidin-5-yl-benzyl)-3-(6-trifluoromethyl-pyridin-3-yl)-acrylamide

This compound has been prepared according to the methods of example 1 but using 5-(4-formylphenyl)pyrimidine for the step 3 and 3-(6-trifluoromethyl-pyridin-3-yl)acrylic acid for the step 4.

LC-MS: t_{R}=1.20 min; [M+H]⁺=648.29.

### Example 5

### (S)—N-[1-Benzyl-2-(3,4-dihydro-1H-isoquinolin-2-yl)-2-oxo-ethyl]-N-(6-morpholin-4-yl-pyridin-3-ylmethyl)-3-(4-trifluoromethyl-phenyl)-acrylamide

This compound has been prepared according to the methods of example 1 but using 6-morpholinonicotinaldehyde for the step 3.

LC-MS: t_{R}=0.98 min; [M+H]⁺=655.14.

### Example 6

### (S)—N-[1-Benzyl-2-(3,4-dihydro-1H-isoquinolin-2-yl)-2-oxo-ethyl]-N-pyridin-2-ylmethyl-3-(4-trifluoromethyl-phenyl)-acrylamide

This compound has been prepared according to the methods of example 1 but using 2-pyridinecarbaldehyde for the step 3.

LC-MS: t_{R}=1.00 min; [M+H]⁺=570.23.

### Example 7

### (S)—N-Benzyl-N-[1-benzyl-2-(3,4-dihydro-1H-isoquinolin-2-yl)-2-oxo-ethyl]-3-(4-trifluoromethyl-phenyl)-acrylamide

This compound has been prepared according to the methods of example 1 but using benzaldehyde for the step 3.

LC-MS: t_{R}=1.15 min; [M+H]⁺=569.23.

### Example 8

### (S)—N-[1-Benzyl-2-(3,4-dihydro-1H-isoquinolin-2-yl)-2-oxo-ethyl]-3-(4-methoxy-phenyl)-N-(6-morpholin-4-yl-pyridin-3-ylmethyl)-acrylamide

This compound has been prepared according to the methods of example 1 but using 6-morpholinonicotinaldehyde for the step 3 and 4-methoxycinnamic acid for the step 4.

LC-MS: t_{R}=0.94 min; [M+H]⁺=617.11.

### Example 9

### (S)—N-[1-Benzyl-2-(6,7-ethoxy-3,4-dihydro-1H-isoquinolin-2-yl)-2-oxo-ethyl]-N-(4-pyridin-2-yl-benzyl)-3-(4-trifluoromethyl-phenyl)-acrylamide

This compound has been prepared according to the methods of example 1 but using 6,7-diethoxy-1,2,3,4-tetrahydroisoquinoline for the step 1.

LC-MS: t_{R}=1.02 min; [M+H]⁺=734.34.

### Example 10

### (S)—N-[1-Benzyl-2-(6-fluoro-3,4-dihydro-1H-isoquinolin-2-yl)-2-oxo-ethyl]-N-(4-pyridin-2-yl-benzyl)-3-(4-trifluoromethyl-phenyl)-acrylamide

This compound has been prepared according to the methods of example 1 but using 6-fluoro-1,2,3,4-tetrahydroisoquinoline for the step 1.

LC-MS: t_{R}=1.01 min; [M+H]⁺=664.27.

### Example 11

### (S)—N-[1-Benzyl-2-(3,4-dihydro-1-H-isoquinolin-2-yl)-2-oxo-ethyl]-3-(3-methoxy-phenyl)-N-(4-pyridin-2-yl-benzyl)-acrylamide

This compound has been prepared according to the methods of example 1 but using 3-(3-methoxy-phenyl)-acrylic acid for the step 4.

LC-MS: t_{R}=0.94 min; [M+H]⁺=608.34.

### Example 12

### (S)—N-[1-Benzyl-2-(3,4-dihydro-1-H-isoquinolin-2-yl)-2-oxo-ethyl]-3-(4-methoxy-phenyl)-N-(4-pyridin-2-yl-benzyl)-acrylamide

This compound has been prepared according to the methods of example 1 but using 3-(4-methoxy-phenyl)-acrylic acid for the step 4.

LC-MS: t_{R}=0.94 min; [M+H]⁺=608.34.

### Example 13

### (S)-3-Benzo[1,3]dioxol-5-yl-N-[benzyl-2-(3,4-dihydro-1H-isoquinolin-2-yl)-2-oxo-ethyl]-N-(4-pyridin-2-yl-benzyl)-acrylamide

This compound has been prepared according to the methods of example 1 but using 3-benzo[1,3]dioxol-5-yl-acrylic acid for the step 4.

LC-MS: t_{R}=0.94 min; [M+H]⁺=622.32.

### Example 14

### (S)—N-[1-Benzyl-2-(3,4-dihydro-1-H-isoquinolin-2-yl)-2-oxo-ethyl]-3-(3,5-dimethoxy-phenyl)-N-(4-pyridin-2-yl-benzyl)-acrylamide

This compound has been prepared according to the methods of example 1 but using 3-(3,5-dimethoxy-phenyl)-acrylic acid for the step 4.

LC-MS: t_{R}=0.99 min; [M+H]⁺=638.38.

### Example 15

### (S)—N-[1-Benzyl-2-(3,4-dihydro-1-H-isoquinolin-2-yl)-2-oxo-ethyl]-3-(4-fluoro-phenyl)-N-(4-pyridin-2-yl-benzyl)-acrylamide

This compound has been prepared according to the methods of example 1 but using 3-(4-fluoro-phenyl)-acrylic acid for the step 4.

LC-MS: t_{R}=0.93 min; [M+H]⁺=596.69.

### Example 16

### (S)—N-[1-Benzyl-2-(3,4-dihydro-1-H-isoquinolin-2-yl)-2-oxo-ethyl]-3-(2,4-difluoro-phenyl)-N-(4-pyridin-2-yl-benzyl)-acrylamide

This compound has been prepared according to the methods of example 1 but using 3-(2,4-difluoro-phenyl)-acrylic acid for the step 4.

LC-MS: t_{R}=0.97 min; [M+H]⁺=614.32.

### Example 17

### (S)—N-[1-Benzyl-2-(3,4-dihydro-1-H-isoquinolin-2-yl)-2-oxo-ethyl]-3-(2-bromo-phenyl)-N-(4-pyridin-2-yl-benzyl)-acrylamide

This compound has been prepared according to the methods of example 1 but using 3-(2-bromo-phenyl)-acrylic acid for the step 4.

LC-MS: t_{R}=0.98 min; [M+H]⁺=656.26.

### Example 18

### (S)—N-[1-Benzyl-2-(3,4-dihydro-1-H-isoquinolin-2-yl)-2-oxo-ethyl]-N-(4-pyridin-2-yl-benzyl)-3-(3-trifluoromethyl-phenyl)-acrylamide

This compound has been prepared according to the methods of example 1 but using 3-(3-trifluoromethyl-phenyl)-acrylic acid for the step 4.

LC-MS: t_{R}=0.99 min; [M+H]⁺=646.33.

### Example 19

### (S)—N-[1-Benzyl-2-(3,4-dihydro-1-H-isoquinolin-2-yl)-2-oxo-ethyl]-3-(3,4-difluoro-phenyl)-N-(4-pyridin-2-yl-benzyl)-acrylamide

This compound has been prepared according to the methods of example 1 but using 3-(3,4-difluoro-phenyl)-acrylic acid for the step 4.

LC-MS: t_{R}=0.97 min; [M+H]⁺=614.30.

### Example 20

### (S)—N-[1-Benzyl-2-(3,4-dihydro-1-H-isoquinolin-2-yl)-2-oxo-ethyl]-3-(3-bromo-phenyl)-N-(4-pyridin-2-yl-benzyl)acrylamide

This compound has been prepared according to the methods of example 1 but using 3-(3-bromo-phenyl)-acrylic acid for the step 4.

LC-MS: t_{R}=0.99 min; [M+H]⁺=656.24.

### Example 21

### (S)—N-[1-Benzyl-2-(3,4-dihydro-1-H-isoquinolin-2-yl)-2-oxo-ethyl]-3-(4-bromo-phenyl)-N-(4-pyridin-2-yl-benzyl)-acrylamide

This compound has been prepared according to the methods of example 1 but using 3-(4-bromo-phenyl)-acrylic acid for the step 4.

LC-MS: t_{R}=0.99 min; [M+H]⁺=656.26.

### Example 22

### (S)—N-[1-Benzyl-2-(3,4-dihydro-1-H-isoquinolin-2-yl)-2-oxo-ethyl]-3-(2,5-dimethyl-phenyl)-N-(4-pyridin-2-yl-benzyl)-acrylamide

This compound has been prepared according to the methods of example 1 but using 3-(2,5-dimethyl-phenyl)-acrylic acid for the step 4.

LC-MS: t_{R}=0.99 min; [M+H]⁺=606.35.

### Example 23

### (S)—N-Benzyl-N-[1-Benzyl-2-(3,4-dihydro-1-H-isoquinolin-2-yl)-2-oxo-ethyl]-3-p-tolyl-acrylamide

This compound has been prepared according to the methods of example 1 but using 3-p-tolyl-acrylic acid for the step 4.

LC-MS: t_{R}=1.18 min; [M+H]⁺=515.65.

### Example 24

### (S)—N-[1-Benzyl-2-(3,4-dihydro-1H-isoquinolin-2-yl)-2-oxo-ethyl]-N-(4-ethyl-benzyl)-3-p-tolyl-acrylamide

This compound has been prepared according to the methods of example 1 but using 3-p-tolyl-acrylic acid for the step 4.

LC-MS: t_{R}=1.21 min; [M+H]⁺=543.71.

### Example 25

### (S)—N-[1-Benzyl-2-(3,4-dihydro-1-H-isoquinolin-2-yl)-2-oxo-ethyl]-3-(2,3-dichloro-phenyl)-N-(4-pyridin-2-yl-benzyl)-acrylamide

This compound has been prepared according to the methods of example 1 but using 3-(2,3-dichloro-phenyl)-acrylic acid for the step 4.

LC-MS: t_{R}=1.11 min; [M+H]⁺=646.19.

### Example 26

### (S)—N-[1-Benzyl-2-(3,4-dihydro-1-H-isoquinolin-2-yl)-2-oxo-ethyl]-3-(2-chloro-phenyl)-N-(4-pyridin-2-yl-benzyl)acrylamide

This compound has been prepared according to the methods of example 1 but using 3-(2-chloro-phenyl)-acrylic acid for the step 4.

LC-MS: t_{R}=1.07 min; [M+H]⁺=612.24.

### Example 27

### (S)—N-[1-Benzyl-2-(3,4-dihydro-1-H-isoquinolin-2-yl)-2-oxo-ethyl]-3-(3-fluoro-phenyl)-N-(4-pyridin-2-yl-benzyl)-acrylamide

This compound has been prepared according to the methods of example 1 but using 3-(3-fluoro-phenyl)-acrylic acid for the step 4.

LC-MS: t_{R}=1.04 min; [M+H]⁺=596.22.

### Example 28

### (S)—N-[1-Benzyl-2-(3,4-dihydro-1-H-isoquinolin-2-yl)-2-oxo-ethyl]-3-(2,5-dimethoxy-phenyl)-N-(4-pyridin-2-yl-benzyl)-acrylamide

This compound has been prepared according to the methods of example 1 but using 3-(2,5-dimethoxy-phenyl)-acrylic acid for the step 4.

LC-MS: t_{R}=1.03 min; [M+H]⁺=638.38.

### Example 29

### (S)—N-[1-Benzyl-2-(3,4-dihydro-1-H-isoquinolin-2-yl)-2-oxo-ethyl]-3-(3,4,5-trimethoxy-phenyl)-N-(4-pyridin-2-yl-benzyl)-acrylamide

This compound has been prepared according to the methods of example 1 but using 3-(3,4,5-trimethoxy-phenyl)-acrylic acid for the step 4.

LC-MS: t_{R}=1.00 min; [M+H]⁺=668.31.

### Example 30

### (S)—N-[1-Benzyl-2-(3,4-dihydro-1-H-isoquinolin-2-yl)-2-oxo-ethyl]-N-(4-pyridin-2-yl-benzyl)-3-o-tolyl-acrylamide

This compound has been prepared according to the methods of example 1 but using 3-o-tolyl-acrylic acid for the step 4.

LC-MS: t_{R}=1.06 min; [M+H]⁺=592.25.

### Example 31

### (S)—N-[1-Benzyl-2-(3,4-dihydro-1-H-isoquinolin-2-yl)-2-oxo-ethyl]-N-(4-pyridin-2-yl-benzyl)-3-m-tolyl-acrylamide

This compound has been prepared according to the methods of example 1 but using 3-m-tolyl-acrylic acid for the step 4.

LC-MS: t_{R}=1.06 min; [M+H]⁺=592.23.

### Example 32

### (S)—N-[1-Benzyl-2-(3,4-dihydro-1-H-isoquinolin-2-yl)-2-oxo-ethyl]-N-(4-pyridin-2-yl-benzyl)-3-p-tolyl-acrylamide

This compound has been prepared according to the methods of example 1 but using 3-p-tolyl-acrylic acid for the step 4.

LC-MS: t_{R}=1.06 min; [M+H]⁺=592.23.

### Example 33

### (S)—N-[1-Benzyl-2-(3,4-dihydro-1-H-isoquinolin-2-yl)-2-oxo-ethyl]-3-(2,4-dichloro-phenyl)-N-(4-pyridin-2-yl-benzyl)-acrylamide

This compound has been prepared according to the methods of example 1 but using 3-(2,4-dichloro-phenyl)-acrylic acid for the step 4.

LC-MS: t_{R}=1.13 min; [M+H]⁺=646.26.

### Example 34

### (S)—N-[1-Benzyl-2-(3,4-dihydro-1-H-isoquinolin-2-yl)-2-oxo-ethyl]-3-(3,4-dichloro-phenyl)-N-(4-pyridin-2-yl-benzyl)-acrylamide

This compound has been prepared according to the methods of example 1 but using 3-(3,4-dichloro-phenyl)-acrylic acid for the step 4.

LC-MS: t_{R}=1.11 min; [M+H]⁺=646.20.

### Example 35

### (S)—N-[1-Benzyl-2-(3,4-dihydro-1-H-isoquinolin-2-yl)-2-oxo-ethyl]-3-(2-methoxy-phenyl)-N-(4-pyridin-2-yl-benzyl)-acrylamide

This compound has been prepared according to the methods of example 1 but using 3-(2-methoxy-phenyl)-acrylic acid for the step 4.

LC-MS: t_{R}=1.04 min; [M+H]⁺=608.23.

### Example 36

### (S)—N-[1-Benzyl-2-(3,4-dihydro-1-H-isoquinolin-2-yl)-2-oxo-ethyl]-3-(4-chloro-phenyl)-N-(4-pyridin-2-yl-benzyl)-acrylamide

This compound has been prepared according to the methods of example 1 but using 3-(4-chloro-phenyl)-acrylic acid for the step 4.

LC-MS: t_{R}=1.07 min; [M+H]⁺=612.22.

### Example 37

### (S)—N-[1-Benzyl-2-(3,4-dihydro-1-H-isoquinolin-2-yl)-2-oxo-ethyl]-3-(3-chloro-phenyl)-N-(4-pyridin-2-yl-benzyl)-acrylamide

This compound has been prepared according to the methods of example 1 but using 3-(3-chloro-phenyl)-acrylic acid for the step 4.

LC-MS: t_{R}=1.07 min; [M+H]⁺=612.20.

### Example 38

### (S)—N-[1-Benzyl-2-(3,4-dihydro-1-H-isoquinolin-2-yl)-2-oxo-ethyl]-3-(4-dimethylamino-phenyl)-N-(4-pyridin-2-yl-benzyl)-acrylamide

This compound has been prepared according to the methods of example 1 but using 3-(4-dimethylamino-phenyl)-acrylic acid for the step 4.

LC-MS: t_{R}=1.05 min; [M+H]⁺=621.30.

### Example 39

### (S)—N-[1-Benzyl-2-(3,4-dihydro-1-H-isoquinolin-2-yl)-2-oxo-ethyl]-N-(4-pyridin-4-yl-benzyl)-3-(4-trifluoromethyl-phenyl)-acrylamide

This compound has been prepared according to the methods of example 1 but using 4-(4-pyridyl)-benzaldehyde for the step 3 and using 4-trifluoromethylcinnamic acid for the step 4.

LC-MS: t_{R}=1.04 min; [M+H]⁺=646.27.

### Example 40

### (S)—N-Benzyl-N-[1-benzyl-2-(3,4-dihydro-1H-isoquinolin-2-yl)-2-oxo-ethyl]-3-(6-trifluoromethyl-pyridin-3-yl)-acrylamide

This compound has been prepared according to the methods of example 7 but using 3-(6-trifluoromethyl-pyridin-3-yl)-acrylic acid for the step 4.

LC-MS: t_{R}=1.11 min; [M+H]⁺=570.26.

### Example 41

### (S)—N-[1-Benzyl-2-(3,4-dihydro-1H-isoquinolin-2-yl)-2-oxo-ethyl]-N-(6-morpholin-4-yl-pyridin-3-ylmethyl)-3-p-tolyl-acrylamide

This compound has been prepared according to the methods of example 1 but using 6-morpholinonicotinaldehyde for the step 3 and 3-p-tolyl-acrylic acid for the step 4.

LC-MS: t_{R}=0.96 min; [M+H]⁺=601.15.

### Example 42

### (S)—N-[1-Benzyl-2-(3,4-dihydro-1H-isoquinolin-2-yl)-2-oxo-ethyl]-N-pyridin-3-ylmethyl-3-p-tolyl-acrylamide

This compound has been prepared according to the methods of example 1 but using (L)-2-tert-butoxycarbonylamino-3-pyridin-2-yl-propionic acid for the step 1,4-ethyl-benzaldehyde for the step 3, and 3-p-tolyl-acrylic acid for the step 4.

LC-MS: t_{R}=0.98 min; [M+H]⁺=544.28.

### Example 43

### (S)—N-[1-Benzyl-2-(3,4-dihydro-1H-isoquinolin-2-yl)-2-oxo-ethyl]-N-pyridin-3-ylmethyl-3-p-tolyl-acrylamide

This compound has been prepared according to the methods of example 1 but using pyridine-3-carbaldehyde for the step 3 and using 3-p-tolyl-acrylic acid for the step 4.

LC-MS: t_{R}=0.93 min; [M+H]⁺=516.68.

### Example 44

### (S)—N-[1-Benzyl-2-(3,4-dihydro-1H-isoquinolin-2-yl)-2-oxo-ethyl]-3-(2,3,6-trifluoro-phenyl)-N-(4-pyridin-2-yl-benzyl)-acrylamide

This compound has been prepared according to the methods of example 1 but using 3-(2,3,6-trifluoro-phenyl)-acrylic acid for the step 4.

LC-MS: t_{R}=1.06 min; [M+H]⁺=632.34.

### Example 45

### (S)—N-[1-Benzyl-2-(3,4-dihydro-1H-isoquinolin-2-yl)-2-oxo-ethyl]-3-(2,5-difluoro-phenyl)-N-(4-pyridin-2-yl-benzyl)-acrylamide

This compound has been prepared according to the methods of example 1 but using 3-(2,3,6-trifluoro-phenyl)-acrylic acid for the step 4.

LC-MS: t_{R}=1.05 min; [M+H]⁺=614.18.

### Example 46

### (S)—N-[1-Benzyl-2-(3,4-dihydro-1H-isoquinolin-2-yl)-2-oxo-ethyl]-3-(2-trifluoromethyl-phenyl)-N-(4-pyridin-2-yl-benzyl)-acrylamide

This compound has been prepared according to the methods of example 1 but using 3-(2-trifluoromethyl-phenyl)-acrylic acid for the step 4.

LC-MS: t_{R}=1.07 min; [M+H]⁺=646.24.

### Example 47

### (S)—N-[1-Benzyl-2-(3,4-dihydro-1H-isoquinolin-2-yl)-2-oxo-ethyl]-3-(1-methyl-1H-pyrazol-3-yl)-N-(4-pyridin-2-yl-benzyl)-acrylamide

This compound has been prepared according to the methods of example 1 but using 3-(1-methyl-1H-pyrazol-3-yl)-acrylic acid for the step 4.

LC-MS: t_{R}=0.86 min; [M+H]⁺=582.11.

### Example 48

### (S)—N-[1-Benzyl-2-(3,4-dihydro-1H-isoquinolin-2-yl)-2-oxo-ethyl]-3-(2,5-dimethyl-2H-pyrazol-3-yl)-N-(4-pyridin-2-yl-benzyl)acrylamide

This compound has been prepared according to the methods of example 1 but using 3-(2,5-dimethyl-2H-pyrazol-3-yl)-acrylic acid for the step 4.

LC-MS: t_{R}=0.90 min; [M+H]⁺=596.20.

### Example 49

### (S)—N-[1-Benzyl-2-(3,4-dihydro-1H-isoquinolin-2-yl)-2-oxo-ethyl]-N-(4-pyridin-2-yl-benzyl)-3-(1,3,5-trimethyl-1H-pyrazol-4-yl)-acrylamide

This compound has been prepared according to the methods of example 1 but using 3-(1,3,5-trimethyl-1H-pyrazol-4-yl)-acrylic acid for the step 4.

LC-MS: t_{R}=0.90 min; [M+H]⁺=610.19.

### Example 50

### (S)—N-[1-Benzyl-2-(3,4-dihydro-1H-isoquinolin-2-yl)-2-oxo-ethyl]-3-(2,3-dimethyl-3H-imidazol-4-yl)-N-(4-pyridin-2-yl-benzyl)-acrylamide

This compound has been prepared according to the methods of example 1 but using 3-(2,3-dimethyl-3H-imidazol-4-yl)-acrylic acid for the step 4.

LC-MS: t_{R}=0.75 min; [M+H]⁺=596.27.

### Example 51

### (S)—N-[1-Benzyl-2-(3,4-dihydro-1H-isoquinolin-2-yl)-2-oxo-ethyl]-3-(2,4-dimethyl-thiazol-5-yl)-N-(4-pyridin-2-yl-benzyl)-acrylamide

This compound has been prepared according to the methods of example 1 but using 3-(2,4-dimethyl-thiazol-5-yl)-acrylic acid for the step 4.

LC-MS: t_{R}=0.94 min; [M+H]⁺=613.15.

### Example 52

### (S)—N-[1-Benzyl-2-(3,4-dihydro-1H-isoquinolin-2-yl)-2-oxo-ethyl]-3-(2-methyl-thiazol-4-yl)-N-(4-pyridin-2-yl-benzyl)-acrylamide

This compound has been prepared according to the methods of example 1 but using 3-(2-methyl-thiazol-4-yl)-acrylic acid for the step 4.

LC-MS: t_{R}=0.89 min; [M+H]⁺=598.90.

### Example 53

### (S)—N-[1-Benzyl-2-(3,4-dihydro-1H-isoquinolin-2-yl)-2-oxo-ethyl]-3-(5-methyl-isoxazol-3-yl)-N-(4-pyridin-2-yl-benzyl)-acrylamide

This compound has been prepared according to the methods of example 1 but using 3-(5-methyl-isoxazol-3-yl)-acrylic acid for the step 4.

LC-MS: t_{R}=0.91 min; [M+H]⁺=582.93.

### Example 54

### (S)—N-[1-Benzyl-2-(3,4-dihydro-1H-isoquinolin-2-yl)-2-oxo-ethyl]-3-(3,5-dimethyl-isoxazol-4-yl)-N-(4-pyridin-2-yl-benzyl)acrylamide

This compound has been prepared according to the methods of example 1 but using 3-(3,5-dimethyl-isoxazol-4-yl)-acrylic acid for the step 4.

LC-MS: t_{R}=0.92 min; [M+H]⁺=597.18.

### Example 55

### (S)—N-[1-Benzyl-2-(3,4-dihydro-1H-isoquinolin-2-yl)-2-oxo-ethyl]-3-(2,5-dimethyl-oxazol-4-yl)-N-(4-pyridin-2-yl-benzyl)-acrylamide

This compound has been prepared according to the methods of example 1 but using 3-(2,5-dimethyl-oxazol-4-yl)-acrylic acid for the step 4.

LC-MS: t_{R}=0.89 min; [M+H]⁺=597.20.

### Example 56

### (S)—N-[1-Benzyl-2-(3,4-dihydro-1H-isoquinolin-2-yl)-2-oxo-ethyl]-N-(4-pyridin-2-yl-benzyl)-3-[1,2,3]thiadiazol-4-yl-acrylamide

This compound has been prepared according to the methods of example 1 but using 3-[1,2,3]-thiadiazol-4-yl-acrylic acid for the step 4.

LC-MS: t_{R}=0.96 min; [M+H]⁺=586.13.

### Example 57

### (S)—N-[1-Benzyl-2-(3,4-dihydro-1H-isoquinolin-2-yl)-2-oxo-ethyl]-N-(4-pyridin-2-yl-benzyl)-3-(5-trifluoromethyl-pyridin-2-yl)-acrylamide

This compound has been prepared according to the methods of example 1 but using 3-(5-trifluoromethyl-pyridin-2-yl)-acrylic acid for the step 4.

LC-MS: t_{R}=0.96 min; [M+H]⁺=646.89.

### Example 58

### (S)—N-[1-Benzyl-2-(3,4-dihydro-1H-isoquinolin-2-yl)-2-oxo-ethyl]-3-(2-methyl-pyrimidin-5-yl)-N-(4-pyridin-2-yl-benzyl)-acrylamide

This compound has been prepared according to the methods of example 1 but using 3-(2-methyl-pyrimidin-pyridin-5-yl)-acrylic acid for the step 4.

LC-MS: t_{R}=0.87 min; [M+H]⁺=594.16.

### Example 59

### (S)—N-[1-Benzyl-2-(3,4-dihydro-1H-isoquinolin-2-yl)-2-oxo-ethyl]-3-(6-methyl-pyridin-3-yl)-N-(4-pyridin-2-yl-benzyl)-acrylamide

This compound has been prepared according to the methods of example 1 but using 3-(6-methyl-pyridin-pyridin-3-yl)-acrylic acid for the step 4.

LC-MS: t_{R}=0.77 min; [M+H]⁺=593.21.

### Example 60

### (S)—N-[1-Benzyl-2-(3,4-dihydro-1H-isoquinolin-2-yl)-2-oxo-ethyl]-3-(6-chloro-pyridin-3-yl)-N-(4-pyridin-2-yl-benzyl)-acrylamide

This compound has been prepared according to the methods of example 1 but using 3-(6-chloro-pyridin-pyridin-3-yl)-acrylic acid for the step 4.

LC-MS: t_{R}=0.93 min; [M+H]⁺=613.14.

### Example 61

### (S)—N-[1-Benzyl-2-(3,4-dihydro-1H-isoquinolin-2-yl)-2-oxo-ethyl]-3-(5-methyl-pyridin-2-yl)-N-(4-pyridin-2-yl-benzyl)-acrylamide

This compound has been prepared according to the methods of example 1 but using 3-(5-methyl-pyridin-pyridin-2-yl)-acrylic acid for the step 4.

LC-MS: t_{R}=0.83 min; [M+H]⁺=593.19.

### Example 62

### (S)—N-[1-Benzyl-2-(3,4-dihydro-1H-isoquinolin-2-yl)-2-oxo-ethyl]-3-(6-methoxy-pyridin-3-yl)-N-(4-pyridin-2-yl-benzyl)-acrylamide

This compound has been prepared according to the methods of example 1 but using 3-(6-methoxy-pyridin-3-yl)-acrylic acid for the step 4.

LC-MS: t_{R}=0.92 min; [M+H]⁺=609.17.

### Example 63

### (S)—N-[1-Benzyl-2-(3,4-dihydro-1H-isoquinolin-2-yl)-2-oxo-ethyl]-3-(5-methoxy-pyridin-2-yl)-N-(4-pyridin-2-yl-benzyl)-acrylamide

This compound has been prepared according to the methods of example 1 but using 3-(5-methoxy-pyridin-2-yl)-acrylic acid for the step 4.

LC-MS: t_{R}=0.88 min; [M+H]⁺=609.16.

### Example 64

### (S)—N-[1-Benzyl-2-(3,4-dihydro-1H-isoquinolin-2-yl)-2-oxo-ethyl]-N-(4-pyridin-2-yl-benzyl)-3-(2-trifluoromethyl-pyrimidin-5-yl)acrylamide

This compound has been prepared according to the methods of example 1 but using 3-(2-trifluoromethyl-pyrimidin-5-yl)-acrylic acid for the step 4.

LC-MS: t_{R}=0.95 min; [M+H]⁺=648.11.

### Example 65

### (S)—N-[1-Benzyl-2-(3,4-dihydro-1H-isoquinolin-2-yl)-2-oxo-ethyl]-3-(1,5-dimethyl-1H-pyrazol-4-yl)-N-(4-pyridin-2-yl-benzyl)-acrylamide

This compound has been prepared according to the methods of example 1 but using 3-(1,5-dimethyl-1H-pyrazol-4-yl)-acrylic acid for the step 4.

LC-MS: t_{R}=0.88 min; [M+H]⁺=596.19.

Regarding Examples 66 to 87 see further below.

### Example 88

### (S)—N-[1-Benzyl-2-(3,4-dihydro-1-H-isoquinolin-2-yl)-2-oxo-ethyl]-3-(2-fluoro-phenyl)-N-(4-pyridin-2-yl-benzyl)-acrylamide

This compound has been prepared according to the methods of example 1 but using 3-(2-fluoro-phenyl)-acrylic acid for the step 4.

LC-MS: t_{R}=1.05 min; [M+H]⁺=596.11.

### Example 89

### (S)—N-[1-Benzyl-2-(3,4-dihydro-1H-isoquinolin-2-yl)-2-oxo-ethyl]-N-(4-pyrimidin-2-yl-benzyl)-3-(4-trifluoromethyl-phenyl)-acrylamide

This compound has been prepared according to the methods of example 1 but using commercially available 4-pyrimidin-2-yl-benzaldehyde for step 3.

LC-MS: t_{R}=1.15 min; [M+H]⁺=647.15.

### Example 90

### (S)—N-[1-Benzyl-2-(3,4-dihydro-1H-isoquinolin-2-yl)-2-oxo-ethyl]-3-(5-methyl-pyridin-2-yl)-N-(4-pyrimidin-2-yl-benzyl)-acrylamide

This compound has been prepared according to the methods of example 1 but using commercially available 4-pyrimidin-2-yl-benzaldehyde for step 3 and 3-(5-methyl-pyridin-2-yl)-acrylic acid for step 4.

LC-MS: t_{R}=0.96 min; [M+H]⁺=594.18

### Example 91

### (S)—N-[1-Benzyl-2-(3,4-dihydro-1H-isoquinolin-2-yl)-2-oxo-ethyl]-3-(6-methyl-pyridin-3-yl)-N-(4-pyrimidin-2-yl-benzyl)-acrylamide

This compound has been prepared according to the methods of example 1 but using commercially available 4-pyrimidin-2-yl-benzaldehyde for step 3 and 3-(6-methyl-pyridin-3-yl)-acrylic acid for the step 4.

LC-MS: t_{R}=0.92 min; [M+H]⁺=594.83.

### Example 92

### (S)—N-[1-Benzyl-2-(3,4-dihydro-1H-isoquinolin-2-yl)-2-oxo-ethyl]-3-(6-chloro-pyridin-3-yl)-N-(4-pyrimidin-2-yl-benzyl)-acrylamide

This compound has been prepared according to the methods of example 1 but using commercially available 4-pyrimidin-2-yl-benzaldehyde for step 3 and 3-(6-chloro-pyridin-3-yl)-acrylic acid for the step 4.

LC-MS: t_{R}=1.09 min; [M+H]⁺=614.10.

### Example 93

### (S)—N-[1-Benzyl-2-(3,4-dihydro-1H-isoquinolin-2-yl)-2-oxo-ethyl]-3-(6-methoxy-pyridin-3-yl)-N-(4-pyrimidin-2-yl-benzyl)-acrylamide

This compound has been prepared according to the methods of example 1 but using commercially available 4-pyrimidin-2-yl-benzaldehyde for step 3 and 3-(6-methoxy-pyridin-3-yl)-acrylic acid for the step 4.

LC-MS: t_{R}=1.09 min; [M+H]⁺=610.08.

### Example 94

### (S)—N-[1-Benzyl-2-(3,4-dihydro-1H-isoquinolin-2-yl)-2-oxo-ethyl]-3-(2-methyl-pyrimidin-5-yl)-N-(4-pyrimidin-2-yl-benzyl)-acrylamide

This compound has been prepared according to the methods of example 1 but using commercially available 4-pyrimidin-2-yl-benzaldehyde for step 3 and 3-(2-methyl-pyrimidin-5-yl)-acrylic acid for the step 4.

LC-MS: t_{R}=1.07 min; [M+H]⁺=595.17.

### Example 95

### (S)—N-[1-Benzyl-2-(3,4-dihydro-1H-isoquinolin-2-yl)-2-oxo-ethyl]-3-(6-methoxy-pyridazin-3-yl)-N-(4-pyrimidin-2-yl-benzyl)-acrylamide

This compound has been prepared according to the methods of example 1 but using commercially available 4-pyrimidin-2-yl-benzaldehyde for step 3 and 3-(2-methoxy-pyrimidin-5-yl)-acrylic acid for the step 4.

LC-MS: t_{R}=1.09 min; [M+H]⁺=611.60

### Example 96

### (S)—N-[1-Benzyl-2-(3,4-dihydro-1H-isoquinolin-2-yl)-2-oxo-ethyl]-3-(2-methoxy-pyrimidin-5-yl)-N-(4-pyrimidin-2-yl-benzyl)-acrylamide

This compound has been prepared according to the methods of example 1 but using commercially available 4-pyrimidin-2-yl-benzaldehyde for step 3 and 3-(2-methoxy-pyrimidin-5-yl)-acrylic acid for the step 4.

LC-MS: t_{R}=1.08 min; [M+H]⁺=610.88.

### Example 97

### (S)—N-[1-Benzyl-2-(3,4-dihydro-1H-isoquinolin-2-yl)-2-oxo-ethyl]-3-(1-methyl-1H-pyrazol-4-yl)-N-(4-pyrimidin-2-yl-benzyl)-acrylamide

This compound has been prepared according to the methods of example 1 but using commercially available 4-pyrimidin-2-yl-benzaldehyde for step 3 and 3-(1-methyl-1H-pyrazol-4-yl)-acrylic acid for the step 4.

LC-MS: t_{R}=1.04 min; [M+H]⁺=583.16.

### Example 98

### (S)—N-[1-Benzyl-2-(3,4-dihydro-1H-isoquinolin-2-yl)-2-oxo-ethyl]-3-(2,5-dimethyl-2H-pyrazol-3-yl)-N-(4-pyrimidin-2-yl-benzyl)-acrylamide

This compound has been prepared according to the methods of example 1 but using commercially available 4-pyrimidin-2-yl-benzaldehyde for step 3 and 3-(2,5-dimethyl-2H-pyrazol-3-yl)-acrylic acid for the step 4.

LC-MS: t_{R}=1.09 min; [M+H]⁺=597.17.

### Example 99

### (S)—N-[1-Benzyl-2-(3,4-dihydro-1H-isoquinolin-2-yl)-2-oxo-ethyl]-3-(1,5-dimethyl-1H-pyrazol-4-yl)-N-(4-pyrimidin-2-yl-benzyl)-acrylamide

This compound has been prepared according to the methods of example 1 but using commercially available 4-pyrimidin-2-yl-benzaldehyde for step 3 and 3-(1,5-dimethyl-1H-pyrazol-4-yl)-acrylic acid for the step 4.

LC-MS: t_{R}=1.09 min; [M+H]⁺=597.94.

### Example 100

### (S)—N-[1-Benzyl-2-(3,4-dihydro-1H-isoquinolin-2-yl)-2-oxo-ethyl]-N-(4-pyrimidin-2-yl-benzyl)-3-(1,3,5-trimethyl-1H-pyrazol-4-yl)-acrylamide

This compound has been prepared according to the methods of example 1 but using commercially available 4-pyrimidin-2-yl-benzaldehyde for step 3 and 3-(1,3,5-trimethyl-1H-pyrazol-4-yl)-acrylic acid for the step 4.

LC-MS: t_{R}=1.13 min; [M+H]⁺=610.87.

### Example 101

### (S)—N-[1-Benzyl-2-(3,4-dihydro-1H-isoquinolin-2-yl)-2-oxo-ethyl]-3-(2,3-dimethyl-3H-imidazol-4-yl)-N-(4-pyrimidin-2-yl-benzyl)-acrylamide

This compound has been prepared according to the methods of example 1 but using commercially available 4-pyrimidin-2-yl-benzaldehyde for step 3 and 3-(2,3-dimethyl-3H-imidazol-4-yl)-acrylic acid for the step 4.

LC-MS: t_{R}=0.85 min; [M+H]⁺=596.16.

### Example 102

### (S)—N-[1-Benzyl-2-(3,4-dihydro-1H-isoquinolin-2-yl)-2-oxo-ethyl]-3-(3,5-dimethyl-isoxazol-4-yl)-N-(4-pyrimidin-2-yl-benzyl)-acrylamide

This compound has been prepared according to the methods of example 1 but using commercially available 4-pyrimidin-2-yl-benzaldehyde for step 3 and 3-(3,5-dimethyl-isoxazol-4-yl)-acrylic acid for the step 4.

LC-MS: t_{R}=1.08 min; [M+H]⁺=598.16.

### Example 103

### (S)—N-[1-Benzyl-2-(3,4-dihydro-1H-isoquinolin-2-yl)-2-oxo-ethyl]-3-(2,5-dimethyl-oxazol-4-yl)-N-(4-pyrimidin-2-yl-benzyl)-acrylamide

This compound has been prepared according to the methods of example 1 but using commercially available 4-pyrimidin-2-yl-benzaldehyde for step 3 and 3-(2,5-dimethyl-oxazol-4-yl)-acrylic acid for the step 4.

LC-MS: t_{R}=1.05 min; [M+H]⁺=598.16.

### Example 104

### (S)—N-[1-Benzyl-2-(3,4-dihydro-1H-isoquinolin-2-yl)-2-oxo-ethyl]-3-(4-methyl-thiazol-5-yl)-N-(4-pyrimidin-2-yl-benzyl)-acrylamide

This compound has been prepared according to the methods of example 1 but using commercially available 4-pyrimidin-2-yl-benzaldehyde for step 3 and 3-(4-methyl-thiazol-5-yl)-acrylic acid for the step 4.

LC-MS: t_{R}=1.15 min; [M+H]⁺=600.65.

### Example 105

### (S)—N-[1-Benzyl-2-(3,4-dihydro-1H-isoquinolin-2-yl)-2-oxo-ethyl]-3-(2-methyl-thiazol-4-yl)-N-(4-pyrimidin-2-yl-benzyl)-acrylamide

This compound has been prepared according to the methods of example 1 but using commercially available 4-pyrimidin-2-yl-benzaldehyde for step 3 and 3-(2-methyl-thiazol-4-yl)-acrylic acid for the step 4.

LC-MS: t_{R}=1.16 min; [M+H]⁺=600.64.

### Example 106

### (S)—N-[1-Benzyl-2-(3,4-dihydro-1H-isoquinolin-2-yl)-2-oxo-ethyl]-3-(2-methyl-thiazol-5-yl)-N-(4-pyrimidin-2-yl-benzyl)-acrylamide

This compound has been prepared according to the methods of example 1 but using commercially available 4-pyrimidin-2-yl-benzaldehyde for step 3 and 3-(2-methyl-thiazol-5-yl)-acrylic acid for the step 4.

LC-MS: t_{R}=1.06 min; [M+H]⁺=600.10.

### Example 107

### (S)—N-[1-Benzyl-2-(3,4-dihydro-1H-isoquinolin-2-yl)-2-oxo-ethyl]-3-(2,4-dimethyl-thiazol-5-yl)-N-(4-pyrimidin-2-yl-benzyl)-acrylamide

This compound has been prepared according to the methods of example 1 but using commercially available 4-pyrimidin-2-yl-benzaldehyde for step 3 and 3-(2,4-dimethyl-thiazol-5-yl)-acrylic acid for the step 4.

LC-MS: t_{R}=1.13 min; [M+H]⁺=614.14.

### Example 108

### (S)—N-[1-Benzyl-2-(3,4-dihydro-1H-isoquinolin-2-yl)-2-oxo-ethyl]-N-(4-pyrazin-2-yl-benzyl)-3-(4-trifluoromethyl-phenyl)-acrylamide

This compound has been prepared according to the methods of example 1 but using commercially available 4-pyrazin-2-yl-benzaldehyde for step 3.

LC-MS: t_{R}=1.17 min; [M+H]⁺=647.17.

### Example 109

### (S)—N-[1-Benzyl-2-(3,4-dihydro-1H-isoquinolin-2-yl)-2-oxo-ethyl]-3-(5-methyl-pyridin-2-yl)-N-(4-pyrazin-2-yl-benzyl)-acrylamide

This compound has been prepared according to the methods of example 1 but using commercially available 4-pyrazin-2-yl-benzaldehyde for step 3 and 3-(5-methyl-pyridin-2-yl)-acrylic acid for step 4.

LC-MS: t_{R}=0.97 min; [M+H]⁺=594.19.

### Example 110

### (S)—N-[1-Benzyl-2-(3,4-dihydro-1H-isoquinolin-2-yl)-2-oxo-ethyl]-3-(6-methyl-pyridin-3-yl)-N-(4-pyrazin-2-yl-benzyl)-acrylamide

This compound has been prepared according to the methods of example 1 but using commercially available 4-pyrazin-2-yl-benzaldehyde for step 3 and 3-(6-methyl-pyridin-3-yl)-acrylic acid for the step 4.

LC-MS: t_{R}=0.92 min; [M+H]⁺=594.96.

### Example 111

### (S)—N-[1-Benzyl-2-(3,4-dihydro-1H-isoquinolin-2-yl)-2-oxo-ethyl]-3-(6-chloro-pyridin-3-yl)-N-(4-pyrazin-2-yl-benzyl)-acrylamide

This compound has been prepared according to the methods of example 1 but using commercially available 4-pyrazin-2-yl-benzaldehyde for step 3 and 3-(6-chloro-pyridin-3-yl)-acrylic acid for the step 4.

LC-MS: t_{R}=1.11 min; [M+H]⁺=614.07.

### Example 112

### (S)—N-[1-Benzyl-2-(3,4-dihydro-1H-isoquinolin-2-yl)-2-oxo-ethyl]-3-(6-methoxy-pyridin-3-yl)-N-(4-pyrazin-2-yl-benzyl)-acrylamide

This compound has been prepared according to the methods of example 1 but using commercially available 4-pyrazin-2-yl-benzaldehyde for step 3 and 3-(6-methoxy-pyridin-3-yl)-acrylic acid for the step 4.

LC-MS: t_{R}=1.11 min; [M+H]⁺=610.14.

### Example 113

### (S)—N-[1-Benzyl-2-(3,4-dihydro-1H-isoquinolin-2-yl)-2-oxo-ethyl]-3-(2-methyl-pyrimidin-5-yl)-N-(4-pyrazin-2-yl-benzyl)-acrylamide

This compound has been prepared according to the methods of example 1 but using commercially available 4-pyrazin-2-yl-benzaldehyde for step 3 and 3-(2-methyl-pyrimidin-5-yl)-acrylic acid for the step 4.

LC-MS: t_{R}=1.09 min; [M+H]⁺=595.16.

### Example 114

### (S)—N-[1-Benzyl-2-(3,4-dihydro-1H-isoquinolin-2-yl)-2-oxo-ethyl]-3-(2-methoxy-pyrimidin-5-yl)-N-(4-pyrazin-2-yl-benzyl)-acrylamide

This compound has been prepared according to the methods of example 1 but using commercially available 4-pyrazin-2-yl-benzaldehyde for step 3 and 3-(2-methoxy-pyrimidin-5-yl)-acrylic acid for the step 4.

LC-MS: t_{R}=1.10 min; [M+H]⁺=610.87.

### Example 115

### (S)—N-[1-Benzyl-2-(3,4-dihydro-1H-isoquinolin-2-yl)-2-oxo-ethyl]-3-(6-methoxy-pyridazin-3-yl)-N-(4-pyrazin-2-yl-benzyl)-acrylamide

This compound has been prepared according to the methods of example 1 but using commercially available 4-pyrazin-2-yl-benzaldehyde for step 3 and 3-(2-methoxy-pyridazin-5-yl)-acrylic acid for the step 4.

LC-MS: t_{R}=1.09 min; [M+H]⁺=610.86.

### Example 116

### (S)—N-[1-Benzyl-2-(3,4-dihydro-1H-isoquinolin-2-yl)-2-oxo-ethyl]-3-(1-methyl-1H-pyrazol-4-yl)-N-(4-pyrazin-2-yl-benzyl)-acrylamide

This compound has been prepared according to the methods of example 1 but using commercially available 4-pyrazin-2-yl-benzaldehyde for step 3 and 3-(1-methyl-1H-pyrazol-4-yl)-acrylic acid for the step 4.

LC-MS: t_{R}=1.06 min; [M+H]⁺=583.15.

### Example 117

### (S)—N-[1-Benzyl-2-(3,4-dihydro-1H-isoquinolin-2-yl)-2-oxo-ethyl]-3-(2,5-dimethyl-2H-pyrazol-3-yl)-N-(4-pyrazin-2-yl-benzyl)acrylamide

This compound has been prepared according to the methods of example 1 but using commercially available 4-pyrazin-2-yl-benzaldehyde for step 3 and 3-(2,5-dimethyl-2H-pyrazol-3-yl)-acrylic acid for the step 4.

LC-MS: t_{R}=1.14 min; [M+H]⁺=597.83.

### Example 118

### (S)—N-[1-Benzyl-2-(3,4-dihydro-1H-isoquinolin-2-yl)-2-oxo-ethyl]-3-(1,5-dimethyl-1H-pyrazol-4-yl)-N-(4-pyrazin-2-yl-benzyl)-acrylamide

This compound has been prepared according to the methods of example 1 but using commercially available 4-pyrazin-2-yl-benzaldehyde for step 3 and 3-(1,5-dimethyl-1H-pyrazol-4-yl)-acrylic acid for the step 4.

LC-MS: t_{R}=1.07 min; [M+H]⁺=597.17.

### Example 119

### (S)—N-[1-Benzyl-2-(3,4-dihydro-1H-isoquinolin-2-yl)-2-oxo-ethyl]-N-(4-pyrazin-2-yl-benzyl)-3-(1,3,5-trimethyl-1H-pyrazol-4-yl)-acrylamide

This compound has been prepared according to the methods of example 1 but using commercially available 4-pyrazin-2-yl-benzaldehyde for step 3 and 3-(1,3,5-trimethyl-1H-pyrazol-4-yl)-acrylic acid for the step 4.

LC-MS: t_{R}=1.15 min; [M+H]⁺=612.02.

### Example 120

### (S)—N-[1-Benzyl-2-(3,4-dihydro-1H-isoquinolin-2-yl)-2-oxo-ethyl]-3-(2,3-dimethyl-3H-imidazol-4-yl)-N-(4-pyrazin-2-yl-benzyl)-acrylamide

This compound has been prepared according to the methods of example 1 but using commercially available 4-pyrazin-2-yl-benzaldehyde for step 3 and 3-(2,3-dimethyl-3H-imidazol-4-yl)-acrylic acid for the step 4.

LC-MS: t_{R}=0.85 min; [M+H]⁺=597.19.

### Example 121

### (S)—N-[1-Benzyl-2-(3,4-dihydro-1H-isoquinolin-2-yl)-2-oxo-ethyl]-3-(5-methyl-isoxazol-3-yl)-N-(4-pyrazin-2-yl-benzyl)-acrylamide

This compound has been prepared according to the methods of example 1 but using commercially available 4-pyrazin-2-yl-benzaldehyde for step 3 and 3-(5-methyl-isoxazol-3-yl)-acrylic acid for the step 4.

LC-MS: t_{R}=1.08 min; [M+H]⁺=584.15.

### Example 122

### (S)—N-[1-Benzyl-2-(3,4-dihydro-1H-isoquinolin-2-yl)-2-oxo-ethyl]-3-(3,5-dimethyl-isoxazol-4-yl)-N-(4-pyrazin-2-yl-benzyl)acrylamide

This compound has been prepared according to the methods of example 1 but using commercially available 4-pyrazin-2-yl-benzaldehyde for step 3 and 3-(3,5-dimethyl-isoxazol-4-yl)-acrylic acid for the step 4.

LC-MS: t_{R}=1.10 min; [M+H]⁺=598.15.

### Example 123

### (S)—N-[1-Benzyl-2-(3,4-dihydro-1H-isoquinolin-2-yl)-2-oxo-ethyl]-3-(2,5-dimethyl-oxazol-4-yl)-N-(4-pyrazin-2-yl-benzyl)-acrylamide

This compound has been prepared according to the methods of example 1 but using commercially available 4-pyrazin-2-yl-benzaldehyde for step 3 and 3-(2,5-dimethyl-oxazol-4-yl)-acrylic acid for the step 4.

LC-MS: t_{R}=1.07 min; [M+H]⁺=598.16.

### Example 124

### (S)—N-[1-Benzyl-2-(3,4-dihydro-1H-isoquinolin-2-yl)-2-oxo-ethyl]-3-(4-methyl-thiazol-5-yl)-N-(4-pyrazin-2-yl-benzyl)-acrylamide

This compound has been prepared according to the methods of example 1 but using commercially available 4-pyrazin-2-yl-benzaldehyde for step 3 and 3-(4-methyl-thiazol-5-yl)-acrylic acid for the step 4.

LC-MS: t_{R}=1.17 min; [M+H]⁺=600.34.

### Example 125

### (S)—N-[1-Benzyl-2-(3,4-dihydro-1H-isoquinolin-2-yl)-2-oxo-ethyl]-3-(2-methyl-thiazol-4-yl)-N-(4-pyrazin-2-yl-benzyl)-acrylamide

This compound has been prepared according to the methods of example 1 but using commercially available 4-pyrazin-2-yl-benzaldehyde for step 3 and 3-(2-methyl-thiazol-4-yl)-acrylic acid for the step 4.

LC-MS: t_{R}=1.18 min; [M+H]⁺=600.99.

### Example 126

### (S)—N-[1-Benzyl-2-(3,4-dihydro-1H-isoquinolin-2-yl)-2-oxo-ethyl]-3-(2-methyl-thiazol-5-yl)-N-(4-pyrazin-2-yl-benzyl)-acrylamide

This compound has been prepared according to the methods of example 1 but using commercially available 4-pyrazin-2-yl-benzaldehyde for step 3 and 3-(2-methyl-thiazol-5-yl)-acrylic acid for the step 4.

LC-MS: t_{R}=1.08 min; [M+H]⁺=600.11.

### Example 127

### (S)—N-[1-Benzyl-2-(3,4-dihydro-1H-isoquinolin-2-yl)-2-oxo-ethyl]-3-(2,4-dimethyl-thiazol-5-yl)-N-(4-pyrazin-2-yl-benzyl)acrylamide

This compound has been prepared according to the methods of example 1 but using commercially available 4-pyrazin-2-yl-benzaldehyde for step 3 and 3-(2,4-dimethyl-thiazol-5-yl)-acrylic acid for the step 4.

LC-MS: t_{R}=1.15 min; [M+H]⁺=613.94.

### Example 128

### (S)—N-[1-Benzyl-2-(3,4-dihydro-1H-isoquinolin-2-yl)-2-oxo-ethyl]-N-(4-pyridazin-3-yl-benzyl)-3-(4-trifluoromethyl-phenyl)-acrylamide

This compound has been prepared according to the methods of example 1 but using commercially available 4-pyridazin-3-yl-benzaldehyde for step 3.

LC-MS: t_{R}=1.13 min; [M+H]⁺=647.15.

### Example 129

### (S)—N-[1-Benzyl-2-(3,4-dihydro-1H-isoquinolin-2-yl)-2-oxo-ethyl]-3-(5-methyl-pyridin-2-yl)-N-(4-pyridazin-3-yl-benzyl)-acrylamide

This compound has been prepared according to the methods of example 1 but using commercially available 4-pyridazin-3-yl-benzaldehyde for step 3 and 3-(5-methyl-pyridin-2-yl)-acrylic acid for step 4.

LC-MS: t_{R}=0.92 min; [M+H]⁺=594.15.

### Example 130

### (S)—N-[1-Benzyl-2-(3,4-dihydro-1H-isoquinolin-2-yl)-2-oxo-ethyl]-3-(6-methyl-pyridin-3-yl)-N-(4-pyridazin-3-yl-benzyl)-acrylamide

This compound has been prepared according to the methods of example 1 but using commercially available 4-pyridazin-3-yl-benzaldehyde for step 3 and 3-(6-methyl-pyridin-3-yl)-acrylic acid for the step 4.

LC-MS: t_{R}=0.88 min; [M+H]⁺=594.80.

### Example 131

### (S)—N-[1-Benzyl-2-(3,4-dihydro-1H-isoquinolin-2-yl)-2-oxo-ethyl]-3-(6-chloro-pyridin-3-yl)-N-(4-pyridazin-3-yl-benzyl)-acrylamide

This compound has been prepared according to the methods of example 1 but using commercially available 4-pyridazin-3-yl-benzaldehyde for step 3 and 3-(6-chloro-pyridin-3-yl)-acrylic acid for the step 4.

LC-MS: t_{R}=1.06 min; [M+H]⁺=614.

### Example 132

### (S)—N-[1-Benzyl-2-(3,4-dihydro-1H-isoquinolin-2-yl)-2-oxo-ethyl]-3-(6-methoxy-pyridin-3-yl)-N-(4-pyridazin-3-yl-benzyl)-acrylamide

This compound has been prepared according to the methods of example 1 but using commercially available 4-pyridazin-3-yl-benzaldehyde for step 3 and 3-(6-methoxy-pyridin-3-yl)-acrylic acid for the step 4.

LC-MS: t_{R}=1.06 min; [M+H]⁺=610.12.

### Example 133

### (S)—N-[1-Benzyl-2-(3,4-dihydro-1H-isoquinolin-2-yl)-2-oxo-ethyl]-3-(2-methyl-pyrimidin-5-yl)-N-(4-pyridazin-3-yl-benzyl)-acrylamide

This compound has been prepared according to the methods of example 1 but using commercially available 4-pyridazin-3-yl-benzaldehyde for step 3 and 3-(2-methyl-pyrimidin-5-yl)-acrylic acid for the step 4.

LC-MS: t_{R}=1.02 min; [M+H]⁺=595.15.

### Example 134

### (S)—N-[1-Benzyl-2-(3,4-dihydro-1H-isoquinolin-2-yl)-2-oxo-ethyl]-3-(2-methoxy-pyrimidin-5-yl)-N-(4-pyridazin-3-yl-benzyl)-acrylamide

This compound has been prepared according to the methods of example 1 but using commercially available 4-pyridazin-3-yl-benzaldehyde for step 3 and 3-(2-methoxy-pyrimidin-3-yl)-acrylic acid for the step 4.

LC-MS: t_{R}=1.05 min; [M+H]⁺=611.73.

### Example 135

### (S)—N-[1-Benzyl-2-(3,4-dihydro-1H-isoquinolin-2-yl)-2-oxo-ethyl]-3-(6-methoxy-pyridazin-3-yl)-N-(4-pyridazin-3-yl-benzyl)-acrylamide

This compound has been prepared according to the methods of example 1 but using commercially available 4-pyridazin-3-yl-benzaldehyde for step 3 and 3-(6-methoxy-pyridazin-3-yl)-acrylic acid for the step 4.

LC-MS: t_{R}=1.04 min; [M+H]⁺=610.87.

### Example 136

### (S)—N-[1-Benzyl-2-(3,4-dihydro-1H-isoquinolin-2-yl)-2-oxo-ethyl]-3-(1-methyl-1H-pyrazol-4-yl)-N-(4-pyridazin-3-yl-benzyl)-acrylamide

This compound has been prepared according to the methods of example 1 but using commercially available 4-pyridazin-3-yl-benzaldehyde for step 3 and 3-(1-methyl-1H-pyrazol-4-yl)-acrylic acid for the step 4.

LC-MS: t_{R}=0.99 min; [M+H]⁺=583.15.

### Example 137

### (S)—N-[1-Benzyl-2-(3,4-dihydro-1H-isoquinolin-2-yl)-2-oxo-ethyl]-3-(2,5-dimethyl-2H-pyrazol-3-yl)-N-(4-pyridazin-3-yl-benzyl)-acrylamide

This compound has been prepared according to the methods of example 1 but using commercially available 4-pyridazin-3-yl-benzaldehyde for step 3 and 3-(2,5-dimethyl-2H-pyrazol-3-yl)-acrylic acid for the step 4.

LC-MS: t_{R}=1.08 min; [M+H]⁺=596.92.

### Example 138

### (S)—N-[1-Benzyl-2-(3,4-dihydro-1H-isoquinolin-2-yl)-2-oxo-ethyl]-3-(2,3-dimethyl-3H-imidazol-4-yl)-N-(4-pyridazin-3-yl-benzyl)-acrylamide

This compound has been prepared according to the methods of example 1 but using commercially available 4-pyridazin-3-yl-benzaldehyde for step 3 and 3-(2,3-dimethyl-3H-imidazol-4-yl)-acrylic acid for the step 4.

LC-MS: t_{R}=0.82 min; [M+H]⁺=597.16.

### Example 139

### (S)—N-[1-Benzyl-2-(3,4-dihydro-1H-isoquinolin-2-yl)-2-oxo-ethyl]-3-(5-methyl-isoxazol-3-yl)-N-(4-pyridazin-3-yl-benzyl)-acrylamide

This compound has been prepared according to the methods of example 1 but using commercially available 4-pyridazin-3-yl-benzaldehyde for step 3 and 3-(5-methyl-isoxazol-3-yl)-acrylic acid for the step 4.

LC-MS: t_{R}=1.03 min; [M+H]⁺=584.15.

### Example 140

### (S)—N-[1-Benzyl-2-(3,4-dihydro-1H-isoquinolin-2-yl)-2-oxo-ethyl]-3-(3,5-dimethyl-isoxazol-4-yl)-N-(4-pyridazin-3-yl-benzyl)-acrylamide

This compound has been prepared according to the methods of example 1 but using commercially available 4-pyridazin-3-yl-benzaldehyde for step 3 and 3-(3,5-dimethyl-isoxazol-4-yl)-acrylic acid for the step 4.

LC-MS: t_{R}=1.05 min; [M+H]⁺=598.17.

### Example 141

### (S)—N-[1-Benzyl-2-(3,4-dihydro-1H-isoquinolin-2-yl)-2-oxo-ethyl]-3-(2,5-dimethyl-oxazol-4-yl)-N-(4-pyridazin-3-yl-benzyl)-acrylamide

This compound has been prepared according to the methods of example 1 but using commercially available 4-pyridazin-3-yl-benzaldehyde for step 3 and 3-(2,5-dimethyl-oxazol-4-yl)-acrylic acid for the step 4.

LC-MS: t_{R}=1.02 min; [M+H]⁺=598.14.

### Example 142

### (S)—N-[1-Benzyl-2-(3,4-dihydro-1H-isoquinolin-2-yl)-2-oxo-ethyl]-3-(4-methyl-thiazol-5-yl)-N-(4-pyridazin-3-yl-benzyl)-acrylamide

This compound has been prepared according to the methods of example 1 but using commercially available 4-pyridazin-3-yl-benzaldehyde for step 3 and 3-(4-methyl-thiazol-5-yl)-acrylic acid for the step 4.

LC-MS: t_{R}=1.07 min; [M+H]⁺=600.11.

### Example 143

### (S)—N-[1-Benzyl-2-(3,4-dihydro-1H-isoquinolin-2-yl)-2-oxo-ethyl]-3-(2-methyl-thiazol-4-yl)-N-(4-pyridazin-3-yl-benzyl)-acrylamide

This compound has been prepared according to the methods of example 1 but using commercially available 4-pyridazin-3-yl-benzaldehyde for step 3 and 3-(2-methyl-thiazol-4-yl)-acrylic acid for the step 4.

LC-MS: t_{R}=1.07 min; [M+H]⁺=600.13.

### Example 144

### (S)—N-[1-Benzyl-2-(3,4-dihydro-1H-isoquinolin-2-yl)-2-oxo-ethyl]-3-(2-methyl-thiazol-5-yl)-N-(4-pyridazin-3-yl-benzyl)-acrylamide

This compound has been prepared according to the methods of example 1 but using commercially available 4-pyridazin-3-yl-benzaldehyde for step 3 and 3-(2-methyl-thiazol-5-yl)-acrylic acid for the step 4.

LC-MS: t_{R}=1.02 min; [M+H]⁺=600.12.

### Example 145

### (S)—N-[1-Benzyl-2-(3,4-dihydro-1H-isoquinolin-2-yl)-2-oxo-ethyl]-3-(2,4-dimethyl-thiazol-5-yl)-N-(4-pyridazin-3-yl-benzyl)-acrylamide

This compound has been prepared according to the methods of example 1 but using commercially available 4-pyridazin-3-yl-benzaldehyde for step 3 and 3-(2,4-dimethyl-thiazol-5-yl)-acrylic acid for the step 4.

LC-MS: t_{R}=1.08 min; [M+H]⁺=614.16.

### Example 146

### (S)—N-[1-Benzyl-2-(3,4-dihydro-1H-isoquinolin-2-yl)-2-oxo-ethyl]-N-(4-[1,2,3]triazol-1-yl-benzyl)-3-(4-trifluoromethyl-phenyl)-acrylamide

This compound has been prepared according to the methods of example 1 but using commercially available 4-[1,2,3]-triazol-1-yl-benzaldehyde for step 3.

LC-MS: t_{R}=1.16 min; [M+H]⁺=636.43.

### Example 147

### (S)—N-[1-Benzyl-2-(3,4-dihydro-1H-isoquinolin-2-yl)-2-oxo-ethyl]-3-(5-methyl-pyridin-2-yl)-N-(4-[1,2,3]-triazol-1-yl-benzyl)-acrylamide

This compound has been prepared according to the methods of example 1 but using commercially available 4-[1,2,3]-triazol-1-yl-benzaldehyde for step 3 and 3-(5-methyl-pyridin-2-yl)-acrylic acid for step 4.

LC-MS: t_{R}=0.96 min; [M+H]⁺=583.12.

### Example 148

### (S)—N-[1-Benzyl-2-(3,4-dihydro-1H-isoquinolin-2-yl)-2-oxo-ethyl]-3-(6-methyl-pyridin-3-yl)-N-(4-[1,2,3]-triazol-1-yl-benzyl)-acrylamide

This compound has been prepared according to the methods of example 1 but using commercially available 4-[1,2,3]-triazol-1-yl-benzaldehyde for step 3 and 3-(6-methyl-pyridin-3-yl)-acrylic acid for the step 4.

LC-MS: t_{R}=0.89 min; [M+H]⁺=583.06.

### Example 149

### (S)—N-[1-Benzyl-2-(3,4-dihydro-1H-isoquinolin-2-yl)-2-oxo-ethyl]-3-(6-chloro-pyridin-3-yl)-N-(4-[1,2,3]-triazol-1-yl-benzyl)-acrylamide

This compound has been prepared according to the methods of example 1 but using commercially available 4-[1,2,3]-triazol-1-yl-benzaldehyde for step 3 and 3-(6-chloro-pyridin-3-yl)-acrylic acid for the step 4.

LC-MS: t_{R}=1.10 min; [M+H]⁺=602.91.

### Example 150

### (S)—N-[1-Benzyl-2-(3,4-dihydro-1H-isoquinolin-2-yl)-2-oxo-ethyl]-3-(6-methoxy-pyridin-3-yl)-N-(4-[1,2,3]-triazol-1-yl-benzyl)-acrylamide

This compound has been prepared according to the methods of example 1 but using commercially available 4-[1,2,3]-triazol-1-yl-benzaldehyde for step 3 and 3-(6-methoxy-pyridin-3-yl)-acrylic acid for the step 4.

LC-MS: t_{R}=1.10 min; [M+H]⁺=599.01.

### Example 151

### (S)—N-[1-Benzyl-2-(3,4-dihydro-1H-isoquinolin-2-yl)-2-oxo-ethyl]-3-(2-methyl-pyrimidin-5-yl)-N-(4-[1,2,3]-triazol-1-yl-benzyl)-acrylamide

This compound has been prepared according to the methods of example 1 but using commercially available 4-[1,2,3]-triazol-1-yl-benzaldehyde for step 3 and 3-(2-methyl-pyrimidin-5-yl)-acrylic acid for the step 4.

LC-MS: t_{R}=1.06 min; [M+H]⁺=584.

### Example 152

### (S)—N-[1-Benzyl-2-(3,4-dihydro-1H-isoquinolin-2-yl)-2-oxo-ethyl]-3-(2-methoxy-pyrimidin-5-yl)-N-(4-[1,2,3]-triazol-1-yl-benzyl)-acrylamide

This compound has been prepared according to the methods of example 1 but using commercially available 4-[1,2,3]-triazol-1-yl-benzaldehyde for step 3 and 3-(2-methoxy-pyrimidin-5-yl)-acrylic acid for the step 4.

LC-MS: t_{R}=1.05 min; [M+H]⁺=599.95.

### Example 153

### (S)—N-[1-Benzyl-2-(3,4-dihydro-1H-isoquinolin-2-yl)-2-oxo-ethyl]-3-(6-methoxy-pyridazin-3-yl)-N-(4-[1,2,3]-triazol-1-yl-benzyl)-acrylamide

This compound has been prepared according to the methods of example 1 but using commercially available 4-[1,2,3]-triazol-1-yl-benzaldehyde for step 3 and 3-(6-methoxy-pyridazin-3-yl)-acrylic acid for the step 4.

LC-MS: t_{R}=1.04 min; [M+H]⁺=599.98.

### Example 154

### (S)—N-[1-Benzyl-2-(3,4-dihydro-1H-isoquinolin-2-yl)-2-oxo-ethyl]-3-(1-methyl-1H-pyrazol-4-yl)-N-(4-[1,2,3]-triazol-1-yl-benzyl)-acrylamide

This compound has been prepared according to the methods of example 1 but using commercially available 4-[1,2,3]-triazol-1-yl-benzaldehyde for step 3 and 3-(1-methyl-1H-pyrazol-4-yl)-acrylic acid for the step 4.

LC-MS: t_{R}=1.04 min; [M+H]⁺=572.98.

### Example 155

### (S)—N-[1-Benzyl-2-(3,4-dihydro-1H-isoquinolin-2-yl)-2-oxo-ethyl]-3-(2,5-dimethyl-2H-pyrazol-3-yl)-N-(4-[1,2,3]-triazol-1-yl-benzyl)-acrylamide

This compound has been prepared according to the methods of example 1 but using commercially available 4-[1,2,3]-triazol-1-yl-benzaldehyde for step 3 and 3-(2,5-dimethyl-2H-pyrazol-3-yl)-acrylic acid for the step 4.

LC-MS: t_{R}=1.08 min; [M+H]⁺=586.02.

### Example 156

### (S)—N-[1-Benzyl-2-(3,4-dihydro-1H-isoquinolin-2-yl)-2-oxo-ethyl]-3-(1,5-dimethyl-1H-pyrazol-4-yl)-N-(4-[1,2,3]-triazol-1-yl-benzyl)-acrylamide

This compound has been prepared according to the methods of example 1 but using commercially available 4-[1,2,3]-triazol-1-yl-benzaldehyde for step 3 and 3-(1,5-dimethyl-1H-pyrazol-4-yl)-acrylic acid for the step 4.

LC-MS: t_{R}=1.05 min; [M+H]⁺=586.56.

### Example 157

### (S)—N-[1-Benzyl-2-(3,4-dihydro-1H-isoquinolin-2-yl)-2-oxo-ethyl]-N-(4-[1,2,3]-triazol-1-yl-benzyl)-3-(1,3,5-trimethyl-1H-pyrazol-4-yl)-acrylamide

This compound has been prepared according to the methods of example 1 but using commercially available 4-[1,2,3]-triazol-1-yl-benzaldehyde for step 3 and 3-(1,3,5-trimethyl-1H-pyrazol-4-yl)-acrylic acid for the step 4.

LC-MS: t_{R}=1.08 min; [M+H]⁺=600.99.

### Example 158

### (S)—N-[1-Benzyl-2-(3,4-dihydro-1H-isoquinolin-2-yl)-2-oxo-ethyl]-3-(3,5-dimethyl-isoxazol-4-yl)-N-(4-[1,2,3]-triazol-1-yl-benzyl)-acrylamide

This compound has been prepared according to the methods of example 1 but using commercially available 4-[1,2,3]-triazol-1-yl-benzaldehyde for step 3 and 3-(3,5-dimethyl-isoxazol-4-yl)-acrylic acid for the step 4.

LC-MS: t_{R}=1.08 min; [M+H]⁺=587.96.

### Example 159

### (S)—N-[1-Benzyl-2-(3,4-dihydro-1H-isoquinolin-2-yl)-2-oxo-ethyl]-3-(2,5-dimethyl-oxazol-4-yl)-N-(4-[1,2,3]-triazol-1-yl-benzyl)-acrylamide

This compound has been prepared according to the methods of example 1 but using commercially available 4-[1,2,3]-triazol-1-yl-benzaldehyde for step 3 and 3-(2,5-dimethyl-oxazol-4-yl)-acrylic acid for the step 4.

LC-MS: t_{R}=1.06 min; [M+H]⁺=587.04.

### Example 160

### (S)—N-[1-Benzyl-2-(3,4-dihydro-1H-isoquinolin-2-yl)-2-oxo-ethyl]-3-(4-methyl-thiazol-5-yl)-N-(4-[1,2,3]-triazol-1-yl-benzyl)-acrylamide

This compound has been prepared according to the methods of example 1 but using commercially available 4-[1,2,3]-triazol-1-yl-benzaldehyde for step 3 and 3-(4-methyl-thiazol-5-yl)-acrylic acid for the step 4.

LC-MS: t_{R}=1.10 min; [M+H]⁺=589.62.

### Example 161

### (S)—N-[1-Benzyl-2-(3,4-dihydro-1H-isoquinolin-2-yl)-2-oxo-ethyl]-3-(2-methyl-thiazol-4-yl)-N-(4-[1,2,3]-triazol-1-yl-benzyl)-acrylamide

This compound has been prepared according to the methods of example 1 but using commercially available 4-[1,2,3]-triazol-1-yl-benzaldehyde for step 3 and 3-(2-methyl-thiazol-4-yl)-acrylic acid for the step 4.

LC-MS: t_{R}=1.11 min; [M+H]⁺=590.19.

### Example 162

### (S)—N-[1-Benzyl-2-(3,4-dihydro-1H-isoquinolin-2-yl)-2-oxo-ethyl]-3-(2-methyl-thiazol-5-yl)-N-(4-[1,2,3]-triazol-1-yl-benzyl)acrylamide

This compound has been prepared according to the methods of example 1 but using commercially available 4-[1,2,3]-triazol-1-yl-benzaldehyde for step 3 and 3-(2-methyl-thiazol-5-yl)-acrylic acid for the step 4.

LC-MS: t_{R}=1.06 min; [M+H]⁺=589.40.

### Example 163

### (S)—N-[1-Benzyl-2-(3,4-dihydro-1H-isoquinolin-2-yl)-2-oxo-ethyl]-3-(2,4-dimethyl-thiazol-5-yl)-N-(4-[1,2,3]-triazol-1-yl-benzyl)-acrylamide

This compound has been prepared according to the methods of example 1 but using commercially available 4-[1,2,3]-triazol-1-yl-benzaldehyde for step 3 and 3-(2,4-dimethyl-thiazol-5-yl)-acrylic acid for the step 4.

LC-MS: t_{R}=0.94 min; [M+H]⁺=613.15.

### Example 164

### (S)—N-[1-Benzyl-2-(3,4-dihydro-1H-isoquinolin-2-yl)-2-oxo-ethyl]-N-(4-[1,2,4]triazol-1-yl-benzyl)-3-(4-trifluoromethyl-phenyl)-acrylamide

This compound has been prepared according to the methods of example 1 but using commercially available 4-[1,2,4]-triazol-1-yl-benzaldehyde for step 3.

LC-MS: t_{R}=1.18 min; [M+H]⁺=636.57.

### Example 165

### (S)—N-[1-Benzyl-2-(3,4-dihydro-1H-isoquinolin-2-yl)-2-oxo-ethyl]-3-(5-methyl-pyridin-2-yl)-N-(4-[1,2,4]-triazol-1-yl-benzyl)-acrylamide

This compound has been prepared according to the methods of example 1 but using commercially available 4-[1,2,4]-triazol-1-yl-benzaldehyde for step 3 and 3-(5-methyl-pyridin-2-yl)-acrylic acid for step 4.

LC-MS: t_{R}=0.96 min; [M+H]⁺=582.95.

### Example 166

### (S)—N-[1-Benzyl-2-(3,4-dihydro-1H-isoquinolin-2-yl)-2-oxo-ethyl]-3-(6-methyl-pyridin-3-yl)-N-(4-[1,2,4]-triazol-1-yl-benzyl)-acrylamide

This compound has been prepared according to the methods of example 1 but using commercially available 4-[1,2,4]-triazol-1-yl-benzaldehyde for step 3 and 3-(6-methyl-pyridin-3-yl)-acrylic acid for the step 4.

LC-MS: t_{R}=0.88 min; [M+H]⁺=582.92.

### Example 167

### (S)—N-[1-Benzyl-2-(3,4-dihydro-1H-isoquinolin-2-yl)-2-oxo-ethyl]-3-(6-chloro-pyridin-3-yl)-N-(4-[1,2,4]-triazol-1-yl-benzyl)-acrylamide

This compound has been prepared according to the methods of example 1 but using commercially available 4-[1,2,4]-triazol-1-yl-benzaldehyde for step 3 and 3-(6-chloro-pyridin-3-yl)-acrylic acid for the step 4.

LC-MS: t_{R}=1.11 min; [M+H]⁺=602.83.

### Example 168

### (S)—N-[1-Benzyl-2-(3,4-dihydro-1H-isoquinolin-2-yl)-2-oxo-ethyl]-3-(6-methoxy-pyridin-3-yl)-N-(4-[1,2,4]-triazol-1-yl-benzyl)-acrylamide

This compound has been prepared according to the methods of example 1 but using commercially available 4-[1,2,4]-triazol-1-yl-benzaldehyde for step 3 and 3-(6-methoxy-pyridin-3-yl)-acrylic acid for the step 4.

LC-MS: t_{R}=1.11 min; [M+H]⁺=599.99.

### Example 169

### (S)—N-[1-Benzyl-2-(3,4-dihydro-1H-isoquinolin-2-yl)-2-oxo-ethyl]-3-(2-methyl-pyrimidin-5-yl)-N-(4-[1,2,4]-triazol-1-yl-benzyl)-acrylamide

This compound has been prepared according to the methods of example 1 but using commercially available 4-[1,2,4]-triazol-1-yl-benzaldehyde for step 3 and 3-(2-methyl-pyrimidin-5-yl)-acrylic acid for the step 4.

LC-MS: t_{R}=1.06 min; [M+H]⁺=584.05.

### Example 170

### (S)—N-[1-Benzyl-2-(3,4-dihydro-1H-isoquinolin-2-yl)-2-oxo-ethyl]-3-(2-methoxy-pyrimidin-5-yl)-N-(4-[1,2,4]-triazol-1-yl-benzyl)-acrylamide

This compound has been prepared according to the methods of example 1 but using commercially available 4-[1,2,4]-triazol-1-yl-benzaldehyde for step 3 and 3-(2-methoxy-pyrimidin-5-yl)-acrylic acid for the step 4.

LC-MS: t_{R}=1.05 min; [M+H]⁺=600.95.

### Example 171

### (S)—N-[1-Benzyl-2-(3,4-dihydro-1H-isoquinolin-2-yl)-2-oxo-ethyl]-3-(6-methoxy-pyridazin-3-yl)-N-(4-[1,2,4]-triazol-1-yl-benzyl)-acrylamide

This compound has been prepared according to the methods of example 1 but using commercially available 4-[1,2,4]-triazol-1-yl-benzaldehyde for step 3 and 3-(6-methoxy-pyridazin-3-yl)-acrylic acid for the step 4.

LC-MS: t_{R}=1.05 min; [M+H]⁺=600.72.

### Example 172

### (S)—N-[1-Benzyl-2-(3,4-dihydro-1H-isoquinolin-2-yl)-2-oxo-ethyl]-3-(1-methyl-1H-pyrazol-4-yl)-N-(4-[1,2,4]-triazol-1-yl-benzyl)-acrylamide

This compound has been prepared according to the methods of example 1 but using commercially available 4-[1,2,4]-triazol-1-yl-benzaldehyde for step 3 and 3-(1-methyl-1H-pyrazol-4-yl)-acrylic acid for the step 4.

LC-MS: t_{R}=1.04 min; [M+H]⁺=572.56.

### Example 173

### (S)—N-[1-Benzyl-2-(3,4-dihydro-1H-isoquinolin-2-yl)-2-oxo-ethyl]-3-(2,5-dimethyl-2H-pyrazol-3-yl)-N-(4-[1,2,4]-triazol-1-yl-benzyl)-acrylamide

This compound has been prepared according to the methods of example 1 but using commercially available 4-[1,2,4]-triazol-1-yl-benzaldehyde for step 3 and 3-(2,5-dimethyl-2H-pyrazol-3-yl)-acrylic acid for the step 4.

LC-MS: t_{R}=1.08 min; [M+H]⁺=586.75.

### Example 174

### (S)—N-[1-Benzyl-2-(3,4-dihydro-1H-isoquinolin-2-yl)-2-oxo-ethyl]-3-(1,5-dimethyl-1H-pyrazol-4-yl)-N-(4-[1,2,4]-triazol-1-yl-benzyl)-acrylamide

This compound has been prepared according to the methods of example 1 but using commercially available 4-[1,2,4]-triazol-1-yl-benzaldehyde for step 3 and 3-(1,5-dimethyl-1 H-pyrazol-4-yl)-acrylic acid for the step 4.

LC-MS: t_{R}=1.06 min; [M+H]⁺=586.70.

### Example 175

### (S)—N-[1-Benzyl-2-(3,4-dihydro-1H-isoquinolin-2-yl)-2-oxo-ethyl]-N-(4-[1,2,4]-triazol-1-yl-benzyl)-3-(1,3,5-trimethyl-1H-pyrazol-4-yl)-acrylamide

This compound has been prepared according to the methods of example 1 but using commercially available 4-[1,2,4]-triazol-1-yl-benzaldehyde for step 3 and 3-(1,3,5-trimethyl-1H-pyrazol-4-yl)-acrylic acid for the step 4.

LC-MS: t_{R}=1.09 min; [M+H]⁺=601.14.

### Example 176

### (S)—N-[1-Benzyl-2-(3,4-dihydro-1H-isoquinolin-2-yl)-2-oxo-ethyl]-3-(3,5-dimethyl-isoxazol-4-yl)-N-(4-[1,2,4]-triazol-1-yl-benzyl)-acrylamide

This compound has been prepared according to the methods of example 1 but using commercially available 4-[1,2,4]-triazol-1-yl-benzaldehyde for step 3 and 3-(3,5-dimethyl-isoxazol-4-yl)-acrylic acid for the step 4.

LC-MS: t_{R}=1.09 min; [M+H]⁺=587.94.

### Example 177

### (S)—N-[1-Benzyl-2-(3,4-dihydro-1H-isoquinolin-2-yl)-2-oxo-ethyl]-3-(2,5-dimethyl-oxazol-4-yl)-N-(4-[1,2,4]-triazol-1-yl-benzyl)-acrylamide

This compound has been prepared according to the methods of example 1 but using commercially available 4-[1,2,4]-triazol-1-yl-benzaldehyde for step 3 and 3-(2,5-dimethyl-oxazol-4-yl)-acrylic acid for the step 4.

LC-MS: t_{R}=1.06 min; [M+H]⁺=587.74.

### Example 178

### (S)—N-[1-Benzyl-2-(3,4-dihydro-1H-isoquinolin-2-yl)-2-oxo-ethyl]-3-(4-methyl-thiazol-5-yl)-N-(4-[1,2,4]-triazol-1-yl-benzyl)-acrylamide

This compound has been prepared according to the methods of example 1 but using commercially available 4-[1,2,4]-triazol-1-yl-benzaldehyde for step 3 and 3-(4-methyl-thiazol-5-yl)-acrylic acid for the step 4.

LC-MS: t_{R}=1.11 min; [M+H]⁺=589.77.

### Example 179

### (S)—N-[1-Benzyl-2-(3,4-dihydro-1H-isoquinolin-2-yl)-2-oxo-ethyl]-3-(2-methyl-thiazol-4-yl)-N-(4-[1,2,4]-triazol-1-yl-benzyl)-acrylamide

This compound has been prepared according to the methods of example 1 but using commercially available 4-[1,2,4]-triazol-1-yl-benzaldehyde for step 3 and 3-(2-methyl-thiazol-4-yl)-acrylic acid for the step 4.

LC-MS: t_{R}=1.11 min; [M+H]⁺=589.63.

### Example 180

### (S)—N-[1-Benzyl-2-(3,4-dihydro-1H-isoquinolin-2-yl)-2-oxo-ethyl]-3-(2-methyl-thiazol-5-yl)-N-(4-[1,2,4]-triazol-1-yl-benzyl)-acrylamide

This compound has been prepared according to the methods of example 1 but using commercially available 4-[1,2,4]-triazol-1-yl-benzaldehyde for step 3 and 3-(2-methyl-thiazol-5-yl)-acrylic acid for the step 4.

LC-MS: t_{R}=1.07 min; [M+H]⁺=589.07.

### Example 181

### (S)—N-[1-Benzyl-2-(3,4-dihydro-1H-isoquinolin-2-yl)-2-oxo-ethyl]-3-(2,4-dimethyl-thiazol-5-yl)-N-(4-[1,2,4]-triazol-1-yl-benzyl)-acrylamide

This compound has been prepared according to the methods of example 1 but using commercially available 4-[1,2,4]-triazol-1-yl-benzaldehyde for step 3 and 3-(2,4-dimethyl-thiazol-5-yl)-acrylic acid for the step 4.

LC-MS: t_{R}=1.13 min; [M+H]⁺=602.90.

### Example 182

### (S)—N-[1-Benzyl-2-(3,4-dihydro-1H-isoquinolin-2-yl)-2-oxo-ethyl]-N-(4-pyrazol-1-yl-benzyl)-3-(4-trifluoromethyl-phenyl)-acrylamide

This compound has been prepared according to the methods of example 1 but using commercially available 4-pyrazol-1-yl-benzaldehyde for step 3.

LC-MS: t_{R}=1.18 min; [M+H]⁺=635.99.

### Example 183

### (S)—N-[1-Benzyl-2-(3,4-dihydro-1H-isoquinolin-2-yl)-2-oxo-ethyl]-3-(5-methyl-pyridin-2-yl)-N-(4-pyrazol-1-yl-benzyl)-acrylamide

This compound has been prepared according to the methods of example 1 but using commercially available 4-pyrazol-1-yl-benzaldehyde for step 3 and 3-(5-methyl-pyridin-2-yl)-acrylic acid for step 4.

LC-MS: t_{R}=0.99 min; [M+H]⁺=582.49.

### Example 184

### (S)—N-[1-Benzyl-2-(3,4-dihydro-1H-isoquinolin-2-yl)-2-oxo-ethyl]-3-(6-methyl-pyridin-3-yl)-N-(4-pyrazol-1-yl-benzyl)-acrylamide

This compound has been prepared according to the methods of example 1 but using commercially available 4-pyrazol-1-yl-benzaldehyde for step 3 and 3-(6-methyl-pyridin-3-yl)-acrylic acid for the step 4.

LC-MS: t_{R}=0.92 min; [M+H]⁺=582.59.

### Example 185

### (S)—N-[1-Benzyl-2-(3,4-dihydro-1H-isoquinolin-2-yl)-2-oxo-ethyl]-3-(6-chloro-pyridin-3-yl)-N-(4-pyrazol-1-yl-benzyl)-acrylamide

This compound has been prepared according to the methods of example 1 but using commercially available 4-pyrazol-1-yl-benzaldehyde for step 3 and 3-(6-chloro-pyridin-3-yl)-acrylic acid for the step 4.

LC-MS: t_{R}=1.12 min; [M+H]⁺=601.80.

### Example 186

### (S)—N-[1-Benzyl-2-(3,4-dihydro-1H-isoquinolin-2-yl)-2-oxo-ethyl]-3-(6-methoxy-pyridin-3-yl)-N-(4-pyrazol-1-yl-benzyl)-acrylamide

This compound has been prepared according to the methods of example 1 but using commercially available 4-pyrazol-1-yl-benzaldehyde for step 3 and 3-(6-methoxy-pyridin-3-yl)-acrylic acid for the step 4.

LC-MS: t_{R}=1.12 min; [M+H]⁺=599.04.

### Example 187

### (S)—N-[1-Benzyl-2-(3,4-dihydro-1H-isoquinolin-2-yl)-2-oxo-ethyl]-3-(2-methyl-pyrimidin-5-yl)-N-(4-pyrazol-1-yl-benzyl)-acrylamide

This compound has been prepared according to the methods of example 1 but using commercially available 4-pyrazol-1-yl-benzaldehyde for step 3 and 3-(2-methyl-pyrimidin-5-yl)-acrylic acid for the step 4.

LC-MS: t_{R}=1.10 min; [M+H]⁺=582.91.

### Example 188

### (S)—N-[1-Benzyl-2-(3,4-dihydro-1H-isoquinolin-2-yl)-2-oxo-ethyl]-3-(2-methoxy-pyrimidin-5-yl)-N-(4-pyrazol-1-yl-benzyl)-acrylamide

This compound has been prepared according to the methods of example 1 but using commercially available 4-pyrazol-1-yl-benzaldehyde for step 3 and 3-(2-methoxy-pyrimidin-5-yl)-acrylic acid for the step 4.

LC-MS: t_{R}=1.08 min; [M+H]⁺=599.90.

### Example 189

### (S)—N-[1-Benzyl-2-(3,4-dihydro-1H-isoquinolin-2-yl)-2-oxo-ethyl]-3-(6-methoxy-pyridazin-3-yl)-N-(4-pyrazol-1-yl-benzyl)-acrylamide

This compound has been prepared according to the methods of example 1 but using commercially available 4-pyrazol-1-yl-benzaldehyde for step 3 and 3-(6-methoxy-pyridazin-3-yl)-acrylic acid for the step 4.

LC-MS: t_{R}=1.07 min; [M+H]⁺=599.06.

### Example 190

### (S)—N-[1-Benzyl-2-(3,4-dihydro-1H-isoquinolin-2-yl)-2-oxo-ethyl]-3-(1-methyl-1H-pyrazol-4-yl)-N-(4-pyrazol-1-yl-benzyl)-acrylamide

This compound has been prepared according to the methods of example 1 but using commercially available 4-pyrazol-1-yl-benzaldehyde for step 3 and 3-(1-methyl-1H-pyrazol-4-yl)-acrylic acid for the step 4.

LC-MS: t_{R}=1.07 min; [M+H]⁺=571.80.

### Example 191

### (S)—N-[1-Benzyl-2-(3,4-dihydro-1H-isoquinolin-2-yl)-2-oxo-ethyl]-3-(2,5-dimethyl-2H-pyrazol-3-yl)-N-(4-pyrazol-1-yl-benzyl)-acrylamide

This compound has been prepared according to the methods of example 1 but using commercially available 4-pyrazol-1-yl-benzaldehyde for step 3 and 3-(2,5-dimethyl-2H-pyrazol-3-yl)-acrylic acid for the step 4.

LC-MS: t_{R}=1.12 min; [M+H]⁺=585.

### Example 192

### (S)—N-[1-Benzyl-2-(3,4-dihydro-1H-isoquinolin-2-yl)-2-oxo-ethyl]-3-(1,5-dimethyl-1H-pyrazol-4-yl)-N-(4-pyrazol-1-yl-benzyl)acrylamide

This compound has been prepared according to the methods of example 1 but using commercially available 4-pyrazol-1-yl-benzaldehyde for step 3 and 3-(1,5-dimethyl-1H-pyrazol-4-yl)-acrylic acid for the step 4.

LC-MS: t_{R}=1.09 min; [M+H]⁺=584.94.

### Example 193

### (S)—N-[1-Benzyl-2-(3,4-dihydro-1H-isoquinolin-2-yl)-2-oxo-ethyl]-N-(4-pyrazol-1-yl-benzyl)-3-(1,3,5-trimethyl-1H-pyrazol-4-yl)-acrylamide

This compound has been prepared according to the methods of example 1 but using commercially available 4-pyrazol-1-yl-benzaldehyde for step 3 and 3-(1,3,5-trimethyl-1H-pyrazol-4-yl)-acrylic acid for the step 4.

LC-MS: t_{R}=1.11 min; [M+H]⁺=599.82.

### Example 194

### (S)—N-[1-Benzyl-2-(3,4-dihydro-1H-isoquinolin-2-yl)-2-oxo-ethyl]-3-(3,5-dimethyl-isoxazol-4-yl)-N-(4-pyrazol-1-yl-benzyl)-acrylamide

This compound has been prepared according to the methods of example 1 but using commercially available 4-pyrazol-1-yl-benzaldehyde for step 3 and 3-(3,5-dimethyl-isoxazol-4-yl)-acrylic acid for the step 4.

LC-MS: t_{R}=1.11 min; [M+H]⁺=586.82.

### Example 195

### (S)—N-[1-Benzyl-2-(3,4-dihydro-1H-isoquinolin-2-yl)-2-oxo-ethyl]-3-(2,5-dimethyl-oxazol-4-yl)-N-(4-pyrazol-1-yl-benzyl)-acrylamide

This compound has been prepared according to the methods of example 1 but using commercially available 4-pyrazol-1-yl-benzaldehyde for step 3 and 3-(2,5-dimethyl-oxazol-4-yl)-acrylic acid for the step 4.

LC-MS: t_{R}=1.09 min; [M+H]⁺=587.06.

### Example 196

### (S)—N-[1-Benzyl-2-(3,4-dihydro-1H-isoquinolin-2-yl)-2-oxo-ethyl]-3-(4-methyl-thiazol-5-yl)-N-(4-pyrazol-1-yl-benzyl)-acrylamide

This compound has been prepared according to the methods of example 1 but using commercially available 4-pyrazol-1-yl-benzaldehyde for step 3 and 3-(4-methyl-thiazol-5-yl)-acrylic acid for the step 4.

LC-MS: t_{R}=1.14 min; [M+H]⁺=588.96.

### Example 197

### (S)—N-[1-Benzyl-2-(3,4-dihydro-1H-isoquinolin-2-yl)-2-oxo-ethyl]-3-(2-methyl-thiazol-5-yl)-N-(4-pyrazol-1-yl-benzyl)acrylamide

This compound has been prepared according to the methods of example 1 but using commercially available 4-pyrazol-1-yl-benzaldehyde for step 3 and 3-(2-methyl-thiazol-5-yl)-acrylic acid for the step 4.

LC-MS: t_{R}=1.15 min; [M+H]⁺=588.95.

### Example 198

### (S)—N-[1-Benzyl-2-(3,4-dihydro-1H-isoquinolin-2-yl)-2-oxo-ethyl]-3-(2,4-dimethyl-thiazol-5-yl)-N-(4-pyrazol-1-yl-benzyl)-acrylamide

This compound has been prepared according to the methods of example 1 but using commercially available 4-pyrazol-1-yl-benzaldehyde for step 3 and 3-(2,4-dimethyl-thiazol-5-yl)-acrylic acid for the step 4.

LC-MS: t_{R}=1.15 min; [M+H]⁺=602.78.

### Example 199

### (S)—N-[1-Benzyl-2-(3,4-dihydro-1H-isoquinolin-2-yl)-2-oxo-ethyl]-N-(4-thiazol-2-yl-benzyl)-3-(4-trifluoromethyl-phenyl)-acrylamide

This compound has been prepared according to the methods of example 1 but using 4-thiazol-2-yl-benzaldehyde (prepared according to pathway F) for step 3.

LC-MS: t_{R}=1.08 min; [M+H]⁺=652.20.

### Example 200

### (S)—N-[1-Benzyl-2-(3,4-dihydro-1H-isoquinolin-2-yl)-2-oxo-ethyl]-N-(4-thiazol-2-yl-benzyl)-3-(1,3,5-trimethyl-1H-pyrazol-4-yl)-acrylamide

This compound has been prepared according to the methods of example 1 but using 4-thiazol-2-yl-benzaldehyde (prepared according to pathway F) for step 3 and 3-(1,3,5-trimethyl-1H-pyrazol-4-yl)-acrylic acid for the step 4.

LC-MS: t_{R}=0.83 min; [M+H]⁺=616.30.

### Example 201

### (S)—N-[1-Benzyl-2-(3,4-dihydro-1H-isoquinolin-2-yl)-2-oxo-ethyl]-N-(4-thiazol-5-yl-benzyl)-3-(4-trifluoromethyl-phenyl)-acrylamide

This compound has been prepared according to the methods of example 1 but using 4-thiazol-5-yl-benzaldehyde (prepared according to pathway F) for step 3.

LC-MS: t_{R}=1.05 min; [M+H]⁺=652.10.

### Example 202

### (S)—N-[1-Benzyl-2-(3,4-dihydro-1H-isoquinolin-2-yl)-2-oxo-ethyl]-N-(4-thiazol-5-yl-benzyl)-3-(1,3,5-trimethyl-1H-pyrazol-4-yl)-acrylamide

This compound has been prepared according to the methods of example 1 but using 4-thiazol-5-yl-benzaldehyde (prepared according to pathway F) for step 3 and 3-(1,3,5-trimethyl-1H-pyrazol-4-yl)-acrylic acid for the step 4.

LC-MS: t_{R}=0.85 min; [M+H]⁺=616.30.

### Example 203

### (S)—N-[1-Benzyl-2-(3,4-dihydro-1H-isoquinolin-2-yl)-2-oxo-ethyl]-N-(4-oxazol-5-yl-benzyl)-3-(4-trifluoromethyl-phenyl)-acrylamide

This compound has been prepared according to the methods of example 1 but using 4-oxazol-5-yl-benzaldehyde (prepared according to pathway G) for step 3.

LC-MS: t_{R}=1.03 min; [M+H]⁺=636.20.

### Example 204

### (S)—N-[1-Benzyl-2-(3,4-dihydro-1H-isoquinolin-2-yl)-2-oxo-ethyl]-N-(4-oxazol-5-yl-benzyl)-3-(1,3,5-trimethyl-1H-pyrazol-4-yl)-acrylamide

This compound has been prepared according to the methods of example 1 but using 4-oxazol-5-yl-benzaldehyde (prepared according to pathway G) for step 3 and 3-(1,3,5-trimethyl-1H-pyrazol-4-yl)-acrylic acid for the step 4.

LC-MS: t_{R}=0.83 min; [M+H]⁺=600.20.

### Example 205

### (S)—N-[1-Benzyl-2-(3,4-dihydro-1H-isoquinolin-2-yl)-2-oxo-ethyl]-N-[4-(2-methyl-2H-tetrazol-5-yl)-benzyl]-3-(4-trifluoromethyl-phenyl)-acrylamide

This compound has been prepared according to the methods of example 1 but using 4-(2-methyl-2H-tetrazol-5-yl)-benzaldehyde (prepared according to pathway G) for step 3.

LC-MS: t_{R}=0.87 min; [M+H]⁺=651.20.

### Example 206

### (S)—N-[1-Benzyl-2-(3,4-dihydro-1H-isoquinolin-2-yl)-2-oxo-ethyl]-N-[4-(2-methyl-2H-tetrazol-5-yl)-benzyl]-3-(1,3,5-trimethyl-1H-pyrazol-4-yl)-acrylamide

This compound has been prepared according to the methods of example 1 but using 4-(2-methyl-2H-tetrazol-5-yl)-benzaldehyde (prepared according to pathway G) for step 3 and 3-(1,3,5-trimethyl-1H-pyrazol-4-yl)-acrylic acid for the step 4.

LC-MS: t_{R}=0.81 min; [M+H]⁺=615.30.

### Example 207

### (S)—N-[2-(3,4-Dihydro-1H-isoquinolin-2-yl)-2-oxo-1-pyridin-2-ylmethyl-ethyl]-3-(4-methoxy-phenyl)-N-(6-morpholin-4-yl-pyridin-3-ylmethyl)-acrylamide

This compound has been prepared according to the methods of example 1 but using (L)-2-tert-butoxycarbonylamino-3-pyridin-2-yl-propionic acid for the step 1, 6-morpholinonicotinaldehyde for the step 3, and 3-(4-methoxy-phenyl)-acrylic acid for the step 4.

LC-MS: t_{R}=0.78 min; [M+H]⁺=618.23.

### Example 208

### (S)—N-[1-Benzyl-2-(3,4-dihydro-1H-isoquinolin-2-yl)-2-oxo-ethyl]-N-[4-(4-methyl-piperazin-1-yl)-benzyl]-3-(2-methyl-thiazol-4-yl)-acrylamide

This compound has been prepared according to the methods of example 1 but using commercially available 4-(4-methyl-piperazin-1-yl)-benzaldehyde for step 3 and 3-(2-methyl-thiazol-4-yl)-acrylic acid for the step 4.

LC-MS: t_{R}=0.85 min; [M+H]⁺=620.19.

### Example 209

### (S)—N-[1-Benzyl-2-(3,4-dihydro-1H-isoquinolin-2-yl)-2-oxo-ethyl]-3-(3,5-dimethyl-isoxazol-4-yl)-N-[4-(4-methyl-piperazin-1-yl)-benzyl]acrylamide

This compound has been prepared according to the methods of example 1 but using commercially available 4-(4-methyl-piperazin-1-yl)-benzaldehyde for step 3 and 3-(3,5-dimethyl-isoxazol-4-yl)-acrylic acid for the step 4.

LC-MS: t_{R}=0.86 min; [M+H]⁺=618.24.

### Example 210

### (S)—N-[1-Benzyl-2-(3,4-dihydro-1H-isoquinolin-2-yl)-2-oxo-ethyl]-3-(5-chloro-pyridin-2-yl)-N-[4-(4-methyl-piperazin-1-yl)-benzyl]acrylamide

This compound has been prepared according to the methods of example 1 but using commercially available 4-(4-methyl-piperazin-1-yl)-benzaldehyde for step 3 and 3-(5-chloro-pyridin-2-yl)-acrylic acid for the step 4.

LC-MS: t_{R}=0.88 min; [M+H]⁺=634.20.

### Example 211

### (S)—N-[1-Benzyl-2-(3,4-dihydro-1H-isoquinolin-2-yl)-2-oxo-ethyl]-N-[4-(4-methyl-piperazin-1-yl)-benzyl]-3-(5-trifluoromethyl-pyridin-2-yl)-acrylamide

This compound has been prepared according to the methods of example 1 but using commercially available 4-(4-methyl-piperazin-1-yl)-benzaldehyde for step 3 and 3-(5-trifluoromethyl-pyridin-2-yl)-acrylic acid for the step 4.

LC-MS: t_{R}=0.90 min; [M+H]⁺=668.20.

### Example 212

### (S)—N-[1-Benzyl-2-(3,4-dihydro-1H-isoquinolin-2-yl)-2-oxo-ethyl]-3-(6-methoxy-pyridin-3-yl)-N-[4-(4-methyl-piperazin-1-yl)-benzyl]-acrylamide

This compound has been prepared according to the methods of example 1 but using commercially available 4-(4-methyl-piperazin-1-yl)-benzaldehyde for step 3 and 3-(6-methoxy-pyridin-3-yl)-acrylic acid for the step 4.

LC-MS: t_{R}=0.86 min; [M+H]⁺=630.17.

### Example 213

### (S)—N-[1-Benzyl-2-(3,4-dihydro-1H-isoquinolin-2-yl)-2-oxo-ethyl]-N-[4-(4-methyl-piperazin-1-yl)-benzyl]-3-(1,3,5-trimethyl-1H-pyrazol-4-yl)-acrylamide

This compound has been prepared according to the methods of example 1 but using commercially available 4-(4-methyl-piperazin-1-yl)-benzaldehyde for step 3 and 3-(1,3,5-trimethyl-1H-pyrazol-4-yl)-acrylic acid for the step 4.

LC-MS: t_{R}=0.84 min; [M+H]⁺=631.05.

### Example 214

### (S)—N-[1-Benzyl-2-(3,4-dihydro-1H-isoquinolin-2-yl)-2-oxo-ethyl]-3-(1,5-dimethyl-1H-pyrazol-4-yl)-N-[4-(4-methyl-piperazin-1-yl)-benzyl]acrylamide

This compound has been prepared according to the methods of example 1 but using commercially available 4-(4-methyl-piperazin-1-yl)-benzaldehyde for step 3 and 3-(1,5-dimethyl-1H-pyrazol-4-yl)-acrylic acid for the step 4.

LC-MS: t_{R}=0.82 min; [M+H]⁺=617.24.

### Example 215

### (S)—N-[1-Benzyl-2-(3,4-dihydro-1H-isoquinolin-2-yl)-2-oxo-ethyl]-3-(1-methyl-1H-pyrazol-3-yl)-N-(4-morpholin-4-yl-benzyl)-acrylamide

This compound has been prepared according to the methods of example 1 but using commercially available 4-morpholin-4-yl-benzaldehyde for step 3 and 3-(1-methyl-1H-pyrazol-3-yl)-acrylic acid for the step 4.

LC-MS: t_{R}=1.01 min; [M+H]⁺=590.71.

### Example 216

### (S)—N-[1-Benzyl-2-(3,4-dihydro-1H-isoquinolin-2-yl)-2-oxo-ethyl]-3-(5-methyl-isoxazol-3-yl)-N-(4-morpholin-4-yl-benzyl)-acrylamide

This compound has been prepared according to the methods of example 1 but using commercially available 4-morpholin-4-yl-benzaldehyde for step 3 and 3-(5-methyl-isoxazol-3-yl)-acrylic acid for the step 4.

LC-MS: t_{R}=1.06 min; [M+H]⁺=591.62.

### Example 217

### (S)—N-[1-Benzyl-2-(3,4-dihydro-1H-isoquinolin-2-yl)-2-oxo-ethyl]-3-(5-methyl-pyridin-2-yl)-N-(4-morpholin-4-yl-benzyl)-acrylamide

This compound has been prepared according to the methods of example 1 but using commercially available 4-morpholin-4-yl-benzaldehyde for step 3 and 3-(5-methyl-pyridin-2-yl)-acrylic acid for the step 4.

LC-MS: t_{R}=0.95 min; [M+H]⁺=601.12.

### Example 218

### (S)—N-[1-Benzyl-2-(3,4-dihydro-1H-isoquinolin-2-yl)-2-oxo-ethyl]-3-(6-methyl-pyridin-3-yl)-N-(4-morpholin-4-yl-benzyl)-acrylamide

This compound has been prepared according to the methods of example 1 but using commercially available 4-morpholin-4-yl-benzaldehyde for step 3 and 3-(6-methyl-pyridin-3-yl)-acrylic acid for the step 4.

LC-MS: t_{R}=0.88 min; [M+H]⁺=601.84.

### Example 219

### (S)—N-[1-Benzyl-2-(3,4-dihydro-1H-isoquinolin-2-yl)-2-oxo-ethyl]-3-(6-chloro-pyridin-3-yl)-N-(4-morpholin-4-yl-benzyl)-acrylamide

This compound has been prepared according to the methods of example 1 but using commercially available 4-morpholin-4-yl-benzaldehyde for step 3 and 3-(6-chloro-pyridin-3-yl)-acrylic acid for the step 4.

LC-MS: t_{R}=1.09 min; [M+H]⁺=620.99.

### Example 220

### (S)—N-[1-Benzyl-2-(3,4-dihydro-1H-isoquinolin-2-yl)-2-oxo-ethyl]-3-(6-methoxy-pyridin-3-yl)-N-(4-morpholin-4-yl-benzyl)-acrylamide

This compound has been prepared according to the methods of example 1 but using commercially available 4-morpholin-4-yl-benzaldehyde for step 3 and 3-(6-methoxy-pyridin-3-yl)-acrylic acid for the step 4.

LC-MS: t_{R}=1.08 min; [M+H]⁺=617.51.

### Example 221

### (S)—N-[1-Benzyl-2-(3,4-dihydro-1H-isoquinolin-2-yl)-2-oxo-ethyl]-N-(4-morpholin-4-yl-benzyl)-3-(5-trifluoromethyl-pyridin-2-yl)-acrylamide

This compound has been prepared according to the methods of example 1 but using commercially available 4-morpholin-4-yl-benzaldehyde for step 3 and 3-(5-trifluoromethyl-pyridin-2-yl)-acrylic acid for the step 4.

LC-MS: t_{R}=1.11 min; [M+H]⁺=655.85.

### Example 222

### (S)—N-[1-Benzyl-2-(3,4-dihydro-1H-isoquinolin-2-yl)-2-oxo-ethyl]-3-(2-methoxy-pyrimidin-5-yl)-N-(4-morpholin-4-yl-benzyl)-acrylamide

This compound has been prepared according to the methods of example 1 but using commercially available 4-morpholin-4-yl-benzaldehyde for step 3 and 3-(2-methoxy-pyrimidin-5-yl)-acrylic acid for the step 4.

LC-MS: t_{R}=1.04 min; [M+H]⁺=618.71.

### Example 223

### (S)—N-[1-Benzyl-2-(3,4-dihydro-1H-isoquinolin-2-yl)-2-oxo-ethyl]-3-(2,5-dimethyl-2H-pyrazol-3-yl)-N-(4-morpholin-4-yl-benzyl)-acrylamide

This compound has been prepared according to the methods of example 1 but using commercially available 4-morpholin-4-yl-benzaldehyde for step 3 and 3-(2,5-dimethyl-2H-pyrazol-3-yl)-acrylic acid for the step 4.

LC-MS: t_{R}=1.08 min; [M+H]⁺=604.82.

### Example 224

### (S)—N-[1-Benzyl-2-(3,4-dihydro-1H-isoquinolin-2-yl)-2-oxo-ethyl]-3-(3,5-dimethyl-isoxazol-4-yl)-N-(4-morpholin-4-yl-benzyl)-acrylamide

This compound has been prepared according to the methods of example 1 but using commercially available 4-morpholin-4-yl-benzaldehyde for step 3 and 3-(3,5-dimethyl-isoxazol-4-yl)-acrylic acid for the step 4.

LC-MS: t_{R}=1.07 min; [M+H]⁺=605.47.

### Example 225

### (S)—N-[1-Benzyl-2-(3,4-dihydro-1H-isoquinolin-2-yl)-2-oxo-ethyl]-3-(2,5-dimethyl-oxazol-4-yl)-N-(4-morpholin-4-yl-benzyl)-acrylamide

This compound has been prepared according to the methods of example 1 but using commercially available 4-morpholin-4-yl-benzaldehyde for step 3 and 3-(2,5-dimethyl-oxazol-4-yl)-acrylic acid for the step 4.

LC-MS: t_{R}=1.04 min; [M+H]⁺=605.58.

### Example 226

### (S)—N-[1-Benzyl-2-(3,4-dihydro-1H-isoquinolin-2-yl)-2-oxo-ethyl]-3-(2,3-dimethyl-3H-imidazol-4-yl)-N-(4-morpholin-4-yl-benzyl)-acrylamide

This compound has been prepared according to the methods of example 1 but using commercially available 4-morpholin-4-yl-benzaldehyde for step 3 and 3-(2,3-dimethyl-3H-imidazol-4-yl)-acrylic acid for the step 4.

LC-MS: t_{R}=0.85 min; [M+H]⁺=604.39.

### Example 227

### (S)—N-[1-Benzyl-2-(3,4-dihydro-1H-isoquinolin-2-yl)-2-oxo-ethyl]-3-(5-methyl-pyridin-2-yl)-N-(4-pyrimidin-5-yl-benzyl)-acrylamide

This compound has been prepared according to the methods of example 1 but using commercially available 4-pyrimidin-5-yl-benzaldehyde for step 3 and 3-(5-methyl-pyridin-2-yl)-acrylic acid for the step 4.

LC-MS: t_{R}=0.97 min; [M+H]⁺=594.17.

### Example 228

### (S)—N-[1-Benzyl-2-(3,4-dihydro-1H-isoquinolin-2-yl)-2-oxo-ethyl]-3-(6-chloro-pyridin-3-yl)-N-(4-pyrimidin-5-yl-benzyl)-acrylamide

This compound has been prepared according to the methods of example 1 but using commercially available 4-pyridimin-5-yl-benzaldehyde for step 3 and 3-(6-chloro-pyridin-3-yl)-acrylic acid for the step 4.

LC-MS: t_{R}=1.14 min; [M+H]⁺=614.12.

### Example 229

### (S)—N-[1-Benzyl-2-(3,4-dihydro-1H-isoquinolin-2-yl)-2-oxo-ethyl]-3-(6-methoxy-pyridin-3-yl)-N-(4-pyrimidin-5-yl-benzyl)-acrylamide

This compound has been prepared according to the methods of example 1 but using commercially available 4-pyrimidin-5-yl-benzaldehyde for step 3 and 3-(6-methoxy-pyridin-3-yl)-acrylic acid for the step 4.

LC-MS: t_{R}=1.14 min; [M+H]⁺=610.11.

### Example 230

### (S)—N-[1-Benzyl-2-(3,4-dihydro-1H-isoquinolin-2-yl)-2-oxo-ethyl]-N-(4-pyrimidin-5-yl-benzyl)-3-(5-trifluoromethyl-pyridin-2-yl)-acrylamide

This compound has been prepared according to the methods of example 1 but using commercially available 4-pyrimidin-5-yl-benzaldehyde for step 3 and 3-(5-trifluoromethyl-pyridin-2-yl)-acrylic acid for the step 4.

LC-MS: t_{R}=1.16 min; [M+H]⁺=648.14.

### Example 231

### (S)—N-[1-Benzyl-2-(3,4-dihydro-1H-isoquinolin-2-yl)-2-oxo-ethyl]-3-(2-methyl-pyrimidin-5-yl)-N-(4-pyrimidin-5-yl-benzyl)-acrylamide

This compound has been prepared according to the methods of example 1 but using commercially available 4-pyrimidin-5-yl-benzaldehyde for step 3 and 3-(2-methyl-pyrimidin-5-yl)-acrylic acid for the step 4.

LC-MS: t_{R}=1.10 min; [M+H]⁺=595.17.

### Example 232

### (S)—N-[1-Benzyl-2-(3,4-dihydro-1H-isoquinolin-2-yl)-2-oxo-ethyl]-3-(2,4-dimethyl-thiazol-5-yl)-N-(4-pyrimidin-5-yl-benzyl)-acrylamide

This compound has been prepared according to the methods of example 1 but using commercially available 4-pyrimidin-5-yl-benzaldehyde for step 3 and 3-(2,4-dimethyl-thiazol-5-yl)-acrylic acid for the step 4.

LC-MS: t_{R}=1.18 min; [M+H]⁺=614.13.

### Example 233

### (S)—N-[1-Benzyl-2-(3,4-dihydro-1H-isoquinolin-2-yl)-2-oxo-ethyl]-3-(2-methyl-thiazol-4-yl)-N-(4-pyrimidin-5-yl-benzyl)-acrylamide

This compound has been prepared according to the methods of example 1 but using commercially available 4-pyrimidin-5-yl-benzaldehyde for step 3 and 3-(2-methyl-thiazol-4-yl)-acrylic acid for the step 4.

LC-MS: t_{R}=1.10 min; [M+H]⁺=600.12.

### Example 234

### (S)—N-[1-Benzyl-2-(3,4-dihydro-1H-isoquinolin-2-yl)-2-oxo-ethyl]-3-(5-methyl-isoxazol-3-yl)-N-(4-pyrimidin-5-yl-benzyl)-acrylamide

This compound has been prepared according to the methods of example 1 but using commercially available 4-pyrimidin-5-yl-benzaldehyde for step 3 and 3-(5-methyl-isoxazol-3-yl)-acrylic acid for the step 4.

LC-MS: t_{R}=1.10 min; [M+H]⁺=584.17.

### Example 235

### (S)—N-[1-Benzyl-2-(3,4-dihydro-1H-isoquinolin-2-yl)-2-oxo-ethyl]-3-(3,5-dimethyl-isoxazol-4-yl)-N-(4-pyrimidin-5-yl-benzyl)-acrylamide

This compound has been prepared according to the methods of example 1 but using commercially available 4-pyrimidin-5-yl-benzaldehyde for step 3 and 3-(3,5-dimethyl-isoxazol-4-yl)-acrylic acid for the step 4.

LC-MS: t_{R}=1.13 min; [M+H]⁺=598.14.

### Example 236

### (S)—N-[1-Benzyl-2-(3,4-dihydro-1H-isoquinolin-2-yl)-2-oxo-ethyl]-3-(5-methoxy-pyridin-2-yl)-N-(4-pyridin-4-yl-benzyl)-acrylamide

This compound has been prepared according to the methods of example 1 but using commercially available 4-pyridin-4-yl-benzaldehyde for step 3 and 3-(5-methoxy-pyridin-2-yl)-acrylic acid for the step 4.

LC-MS: t_{R}=0.94 min; [M+H]⁺=608.98.

### Example 237

### (S)—N-[1-Benzyl-2-(3,4-dihydro-1H-isoquinolin-2-yl)-2-oxo-ethyl]-3-(5-methyl-pyridin-2-yl)-N-(4-pyridin-4-yl-benzyl)-acrylamide

This compound has been prepared according to the methods of example 1 but using commercially available 4-pyridin-4-yl-benzaldehyde for step 3 and 3-(5-methyl-pyridin-2-yl)-acrylic acid for the step 4.

LC-MS: t_{R}=0.79 min; [M+H]⁺=593.19.

### Example 238

### (S)—N-[1-Benzyl-2-(3,4-dihydro-1H-isoquinolin-2-yl)-2-oxo-ethyl]-3-(2-methyl-pyrimidin-5-yl)-N-(4-pyridin-4-yl-benzyl)-acrylamide

This compound has been prepared according to the methods of example 1 but using commercially available 4-pyridin-4-yl-benzaldehyde for step 3 and 3-(2-methyl-pyrimidin-5-yl)-acrylic acid for the step 4.

LC-MS: t_{R}=0.83 min; [M+H]⁺=594.21.

### Example 239

### (S)—N-[1-Benzyl-2-(3,4-dihydro-1H-isoquinolin-2-yl)-2-oxo-ethyl]-3-(6-methoxy-pyridazin-3-yl)-N-(4-pyridin-4-yl-benzyl)-acrylamide

This compound has been prepared according to the methods of example 1 but using commercially available 4-pyridin-4-yl-benzaldehyde for step 3 and 3-(6-methoxy-pyridazin-3-yl)-acrylic acid for the step 4.

LC-MS: t_{R}=0.84 min; [M+H]⁺=610.2.

### Example 240

### (S)—N-[1-Benzyl-2-(3,4-dihydro-1H-isoquinolin-2-yl)-2-oxo-ethyl]-N-(4-pyridin-4-yl-benzyl)-3-[1,2,3]thiadiazol-4-yl-acrylamide

This compound has been prepared according to the methods of example 1 but using commercially available 4-pyridin-4-yl-benzaldehyde for step 3 and 3-[1,2,3]thiadiazol-4-yl-acrylic acid for the step 4.

LC-MS: t_{R}=0.85 min; [M+H]⁺=586.12.

### Example 241

### (S)—N-[1-Benzyl-2-(3,4-dihydro-1H-isoquinolin-2-yl)-2-oxo-ethyl]-3-(3,5-dimethyl-isoxazol-4-yl)-N-(4-pyridin-4-yl-benzyl)-acrylamide

This compound has been prepared according to the methods of example 1 but using commercially available 4-pyridin-4-yl-benzaldehyde for step 3 and 3-(3,5-dimethyl-isoxazol-4-yl)-acrylic acid for the step 4.

LC-MS: t_{R}=0.88 min; [M+H]⁺=597.18.

### Example 242

### (S)—N-[1-Benzyl-2-(3,4-dihydro-1H-isoquinolin-2-yl)-2-oxo-ethyl]-3-(2,5-dimethyl-oxazol-4-yl)-N-(4-pyridin-4-yl-benzyl)-acrylamide

This compound has been prepared according to the methods of example 1 but using commercially available 4-pyridin-4-yl-benzaldehyde for step 3 and 3-(2,5-dimethyl-oxazol-4-yl)-acrylic acid for the step 4.

LC-MS: t_{R}=0.86 min; [M+H]⁺=597.18.

### Example 243

### (S)—N-[1-Benzyl-2-(3,4-dihydro-1H-isoquinolin-2-yl)-2-oxo-ethyl]-3-(1-methyl-1H-pyrazol-3-yl)-N-(4-pyridin-4-yl-benzyl)-acrylamide

This compound has been prepared according to the methods of example 1 but using commercially available 4-pyridin-4-yl-benzaldehyde for step 3 and 3-(1-methyl-1H-pyrazol-3-yl)-acrylic acid for the step 4.

LC-MS: t_{R}=0.83 min; [M+H]⁺=582.19.

### Example 244

### (S)—N-[1-Benzyl-2-(3,4-dihydro-1H-isoquinolin-2-yl)-2-oxo-ethyl]-3-(2,5-dimethyl-2H-pyrazol-3-yl)-N-(4-pyridin-4-yl-benzyl)-acrylamide

This compound has been prepared according to the methods of example 1 but using commercially available 4-pyridin-4-yl-benzaldehyde for step 3 and 3-(2,5-dimethyl-2H-pyrazol-3-yl)-acrylic acid for the step 4.

LC-MS: t_{R}=0.86 min; [M+H]⁺=595.29.

### Example 245

### (S)—N-[1-Benzyl-2-(3,4-dihydro-1H-isoquinolin-2-yl)-2-oxo-ethyl]-3-(1,3,5-trimethyl-1H-pyrazol-4-yl)-N-(4-pyridin-4-yl-benzyl)-acrylamide

This compound has been prepared according to the methods of example 1 but using commercially available 4-pyridin-4-yl-benzaldehyde for step 3 and 3-(1,3,5-trimethyl-1H-pyrazol-4-yl)-acrylic acid for the step 4.

LC-MS: t_{R}=0.86 min; [M+H]⁺=610.20.

### Example 246

### (S)—N-[1-Benzyl-2-(3,4-dihydro-1H-isoquinolin-2-yl)-2-oxo-ethyl]-3-(2,3-dimethyl-3H-imidazol-4-yl)-N-(4-pyridin-4-yl-benzyl)-acrylamide

This compound has been prepared according to the methods of example 1 but using commercially available 4-pyridin-4-yl-benzaldehyde for step 3 and 3-(2,3-dimethyl-3H-imidazol-4-yl)-acrylic acid for the step 4.

LC-MS: t_{R}=0.71 min; [M+H]⁺=596.18.

### Example 247

### (S)—N-[1-Benzyl-2-(3,4-dihydro-1H-isoquinolin-2-yl)-2-oxo-ethyl]-3-(2,4-dimethyl-thiazol-5-yl)-N-(4-pyridin-4-yl-benzyl)-acrylamide

This compound has been prepared according to the methods of example 1 but using commercially available 4-pyridin-4-yl-benzaldehyde for step 3 and 3-(2,4-dimethyl-thiazol-5-yl)-acrylic acid for the step 4.

LC-MS: t_{R}=0.88 min; [M+H]⁺=613.14.

### Example 248

### (S)—N-[1-Benzyl-2-(3,4-dihydro-1H-isoquinolin-2-yl)-2-oxo-ethyl]-3-(2-methyl-thiazol-4-yl)-N-(4-pyridin-4-yl-benzyl)-acrylamide

This compound has been prepared according to the methods of example 1 but using commercially available 4-pyridin-4-yl-benzaldehyde for step 3 and 3-(2-methyl-thiazol-4-yl)-acrylic acid for the step 4.

LC-MS: t_{R}=0.86 min; [M+H]⁺=599.13.

### Example 249

### (S)—N-[1-Benzyl-2-(3,4-dihydro-1H-isoquinolin-2-yl)-2-oxo-ethyl]-N-(4-pyridin-4-yl-benzyl)-3-(5-trifluoromethyl-pyridin-2-yl)-acrylamide

This compound has been prepared according to the methods of example 1 but using commercially available 4-pyridin-4-yl-benzaldehyde for step 3 and 3-(5-trifluoromethyl-pyridin-2-yl)-acrylic acid for the step 4.

LC-MS: t_{R}=0.92 min; [M+H]⁺=647.18.

### Example 250

### (S)—N-[1-Benzyl-2-(3,4-dihydro-1H-isoquinolin-2-yl)-2-oxo-ethyl]-3-(1,5-dimethyl-1H-pyrazol-4-yl)-N-(4-pyridin-4-yl-benzyl)-acrylamide

This compound has been prepared according to the methods of example 1 but using commercially available 4-pyridin-4-yl-benzaldehyde for step 3 and 3-(1,5-dimethyl-1H-pyrazol-4-yl)-acrylic acid for the step 4.

LC-MS: t_{R}=0.85 min; [M+H]⁺=596.21.

### Example 251

### (S)—N-[1-Benzyl-2-(3,4-dihydro-1H-isoquinolin-2-yl)-2-oxo-ethyl]-3-(2,4-dimethyl-thiazol-5-yl)-N-(4-morpholin-4-yl-benzyl)-acrylamide

This compound has been prepared according to the methods of example 1 but using commercially available 4-morpholin-4-yl-benzaldehyde for step 3 and 3-(2,4-dimethyl-thiazol-5-yl)-acrylic acid for the step 4.

LC-MS: t_{R}=1.11 min; [M+H]⁺=621.72.

### Example 252

### (S)—N-[1-Benzyl-2-(3,4-dihydro-1H-isoquinolin-2-yl)-2-oxo-ethyl]-3-(2-methyl-thiazol-4-yl)-N-(4-morpholin-4-yl-benzyl)-acrylamide

This compound has been prepared according to the methods of example 1 but using commercially available 4-morpholin-4-yl-benzaldehyde for step 3 and 3-(2-methyl-thiazol-4-yl)-acrylic acid for the step 4.

LC-MS: t_{R}=1.05 min; [M+H]⁺=607.33.

### Example 253

### (S)—N-[1-Benzyl-2-(3,4-dihydro-1H-isoquinolin-2-yl)-2-oxo-ethyl]-3-(2,5-dimethyl-oxazol-4-yl)-N-(4-pyrimidin-5-yl-benzyl)-acrylamide

This compound has been prepared according to the methods of example 1 but using commercially available 4-pyrimidin-5-yl-benzaldehyde for step 3 and 3-(2,5-dimethyl-oxazol-4-yl)-acrylic acid for the step 4.

LC-MS: t_{R}=1.09 min; [M+H]⁺=598.18.

### Example 254

### (S)—N-[1-Benzyl-2-(3,4-dihydro-1-H-isoquinolin-2-yl)-2-oxo-ethyl]-N-[4-(1-oxy-pyridin-4-yl)-benzyl]-3-(4-trifluoromethyl-phenyl)-acrylamide

This compound has been prepared by oxidation of example 39 with m-CPBA (1 equivalent) in an aprotic solvent such as DCM.

LC-MS: t_{R}=0.94 min; [M+H]⁺=662.05.

### Preparation of Compound of Formula I Via Method B

### Example 66

### Step 1: (S)-3-Phenyl-2-(4-pyridin-2-yl-benzylamino)-propionic acid methyl ester

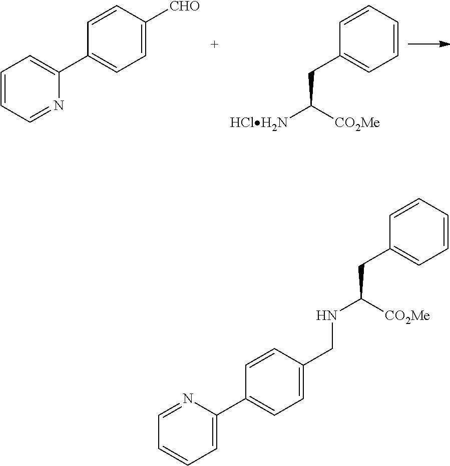

To a solution of L-phenylalanine-methylester hydrochloride (2.4 g, 11.13 mmol), and TEA (1.55 mL, 11.13 mmol) in dry MeOH (5 mL) was added in one portion 4-(2-pyridyl)-benzaldehyde (2.04 g, 11.13 mmol). The reaction was stirred at reflux for 12 h and cooled to rt. Then was added in several portions sodium borohydride (631.5 mg, 16.7 mmol), the mixture was stirred at rt for 1 h, quenched into sat. NaHCO₃ solution, and extracted with EA. The combined organic extracts were washed with brine, dried (MgSO₄), filtered and concentrated to yield the crude title compound (3.8 g, 99%) which was used for the next step without further purification.

LC-MS: t_{R}=0.70 min; [M+H]⁺=347.40.

### Step 2

### a) Trans-3-(4-trifluoromethyl-phenyl)acryloyl chloride

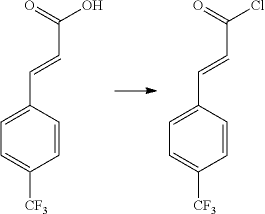

To a cold (0° C.) solution of trans-4-trifluoromethylcinnamic acid (6 g) in a mixture of DCM (20 mL) and DMF (few drops) was added dropwise oxalyl chloride (2.6 mL, 1.1 equivalents). The reaction mixture was stirred at 0° C. for 3 h and concentrated in vacuo to yield the crude title compound as a beige solid (6.5 g, 99%).

### b) (S)-3-Phenyl-2-{(4-pyridin-2-yl-benzyl)-[3-(4-trifluoromethyl-phenyl)-acryloyl]-amino}-propionic acid methyl ester

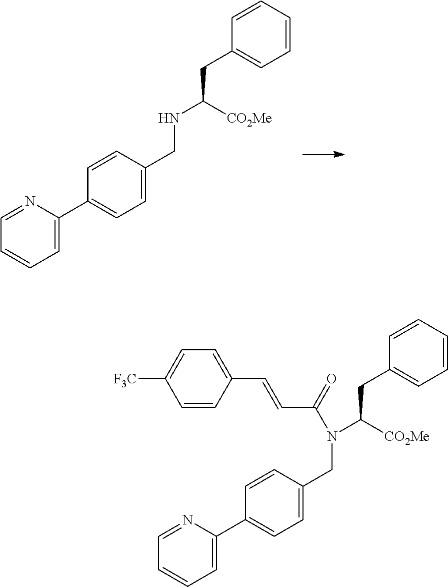

To a cold (0° C.) solution of (S)-3-phenyl-2-(4-pyridin-2-yl-benzylamino)-propionic acid methyl ester (3.6 g, 10.4 mmol), and DIPEA (5.35 mL, 31.17 mmol) in dry DCM (40 mL) was added trans-3-(4-trifluoromethyl-phenyl)-acryloyl chloride (4.87 g, 20.78 mmol). The reaction mixture was stirred at 0° C. for 20 min and then concentrated in vacuo. The resulting residue was taken up in EA, washed with brine, dried (MgSO₄), filtered and concentrated to yield a crude brown oil.

FC (EA/n-heptane: 1/1) gave the title compound as a yellow oil (3.45 g, 61%).

LC-MS: t_{R}=0.97 min; [M+H]⁺=545.31.

### Step 3: (S)-3-Phenyl-2-{(4-pyridin-2-yl-benzyl)-[3-(4-trifluoromethyl-phenyl)-acryloyl]-amino}-propionic acid

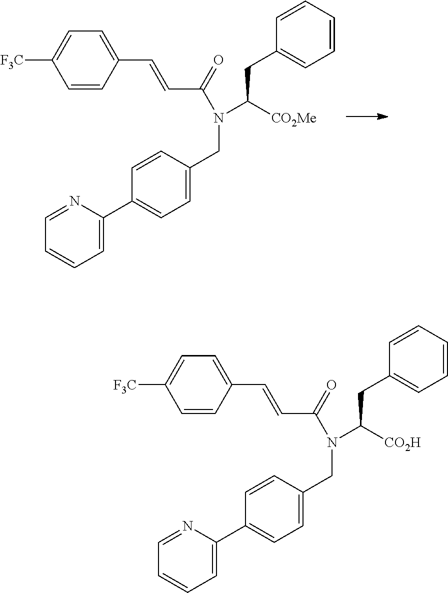

To a solution of (S)-3-phenyl-2-{(4-pyridin-2-yl-benzyl)-[3-(4-trifluoromethyl-phenyl)-acryloyl]-amino}-propionic acid methyl ester (3.45 g, 6.33 mmol) in MeOH (30 mL) was added dropwise aq. NaOH 1N (26 mL, 25.34 mmol). The reaction mixture was stirred at rt for 3 h, then water (30 mL) was added and methanol was evaporated in vacuo. The residue was acidified with aq. HCl 1N until pH<6. Solid NaCl was added until the aq. phase was saturated and then extracted with EA. The combined organic extracts were dried (MgSO₄), filtered and concentrated to yield the title compound as yellow foam (3.43 g, 100%).

LC-MS: t_{R}=0.91 min; [M+H]⁺=531.23.

### Step 4: (S)—N-[1-Benzyl-2-oxo-2-(6-trifluoromethyl-3,4-dihydro-1H-isoquinolin-2-yl)-ethyl]-N-(4-pyridin-2-yl-benzyl)-3-(4-trifluoromethyl-phenyl)-acrylamide

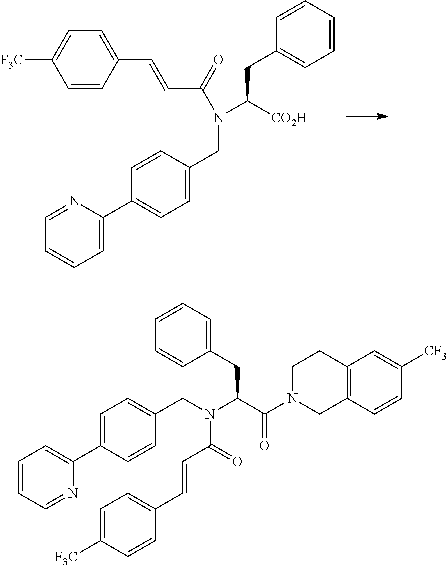

A mixture of (S)-3-phenyl-2-{(4-pyridin-2-yl-benzyl)-[3-(4-trifluoromethyl-phenyl)-acryloyl]-amino}propionic acid (50 mg, 0.094 mmol), TBTU (30.3 mg, 0.094 mmol), and DIPEA (0.08 mL, 0.471 mmol) in dry DMF (1 mL) was stirred at rt for 30 min. Then a solution of 6-trifluoromethyl-1,2,3,4-tetrahydro-isoquinoline hydrochloride in dry DMF (1 mL) was added and the reaction mixture was stirred overnight at rt and directly purified by preparative HPLC.

LC-MS: t_{R}=1.04 min; [M+H]⁺=714.15.

### Example 67

### (S)—N-[1-Benzyl-2-oxo-2-(7-trifluoromethyl-3,4-dihydro-1H-isoquinolin-2-yl)-ethyl]-N-(4-pyridin-2-yl-benzyl)-3-(4-trifluoromethyl-phenyl)-acrylamide

This compound has been prepared according to the methods of example 66 but using 6-trifluoromethyl-1,2,3,4-tetrahydro-isoquinoline hydrochloride for the final step.

LC-MS: t_{R}=1.05 min; [M+H]⁺=714.15.

### Example 68

### (S)—N-[1-Benzyl-2-(7-chloro-3,4-dihydro-1H-isoquinolin-2-yl)-2-oxo-ethyl]-N-(4-pyridin-2-yl-benzyl)-3-(4-trifluoromethyl-phenyl)-acrylamide

This compound has been prepared according to the methods of example 66 but using 7-chloro-1,2,3,4-tetrahydro-isoquinoline hydrochloride for the final step.

LC-MS: t_{R}=1.02 min; [M+H]⁺=680.20.

### Example 69

### (S)—N-[1-Benzyl-2-(7-methoxy-3,4-dihydro-1H-isoquinolin-2-yl)-2-oxo-ethyl]-N-(4-pyridin-2-yl-benzyl)-3-(4-trifluoromethyl-phenyl)-acrylamide

This compound has been prepared according to the methods of example 66 but using 7-methoxy-1,2,3,4-tetrahydro-isoquinoline hydrochloride for the final step.

LC-MS: t_{R}=1.00 min; [M+H]⁺=676.25.

### Example 70

### (S)—N-[1-Benzyl-2-(7-fluoro-3,4-dihydro-1H-isoquinolin-2-yl)-2-oxo-ethyl]-N-(4-pyridin-2-yl-benzyl)-3-(4-trifluoromethyl-phenyl)-acrylamide

This compound has been prepared according to the methods of example 66 but using 7-fluoro-1,2,3,4-tetrahydro-isoquinoline hydrochloride for the final step.

LC-MS: t_{R}=1.01 min; [M+H]⁺=664.20.

### Example 71

### (S)—N-[1-Benzyl-2-(8-chloro-3,4-dihydro-1H-isoquinolin-2-yl)-2-oxo-ethyl]-N-(4-pyridin-2-yl-benzyl)-3-(4-trifluoromethyl-phenyl)-acrylamide

This compound has been prepared according to the methods of example 66 but using 8-chloro-1,2,3,4-tetrahydro-isoquinoline hydrochloride for the final step.

LC-MS: t_{R}=1.02 min; [M+H]⁺=680.22.

### Example 72

### (S)—N-[1-Benzyl-2-(8-methoxy-3,4-dihydro-1H-isoquinolin-2-yl)-2-oxo-ethyl]-N-(4-pyridin-2-yl-benzyl)-3-(4-trifluoromethyl-phenyl)-acrylamide

This compound has been prepared according to the methods of example 66 but using 8-methoxy-1,2,3,4-tetrahydro-isoquinoline hydrochloride for the final step.

LC-MS: t_{R}=1.00 min; [M+H]⁺=676.22.

### Example 73

### (S)—N-[1-Benzyl-2-(5-chloro-3,4-dihydro-1H-isoquinolin-2-yl)-2-oxo-ethyl]-N-(4-pyridin-2-yl-benzyl)-3-(4-trifluoromethyl-phenyl)-acrylamide

This compound has been prepared according to the methods of example 66 but using 5-chloro-1,2,3,4-tetrahydro-isoquinoline hydrochloride for the final step.

LC-MS: t_{R}=1.02 min; [M+H]⁺=680.20.

### Example 74

### (S)—N-[1-Benzyl-2-(5-fluoro-3,4-dihydro-1H-isoquinolin-2-yl)-2-oxo-ethyl]-N-(4-pyridin-2-yl-benzyl)-3-(4-trifluoromethyl-phenyl)-acrylamide

This compound has been prepared according to the methods of example 66 but using 5-fluoro-1,2,3,4-tetrahydro-isoquinoline hydrochloride for the final step.

LC-MS: t_{R}=1.01 min; [M+H]⁺=664.23.

### Example 75

### (S)—N-[1-Benzyl-2-(5-methoxy-3,4-dihydro-1H-isoquinolin-2-yl)-2-oxo-ethyl]-N-(4-pyridin-2-yl-benzyl)-3-(4-trifluoromethyl-phenyl)-acrylamide

This compound has been prepared according to the methods of example 66 but using 5-methoxy-1,2,3,4-tetrahydro-isoquinoline hydrochloride for the final step.

LC-MS: t_{R}=1.01 min; [M+H]⁺=676.28.

### Example 76

### (S)—N-[1-Benzyl-2-(6-methoxy-3,4-dihydro-1H-isoquinolin-2-yl)-2-oxo-ethyl]-N-(4-pyridin-2-yl-benzyl)-3-(4-trifluoromethyl-phenyl)-acrylamide

This compound has been prepared according to the methods of example 66 but using 6-methoxy-1,2,3,4-tetrahydro-isoquinoline hydrochloride for the final step.

LC-MS: t_{R}=1.00 min; [M+H]⁺=676.22.

### Example 77

### (S)—N-[1-Benzyl-2-(6-chloro-3,4-dihydro-1H-isoquinolin-2-yl)-2-oxo-ethyl]-N-(4-pyridin-2-yl-benzyl)-3-(4-trifluoromethyl-phenyl)-acrylamide

This compound has been prepared according to the methods of example 66 but using 6-chloro-1,2,3,4-tetrahydro-isoquinoline hydrochloride for the final step.

LC-MS: t_{R}=1.03 min; [M+H]⁺=680.39.

### Example 78

### (S)—N-[1-Benzyl-2-(6-fluoro-3,4-dihydro-1H-isoquinolin-2-yl)-2-oxo-ethyl]-N-(4-pyridin-2-yl-benzyl)-3-(6-trifluoromethyl-pyridin-3-yl)-acrylamide

This compound has been prepared according to the methods of example 66 but using 3-(6-trifluoromethyl-pyridin-3-yl)-acryloyl chloride for step 2 and 6-fluoro-1,2,3,4-tetrahydro-isoquinoline hydrochloride for the final step.

LC-MS: t_{R}=1.01 min; [M+H]⁺=665.27.

### Example 79

### rac-N-[2-(3,4-Dihydro-1H-isoquinolin-2-yl)-1-(1-methyl-1H-pyrazol-4-ylmethyl)-2-oxo-ethyl]-N-(4-pyridin-2-yl-benzyl)-3-(4-trifluoromethyl-phenyl)-acrylamide

This compound has been prepared according to the methods of example 66 but using 2-amino-3-(1-methyl-1H-pyrazol-4-yl)-propionic acid methyl ester for step 1 and 1,2,3,4-tetrahydro-isoquinoline for the final step.

LC-MS: t_{R}=0.98 min; [M+H]⁺=649.87.

### Example 80

### rac-N-[2-(3,4-Dihydro-1H-isoquinolin-2-yl)-1-(1-methyl-1H-pyrazol-4-ylmethyl)-2-oxo-ethyl]-N-(4-pyridin-4-yl-benzyl)-3-(4-trifluoromethyl-phenyl)-acrylamide

This compound has been prepared according to the methods of example 66 but using 2-amino-3-(1-methyl-1H-pyrazol-4-yl)-propionic acid methyl ester (prepared according to pathway D) and commercially available 4-pyridin-4-yl-benzaldehyde for step 1 and 1,2,3,4-tetrahydro-isoquinoline for the final step.

LC-MS: t_{R}=0.92 min; [M+H]⁺=649.80.

### Example 81

### rac-N-[2-(3,4-Dihydro-1H-isoquinolin-2-yl)-1-(1-methyl-1H-pyrazol-3-ylmethyl)-2-oxo-ethyl]-N-(4-pyridin-2-yl-benzyl)-3-(4-trifluoromethyl-phenyl)-acrylamide

This compound has been prepared according to the methods of example 66 but using 2-amino-3-(1-methyl-1H-pyrazol-3-yl)-propionic acid methyl ester (prepared according to pathway D) for step 1 and 1,2,3,4-tetrahydro-isoquinoline for the final step.

LC-MS: t_{R}=0.99 min; [M+H]⁺=649.86.

### Example 82

### rac-N-[2-(3,4-Dihydro-1H-isoquinolin-2-yl)-1-(1-methyl-1H-pyrazol-3-ylmethyl)-2-oxo-ethyl]-N-(4-pyridin-4-yl-benzyl)-3-(4-trifluoromethyl-phenyl)-acrylamide

This compound has been prepared according to the methods of example 66 but using 2-amino-3-(1-methyl-1H-pyrazol-3-yl)-propionic acid methyl ester (prepared according to pathway D) and commercially available 4-pyridin-4-yl-benzaldehyde for step 1 and 1,2,3,4-tetrahydro-isoquinoline for the final step.

LC-MS: t_{R}=0.94 min; [M+H]⁺=649.83.

### Example 83

### rac-N-[2-(3,4-Dihydro-1H-isoquinolin-2-yl)-1-(2-methyl-2H-pyrazol-3-ylmethyl)-2-oxo-ethyl]-N-(4-pyridin-2-yl-benzyl)-3-(4-trifluoromethyl-phenyl)-acrylamide

This compound has been prepared according to the methods of example 66 but using 2-amino-3-(2-methyl-2H-pyrazol-3-yl)-propionic acid methyl ester (prepared according to pathway D) for step 1 and 1,2,3,4-tetrahydro-isoquinoline for the final step.

LC-MS: t_{R}=0.99 min; [M+H]⁺=649.86.

### Example 84

### rac-N-[2-(3,4-Dihydro-1H-isoquinolin-2-yl)-1-(2-methyl-2H-pyrazol-3-ylmethyl)-2-oxo-ethyl]-N-(4-pyridin-4-yl-benzyl)-3-(4-trifluoromethyl-phenyl)-acrylamide

This compound has been prepared according to the methods of example 66 but using 2-amino-3-(2-methyl-2H-pyrazol-3-yl)-propionic acid methyl ester (prepared according to pathway D) and commercially available 4-pyridin-4-yl-benzaldehyde for step 1 and 1,2,3,4-tetrahydro-isoquinoline for the final step.

LC-MS: t_{R}=0.94 min; [M+H]⁺=649.88.

### Example 85

### rac-N-[2-(3,4-Dihydro-1H-isoquinolin-2-yl)-2-oxo-1-pyrimidin-2-ylmethyl-ethyl]-N-(4-pyridin-2-yl-benzyl)-3-(4-trifluoromethyl-phenyl)-acrylamide

This compound has been prepared according to the methods of example 66 but using 2-amino-3-pyrimidin-2-yl-propionic acid ethyl ester (prepared according to pathway E) for step 1 and 1,2,3,4-tetrahydro-isoquinoline for the final step.

LC-MS: t_{R}=0.98 min; [M+H]⁺=647.99.

### Example 86

### rac-N-[2-(3,4-Dihydro-1H-isoquinolin-2-yl)-2-oxo-1-pyrimidin-2-ylmethyl-ethyl]-N-(4-pyridin-4-yl-benzyl)-3-(4-trifluoromethyl-phenyl)-acrylamide

This compound has been prepared according to the methods of example 66 but using 2-amino-3-pyrimidin-2-yl-propionic acid ethyl ester (prepared according to pathway E) and commercially available 4-pyridin-4-yl-benzaldehyde for step 1 and 1,2,3,4-tetrahydro-isoquinoline for the final step.

LC-MS: t_{R}=0.93 min; [M+H]⁺=648.20.

### Example 87

### rac-N-[2-(3,4-Dihydro-1H-isoquinolin-2-yl)-1-isoxazol-3-ylmethyl-2-oxo-ethyl]-N-(4-pyridin-4-yl-benzyl)-3-(4-trifluoromethyl-phenyl)-acrylamide

This compound has been prepared according to the methods of example 66 but using 2-amino-3-isoxazol-3-yl-propionic acid ethyl ester (prepared according to pathway E) and commercially available 4-pyridin-4-yl-benzaldehyde for step 1 and 1,2,3,4-tetrahydro-isoquinoline for the final step.

LC-MS: t_{R}=0.96 min; [M+H]⁺=636.96.

In vitro antimalarial activity: Plasmodium falciparum in vitro assay In vitro activity against erythrocytic stages of P. falciparum is determined using a [³H] hypoxanthine incorporation assay. One strain resistant to chloroquine and pyrimethamine (P. falciparum K1) is used in the assays, and all test compounds are compared for activity with the standard drugs chloroquine (sigma C6628) and artemisinin (sigma-36, 159-3). Compounds are diluted in DMSO to 1 mM and added to parasite cultures incubated in RPMI 1640 medium without hypoxanthine, supplemented with HEPES (5.94 g/L), NaHCO₃ (2.1 g/L), neomycin (100 U/mL), Albumax (5 g/L) and washed human red cells at 2.5% hematocrit (0.3% parasitaemia). Seven serial doubling dilutions of each drug are prepared in 96-well microtitre plates and incubated in a humidifying atmosphere at 37° C.; 4% CO₂, 3% O₂, 93% N₂.

After 48 h, 50 μl of [³H] hypoxanthine (0.5 μCi) is added to each well of a plate. The plates are incubated for a further 24 h under the same conditions. The plates are then harvested with a Betaplate cell harvester (Wallac) and washed with distilled water. The dried filters are inserted into a plastic foil with 10 mL of scintillation fluid, and counted in a Betaplate liquid scintillation counter. IC₅₀ values are calculated from sigmoidal inhibition curves using Microsoft Excel. Inhibition activities (IC₅₀ values) of the 254 exemplified compounds are in the range of 1-423 nM with an average of 257 nM with respect to the Plasmodium Falciparum strain K1.

TABLE 1
IC₅₀ values (nM) for some compounds of formula I:

| Compound of Example No.: | IC₅₀ (nM) on K1 |
|---|---|
| Example 1 | 3.5 |
| Example 4 | 5 |
| Example 6 | 25 |
| Example 10 | 14 |
| Example 14 | 18 |
| Example 25 | 13 |
| Example 32 | 7.5 |
| Example 38 | 10 |
| Example 51 | 17 |
| Example 59 | 14 |
| Example 62 | 10 |
| Example 65 | 10 |
| Example 69 | 12 |
| Example 74 | 11 |
| Example 78 | 52 |
| Example 80 | 107 |
| Example 82 | 24 |
| Example 84 | 84 |
| Example 86 | 162.5 |
| Example 87 | 194 |
| Example 93 | 19 |
| Example 99 | 29 |
| Example 104 | 84 |
| Example 111 | 35 |
| Example 114 | 63 |
| Example 116 | 67 |
| Example 119 | 5 |
| Example 145 | 13 |
| Example 157 | 2 |
| Example 167 | 8 |
| Example 172 | 25 |
| Example 178 | 28 |
| Example 199 | 4 |
| Example 211 | 82 |
| Example 218 | 31 |
| Example 220 | 10 |
| Example 224 | 18 |
| Example 228 | 8.5 |
| Example 235 | 26 |
| Example 239 | 9 |
| Example 241 | 5 |
| Example 243 | <7.8 |
| Example 246 | 17 |
| Example 247 | 4 |
| Example 250 | 2 |
| Example 251 | 42 |
| Example 253 | 70 |
| Example 254 | <7.8 |
| Chloroquine | 300 |
| Artemisinin | 2 |

### In Vivo Antimalarial Efficacy Studies

In vivo antimalarial activity is assessed for groups of three female NMRI mice (20-22 g) intravenously infected on day 0 with P. berghei strain GFP-ANKA (0.2 mL heparinized saline suspension containing 2×10⁷ parasitized erythrocytes). In control mice, parasitaemia typically rise to approximately 40% by day 3 after infection, and control mice die between day 5 and day 7 after infection. For the mice treated with compounds, the compounds are either formulated in an aqueous-gelatine vehicle with 3 mg/mL compounds or in tween 80/ethanol (7%/3%) with 5 mg/mL.

Compounds are administered intraperitonealy or subcutaneously either as two consecutive twice-daily dosing (BID) (2×75 mg/kg BID, 24 and 48 hours after infection) or as four consecutive daily doses (4×10 mg/kg or 4×50 mg/kg, 3, 24, 48 and 72 hours after infection). With the double BID-dose regimen, 24 h after the last drug treatment, 1 μl tail blood is taken, resuspended in 1 mL PBS buffer and parasitemia determined with a FACScan (Becton Dickinson) by counting 100 000 red blood cells. Tail blood samples for the quadruple-dose regimen are processed on day 4 after infection. Activity is calculated as the difference between the mean value of the control and treated groups expressed as a percent relative to the control group. For parasetimias lower than 0.1%, the presence of parasites in the FACS gate is checked visually. The survival days of infected mice treated with compound is also recorded for each compound. Mice surviving for 30 days are checked for parasitemia and subsequently euthanized. A compound is considered curative if the animal survives to day 30 post-infection with no detectable parasites.

1. A compound of the formula I: 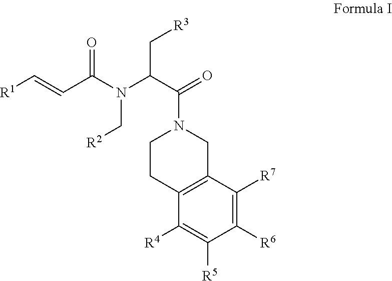
wherein
R¹ represents aryl or heteroaryl, wherein these two radicals can optionally be mono-, di-, tri-, or tetra-substituted, wherein the substituents are independently selected from the group consisting of halogen, (C₁-C₄)alkyl, (C₁-C₄)alkoxy, cycloalkyl, trifluoromethyl, trifluoromethoxy, and amino, wherein the amino group is optionally mono- or di-substituted with (C₁-C₄)alkyl or mono-substituted with (C₁-C₄)alkyl-carbonyl; or R¹ represents aryl wherein two adjacent carbon ring atoms of the aryl moiety are substituted with (C₁-C₂)alkylenedioxy, wherein the (C₁-C₂)alkylene moiety is optionally mono- or di-substituted, wherein the substituents are independently selected from the group consisting of halogen and (C₁-C₄)alkyl;
R² represents aryl or heteroaryl, wherein these two radicals can optionally be mono-, di-, tri-, or tetra-substituted, wherein the substituents are independently selected from the group consisting of halogen; (C₁-C₄)alkyl; (C₁-C₄)alkoxy; cycloalkyl; trifluoromethyl; trifluoromethoxy; heterocycloalkyl, that can optionally be mono-substituted on one nitrogen ring atom, if present, with (C₁-C₄)alkyl or (C₁-C₄)alkyl-carbonyl; aryl or heteroaryl, wherein these two radicals can optionally be mono-, di-, tri-, or tetra-substituted, wherein the substituents are independently selected from the group consisting of halogen, (C₁-C₄)alkyl, (C₁-C₄)alkoxy, cycloalkyl, trifluoromethyl, and trifluoromethoxy; 2-methyl-2H-tetrazol-5-yl; and 1-oxy-pyridin-4-yl;
R³ represents aryl or heteroaryl, wherein these two radicals can optionally be mono-, di-, tri-, or tetra-substituted, wherein the substituents are independently selected from the group consisting of halogen, (C₁-C₄)alkyl, (C₁-C₄)alkoxy, cycloalkyl, trifluoromethyl, and trifluoromethoxy; or R³ represents heterocycloalkyl that can optionally be mono-substituted on one nitrogen ring atom, if present, with (C₁-C₄)alkyl or (C₁-C₄)alkyl-carbonyl; and
R⁴, R⁵, R⁶, and R⁷ independently represent hydrogen, halogen, (C₁-C₄)alkyl, (C₁-C₄)alkoxy, cycloalkyl, or trifluoromethyl;
or salts thereof.

2. A compound according to claim 1, wherein the carbon atom to which —CH₂—R³ is attached is in the (S)-configuration: 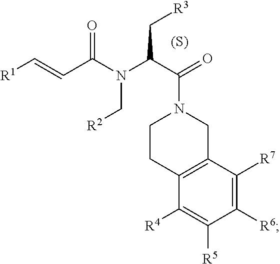
or a salt thereof.

3. A compound according to claim 1, wherein
R¹ represents mono-substituted aryl or mono-substituted heteroaryl, wherein the substituent is selected from the group consisting of (C₁-C₄)alkyl, (C₁-C₄)alkoxy, cycloalkyl, trifluoromethyl, and trifluoromethoxy;
or a salt thereof.

4. A compound according to claim 3, wherein R¹ represents mono-substituted aryl or mono-substituted heteroaryl, wherein the substituent is selected from the group consisting of methyl, methoxy, and trifluoromethyl;
or a salt thereof.

5. A compound according to claim 1, wherein
R¹ represents phenyl, pyridyl or pyrimidyl, wherein these three radicals are mono-, di-, or tri-substituted, wherein the substituents are independently selected from the group consisting of halogen, (C₁-C₄)alkyl, (C₁-C₄)alkoxy, trifluoromethyl, and amino, wherein the amino group is optionally mono- or di-substituted with (C₁-C₄)alkyl; or R¹ represents pyrazolyl, imidazolyl, thiazolyl, isoxazolyl, oxazolyl, thiadiazolyl, or pyridazinyl, wherein these radicals are mono-, di-, or tri-substituted, wherein the substituents are independently selected from the group consisting of (C₁-C₄)alkyl and (C₁-C₄)alkoxy; or R¹ represents phenyl, wherein two adjacent carbon ring atoms of the phenyl moiety are substituted with (C₁-C₂)alkylenedioxy;
or a salt thereof.

6. A compound according to claim 1, wherein
R² represents mono-substituted aryl or mono-substituted heteroaryl, wherein the substituent is selected from the group consisting of halogen, (C₁-C₄)alkyl, (C₁-C₄)alkoxy, cycloalkyl, trifluoromethyl, trifluoromethoxy, aryl, heteroaryl, and heterocycloalkyl wherein the heterocycloalkyl can optionally be mono-substituted on one nitrogen ring atom, if present, with (C₁-C₄)alkyl or (C₁-C₄)alkyl-carbonyl;
or a salt thereof.

7. A compound according to claim 1, wherein
R² represents phenyl or pyridyl, wherein these two radicals can optionally be mono-substituted with a substituent selected from the group consisting of (C₁-C₄)alkyl; morpholinyl; piperazinyl mono-substituted on one nitrogen ring atom with (C₁-C₄)alkyl; pyridyl; pyrimidyl; pyrazinyl; pyridazinyl; triazolyl; pyrazolyl; thiazolyl; oxazolyl; 2-methyl-2H-tetrazol-5-yl; and 1-oxy-pyridin-4-yl;
or a salt thereof.

8. A compound according to claim 1, wherein
R³ represents phenyl;
or a salt thereof.

9. A compound according to claim 1, wherein
R⁴, R⁵, R⁶, and R⁷ all represent hydrogen;
or a salt thereof.

10. A compound according to claim 1, wherein
R¹ represents phenyl, pyridyl or pyrimidyl, wherein these three radicals are mono-, di-, or tri-substituted, wherein the substituents are independently selected from the group consisting of halogen, (C₁-C₄)alkyl, (C₁-C₄)alkoxy, trifluoromethyl, and amino, wherein the amino group is optionally mono- or di-substituted with (C₁-C₄)alkyl; or R¹ represents 1-methyl-1H-pyrazol-3-yl, 2,5-dimethyl-2H-pyrazol-3-yl, 1,3,5-trimethyl-1H-pyrazol-4-yl, 2,3-dimethyl-3H-imidazol-4-yl, 2,4-dimethyl-thiazol-5-yl, 2-methyl-thiazol-4-yl, 5-methyl-isoxazol-3-yl, 3,5-dimethyl-isoxazol-4-yl, 2,5-dimethyl-oxazol-4-yl, [1,2,3]thiadiazol-4-yl, 1,5-dimethyl-1H-pyrazol-4-yl, 6-methoxy-pyridazin-3-yl, 1-methyl-1H-pyrazol-4-yl, 4-methyl-thiazol-5-yl, or 2-methyl-thiazol-5-yl; or R¹ represents phenyl, wherein two adjacent carbon ring atoms of the phenyl moiety are substituted with (C₁-C₂)alkylenedioxy;
R² represents phenyl or pyridyl, wherein these two radicals can optionally be mono-substituted with a substituent selected from the group consisting of (C₁-C₄)alkyl, morpholinyl, 4-methyl-piperazin-1-yl, pyridyl, pyrimidyl, pyrazin-2-yl, pyridazin-3-yl, [1,2,3]triazol-1-yl, [1,2,4]triazol-1-yl, pyrazol-1-yl, thiazol-2-yl, thiazol-5-yl, oxazol-5-yl, 2-methyl-2H-tetrazol-5-yl, and 1-oxy-pyridin-4-yl;
R³ represents phenyl, pyridyl, pyrimidyl, isoxazolyl, or methyl-pyrazolyl; and
R⁴, R⁵, R⁶, and R⁷ independently represent hydrogen, halogen, (C₁-C₄)alkoxy, or trifluoromethyl;
or a salt thereof.

11. A compound according to claim 1, selected from the group consisting of:
(S)—N-[1-Benzyl-2-(3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-N-(4-pyridin-2-yl-benzyl)-3-(6-trifluoromethyl-pyridin-3-yl)-acrylamide;
(S)—N-[1-Benzyl-2-(3,4-dihydro-1H-isoquinolin-2-yl)-2-oxo-ethyl]-N-(4-pyrimidin-5-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-N-(4-pyrimidin-5-yl-benzyl)-3-(6-trifluoromethyl-pyridin-3-yl)-acrylamide;
(S)—N-[1-Benzyl-2-(3,4-dihydro-1H-isoquinolin-2-yl)-2-oxo-ethyl]-N-(6-morpholin-4-yl-pyridin-3-ylmethyl)-3-(4-trifluoromethyl-phenyl)-acrylamide;
(S)—N-[1-Benzyl-2-(3,4-dihydro-1H-isoquinolin-2-yl)-2-oxo-ethyl]-N-pyridin-2-ylmethyl-3-(4-trifluoromethyl-phenyl)-acrylamide;
(S)—N-Benzyl-N-[1-benzyl-2-(3,4-dihydro-1H-isoquinolin-2-yl)-2-oxo-ethyl]-3-(4-trifluoromethyl-phenyl)-acrylamide;
(S)—N-[1-Benzyl-2-(3,4-dihydro-1H-isoquinolin-2-yl)-2-oxo-ethyl]-3-(4-methoxy-phenyl)-N-(6-morpholin-4-yl-pyridin-3-ylmethyl)-acrylamide;
(S)—N-[1-Benzyl-2-(6,7-ethoxy-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(6-fluoro-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(3,4-dihydro-1-H-isoquinolin-2-yl)-2-oxo-ethyl]-3-(4-methoxy-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-N-(4-pyridin-2-yl-benzyl)-3-(3-trifluoromethyl-phenyl)-acrylamide;
(S)—N-[1-Benzyl-2-(3,4-dihydro-1-H-isoquinolin-2-yl)-2-oxo-ethyl]-3-(4-bromo-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(3,5-dimethoxy-phenyl)-N-(4-pyridin-2-yl-benzyl)-acrylamide;
(S)—N-Benzyl-N-[1-benzyl-2-(3,4-dihydro-1H-isoquinolin-2-yl)-2-oxo-ethyl]-3-(6-trifluoromethyl-pyridin-3-O-acrylamide;
(S)—N-[1-Benzyl-2-(6-fluoro-3,4-dihydro-1H-isoquinolin-2-yl)-2-oxo-ethyl]-N-(4-pyridin-2-yl-benzyl)-3-(6-trifluoromethyl-pyridin-3-yl)-acrylamide;
(S)—N-[1-Benzyl-2-(3,4-dihydro-1H-isoquinolin-2-yl)-2-oxo-ethyl]-N-(6-morpholin-4-yl-pyridin-3-ylmethyl)-3-p-tolyl-acrylamide;
(S)—N-[1-Benzyl-2-(3,4-dihydro-1-H-isoquinolin-2-yl)-2-oxo-ethyl]-3-(3-methoxy-phenyl)-N-(4-pyridin-2-yl-benzyl)-acrylamide;
(S)-3-Benzo[1,3]dioxol-5-yl-N-[benzyl-2-(3,4-dihydro-1H-isoquinolin-2-yl)-2-oxo-ethyl]-N-(4-pyridin-2-yl-benzyl)-acrylamide;
(S)—N-[1-Benzyl-2-(3,4-dihydro-1-H-isoquinolin-2-yl)-2-oxo-ethyl]-3-(4-fluoro-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(2,4-difluoro-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(2-bromo-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(3,4-difluoro-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(3-bromo-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(2,5-dimethyl-phenyl)-N-(4-pyridin-2-yl-benzyl)-acrylamide;
(S)—N-Benzyl-N-[1-Benzyl-2-(3,4-dihydro-1-H-isoquinolin-2-yl)-2-oxo-ethyl]-3-p-tolyl-acrylamide;
(S)—N-[1-Benzyl-2-(3,4-dihydro-1H-isoquinolin-2-yl)-2-oxo-ethyl]-N-(4-ethyl-benzyl)-3-p-tolyl-acrylamide;
(S)—N-[1-Benzyl-2-(3,4-dihydro-1-H-isoquinolin-2-yl)-2-oxo-ethyl]-3-(2,3-dichloro-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(2-chloro-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(3-fluoro-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(2,5-dimethoxy-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(3,4,5-trimethoxy-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-N-(4-pyridin-2-yl-benzyl)-3-o-tolyl-acrylamide;
(S)—N-[1-Benzyl-2-(3,4-dihydro-1-H-isoquinolin-2-yl)-2-oxo-ethyl]-N-(4-pyridin-2-yl-benzyl)-3-m-tolyl-acrylamide;
(S)—N-[1-Benzyl-2-(3,4-dihydro-1-H-isoquinolin-2-yl)-2-oxo-ethyl]-N-(4-pyridin-2-yl-benzyl)-3-p-tolyl-acrylamide;
(S)—N-[1-Benzyl-2-(3,4-dihydro-1-H-isoquinolin-2-yl)-2-oxo-ethyl]-3-(2,4-dichloro-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(2-methoxy-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(4-chloro-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(3-chloro-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-3-(4-dimethylamino-phenyl)-N-(4-pyridin-2-yl-benzyl)-acrylamide;
(S)—N-[1-Benzyl-2-(3,4-dihydro-1-H-isoquinolin-2-yl)-2-oxo-ethyl]-N-(4-pyridin-4-yl-benzyl)-3-(4-trifluoromethyl-phenyl)-acrylamide;
(S)—N-[2-(3,4-Dihydro-1H-isoquinolin-2-yl)-2-oxo-1-pyridin-2-ylmethyl-ethyl]-N-(4-ethyl-benzyl)-3-p-tolyl-acrylamide;
(S)—N-[1-Benzyl-2-(3,4-dihydro-1H-isoquinolin-2-yl)-2-oxo-ethyl]-N-pyridin-3-ylmethyl-3-p-tolyl-acrylamide;
(S)—N-[1-Benzyl-2-(3,4-dihydro-1H-isoquinolin-2-yl)-2-oxo-ethyl]-3-(2,3,6-trifluoro-phenyl)-N-(4-pyridin-2-yl-benzyl)-acrylamide;
(S)—N-[1-Benzyl-2-(3,4-dihydro-1H-isoquinolin-2-yl)-2-oxo-ethyl]-3-(2,5-difluoro-phenyl)-N-(4-pyridin-2-yl-benzyl)-acrylamide;
(S)—N-[1-Benzyl-2-(3,4-dihydro-1H-isoquinolin-2-yl)-2-oxo-ethyl]-3-(2-trifluoromethyl-phenyl)-N-(4-pyridin-2-yl-benzyl)-acrylamide;
(S)—N-[1-Benzyl-2-(6-fluoro-3,4-dihydro-1H-isoquinolin-2-yl)-2-oxo-ethyl]-N-(4-pyridin-2-yl-benzyl)-3-(6-trifluoromethyl-pyridin-3-yl)-acrylamide;
(S)—N-Benzyl-N-[1-benzyl-2-(3,4-dihydro-1H-isoquinolin-2-yl)-2-oxo-ethyl]-3-(6-trifluoromethyl-pyridin-3-yl)-acrylamide;
(S)—N-[1-Benzyl-2-(3,4-dihydro-1H-isoquinolin-2-yl)-2-oxo-ethyl]-N-(6-morpholin-4-yl-pyridin-3-ylmethyl)-3-p-tolyl-acrylamide;
(S)—N-[1-Benzyl-2-oxo-2-(6-trifluoromethyl-3,4-dihydro-1H-isoquinolin-2-yl)-ethyl]-N-(4-pyridin-2-yl-benzyl)-3-(4-trifluoromethyl-phenyl)-acrylamide;
(S)—N-[1-Benzyl-2-oxo-2-(7-trifluoromethyl-3,4-dihydro-1H-isoquinolin-2-yl)-ethyl]-N-(4-pyridin-2-yl-benzyl)-3-(4-trifluoromethyl-phenyl)-acrylamide;
(S)—N-[1-Benzyl-2-(7-chloro-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(7-methoxy-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(7-fluoro-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(8-chloro-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(8-methoxy-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(5-chloro-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(5-fluoro-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(5-methoxy-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(6-methoxy-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(6-chloro-3,4-dihydro-1H-isoquinolin-2-yl)-2-oxo-ethyl]-N-(4-pyridin-2-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-3-(1-methyl-1H-pyrazol-3-yl)-N-(4-pyridin-2-yl-benzyl)-acrylamide;
(S)—N-[1-Benzyl-2-(3,4-dihydro-1H-isoquinolin-2-yl)-2-oxo-ethyl]-3-(2,5-dimethyl-2H-pyrazol-3-yl)-N-(4-pyridin-2-yl-benzyl)-acrylamide;
(S)—N-[1-Benzyl-2-(3,4-dihydro-1H-isoquinolin-2-yl)-2-oxo-ethyl]-N-(4-pyridin-2-yl-benzyl)-3-(1,3,5-trimethyl-1H-pyrazol-4-yl)-acrylamide;
(S)—N-[1-Benzyl-2-(3,4-dihydro-1H-isoquinolin-2-yl)-2-oxo-ethyl]-3-(2,3-dimethyl-3H-imidazol-4-yl)-N-(4-pyridin-2-yl-benzyl)-acrylamide;
(S)—N-[1-Benzyl-2-(3,4-dihydro-1H-isoquinolin-2-yl)-2-oxo-ethyl]-3-(2,4-dimethyl-thiazol-5-yl)-N-(4-pyridin-2-yl-benzyl)-acrylamide;
(S)—N-[1-Benzyl-2-(3,4-dihydro-1H-isoquinolin-2-yl)-2-oxo-ethyl]-3-(2-methyl-thiazol-4-yl)-N-(4-pyridin-2-yl-benzyl)-acrylamide;
(S)—N-[1-Benzyl-2-(3,4-dihydro-1H-isoquinolin-2-yl)-2-oxo-ethyl]-3-(5-methyl-isoxazol-3-yl)-N-(4-pyridin-2-yl-benzyl)-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-(4-pyridin-2-yl-benzyl)-acrylamide;
(S)—N-[1-Benzyl-2-(3,4-dihydro-1H-isoquinolin-2-yl)-2-oxo-ethyl]-3-(2,5-dimethyl-oxazol-4-yl)-N-(4-pyridin-2-yl-benzyl)-acrylamide;
(S)—N-[1-Benzyl-2-(3,4-dihydro-1H-isoquinolin-2-yl)-2-oxo-ethyl]-N-(4-pyridin-2-yl-benzyl)-3-[1,2,3]thiadiazol-4-yl-acrylamide;
(S)—N-[1-Benzyl-2-(3,4-dihydro-1H-isoquinolin-2-yl)-2-oxo-ethyl]-N-(4-pyridin-2-yl-benzyl)-3-(5-trifluoromethyl-pyridin-2-yl)-acrylamide;
(S)—N-[1-Benzyl-2-(3,4-dihydro-1H-isoquinolin-2-yl)-2-oxo-ethyl]-3-(2-methyl-pyrimidin-5-yl)-N-(4-pyridin-2-yl-benzyl)-acrylamide;
(S)—N-[1-Benzyl-2-(3,4-dihydro-1H-isoquinolin-2-yl)-2-oxo-ethyl]-3-(6-methyl-pyridin-3-yl)-N-(4-pyridin-2-yl-benzyl)-acrylamide;
(S)—N-[1-Benzyl-2-(3,4-dihydro-1H-isoquinolin-2-yl)-2-oxo-ethyl]-3-(6-chloro-pyridin-3-yl)-N-(4-pyridin-2-yl-benzyl)-acrylamide;
(S)—N-[1-Benzyl-2-(3,4-dihydro-1H-isoquinolin-2-yl)-2-oxo-ethyl]-3-(5-methyl-pyridin-2-yl)-N-(4-pyridin-2-yl-benzyl)-acrylamide;
(S)—N-[1-Benzyl-2-(3,4-dihydro-1H-isoquinolin-2-yl)-2-oxo-ethyl]-3-(6-methoxy-pyridin-3-yl)-N-(4-pyridin-2-yl-benzyl)-acrylamide;
(S)—N-[1-Benzyl-2-(3,4-dihydro-1H-isoquinolin-2-yl)-2-oxo-ethyl]-3-(5-methoxy-pyridin-2-yl)-N-(4-pyridin-2-yl-benzyl)-acrylamide;
(S)—N-[1-Benzyl-2-(3,4-dihydro-1H-isoquinolin-2-yl)-2-oxo-ethyl]-N-(4-pyridin-2-yl-benzyl)-3-(2-trifluoromethyl-pyrimidin-5-yl)acrylamide; and
(S)—N-[1-Benzyl-2-(3,4-dihydro-1H-isoquinolin-2-yl)-2-oxo-ethyl]-3-(1,5-dimethyl-1H-pyrazol-4-yl)-N-(4-pyridin-2-yl-benzyl)-acrylamide;
or a salt thereof.

12. A compound according to claim 1, selected from the group consisting of:
(S)—N-[1-Benzyl-2-(3,4-dihydro-1-H-isoquinolin-2-yl)-2-oxo-ethyl]-3-(2-fluoro-phenyl)-N-(4-pyridin-2-yl-benzyl)-acrylamide;
(S)—N-[1-Benzyl-2-(3,4-dihydro-1H-isoquinolin-2-yl)-2-oxo-ethyl]-N-(4-pyrimidin-2-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-3-(5-methyl-pyridin-2-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(6-methyl-pyridin-3-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(6-chloro-pyridin-3-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(6-methoxy-pyridin-3-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(2-methyl-pyrimidin-5-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(6-methoxy-pyridazin-3-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(2-methoxy-pyrimidin-5-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(1-methyl-1H-pyrazol-4-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(2,5-dimethyl-2H-pyrazol-3-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(1,5-dimethyl-1H-pyrazol-4-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-N-(4-pyrimidin-2-yl-benzyl)-3-(1,3,5-trimethyl-1H-pyrazol-4-yl)-acrylamide;
(S)—N-[1-Benzyl-2-(3,4-dihydro-1H-isoquinolin-2-yl)-2-oxo-ethyl]-3-(2,3-dimethyl-3H-imidazol-4-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(2,5-dimethyl-oxazol-4-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(4-methyl-thiazol-5-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(2-methyl-thiazol-4-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(2-methyl-thiazol-5-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-3-(2,4-dimethyl-thiazol-5-yl)-N-(4-pyrimidin-2-yl-benzyl)-acrylamide;
(S)—N-[1-Benzyl-2-(3,4-dihydro-1H-isoquinolin-2-yl)-2-oxo-ethyl]-N-(4-pyrazin-2-benzyl)-3-(4-trifluoromethyl-phenyl)-acrylamide;
(S)—N-[1-Benzyl-2-(3,4-dihydro-1H-isoquinolin-2-yl)-2-oxo-ethyl]-3-(5-methyl-pyridin-2-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(6-methyl-pyridin-3-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(6-chloro-pyridin-3-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(6-methoxy-pyridin-3-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(2-methyl-pyrimidin-5-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(2-methoxy-pyrimidin-5-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(6-methoxy-pyridazin-3-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(1-methyl-1H-pyrazol-4-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(2,5-dimethyl-2H-pyrazol-3-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(1,5-dimethyl-1H-pyrazol-4-yl)-N-(4-pyrazin-2-yl-benzyl)-acrylamide;
(S)—N-[1-Benzyl-2-(3,4-dihydro-1H-isoquinolin-2-yl)-2-oxo-ethyl]-(4-pyrazin-2-yl-benzyl)-3-(1,3,5-trimethyl-1H-pyrazol-4-yl)-acrylamide;
(S)—N-[1-Benzyl-2-(3,4-dihydro-1H-isoquinolin-2-yl)-2-oxo-ethyl]-3-(2,3-dimethyl-3H-imidazol-4-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(5-methyl-isoxazol-3-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(2,5-dimethyl-oxazol-4-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(4-methyl-thiazol-5-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(2-methyl-thiazol-4-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(2-methyl-thiazol-5-yl)-N-(4-pyrazin-2-yl-benzyl)-acrylamide;
(S)—N-[1-Benzyl-2-(3,4-dihydro-1H-isoquinolin-2-yl)-2-oxo-ethyl]-3-(2,4-dimethyl-thiazol-5-yl)-N-(4-pyrazin-2-yl-benzyl)-acrylamide;
(S)—N-[1-Benzyl-2-(3,4-dihydro-1H-isoquinolin-2-yl)-2-oxo-ethyl]-N-(4-pyridazin-3-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-3-(5-methyl-pyridin-2-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(6-methyl-pyridin-3-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(6-chloro-pyridin-3-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(6-methoxy-pyridin-3-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(2-methyl-pyrimidin-5-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(2-methoxy-pyrimidin-5-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(6-methoxy-pyridazin-3-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(1-methyl-1H-pyrazol-4-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(2,5-dimethyl-2H-pyrazol-3-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(2,3-dimethyl-3H-imidazol-4-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(5-methyl-isoxazol-3-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(2,5-dimethyl-oxazol-4-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(4-methyl-thiazol-5-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(2-methyl-thiazol-4-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(2-methyl-thiazol-5-yl)-N-(4-pyridazin-3-yl-benzyl)-acrylamide;
(S)—N-[1-Benzyl-2-(3,4-dihydro-1H-isoquinolin-2-yl)-2-oxo-ethyl]-3-(2,4-dimethyl-thiazol-5-yl)-N-(4-pyridazin-3-yl-benzyl)-acrylamide;
(S)—N-[1-Benzyl-2-(3,4-dihydro-1H-isoquinolin-2-yl)-2-oxo-ethyl]-N-(4-[1,2,3]triazol-1-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-3-(5-methyl-pyridin-2-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(6-methyl-pyridin-3-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(6-chloro-pyridin-3-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(6-methoxy-pyridin-3-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(2-methyl-pyrimidin-5-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(2-methoxy-pyrimidin-5-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(6-methoxy-pyridazin-3-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(1-methyl-1H-pyrazol-4-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(2,5-dimethyl-2H-pyrazol-3-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(1,5-dimethyl-1H-pyrazol-4-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-N-(4-[1,2,3]-triazol-1-yl-benzyl)-3-(1,3,5-trimethyl-1H-pyrazol-4-yl)-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(2,5-dimethyl-oxazol-4-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(4-methyl-thiazol-5-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(2-methyl-thiazol-4-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(2-methyl-thiazol-5-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-3-(2,4-dimethyl-thiazol-5-yl)-N-(4-[1,2,3]-triazol-1-yl-benzyl)-acrylamide;
(S)—N-[1-Benzyl-2-(3,4-dihydro-1H-isoquinolin-2-yl)-2-oxo-ethyl]-N-(4-[1,2,4]triazol-1-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-3-(5-methyl-pyridin-2-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(6-methyl-pyridin-3-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(6-chloro-pyridin-3-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(6-methoxy-pyridin-3-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(2-methyl-pyrimidin-5-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(2-methoxy-pyrimidin-5-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(6-methoxy-pyridazin-3-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(1-methyl-1H-pyrazol-4-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(2,5-dimethyl-2H-pyrazol-3-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(1,5-dimethyl-1H-pyrazol-4-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-N-(4-[1,2,4]-triazol-1-yl-benzyl)-3-(1,3,5-trimethyl-1H-pyrazol-4-yl)-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(2,5-dimethyl-oxazol-4-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(4-methyl-thiazol-5-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(2-methyl-thiazol-4-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(2-methyl-thiazol-5-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-3-(2,4-dimethyl-thiazol-5-yl)-N-(4-[1,2,4]-triazol-1-yl-benzyl)-acrylamide;
(S)—N-[1-Benzyl-2-(3,4-dihydro-1H-isoquinolin-2-yl)-2-oxo-ethyl]-N-(4-pyrazol-1-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-3-(5-methyl-pyridin-2-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(6-methyl-pyridin-3-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(6-chloro-pyridin-3-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(6-methoxy-pyridin-3-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(2-methyl-pyrimidin-5-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(2-methoxy-pyrimidin-5-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(6-methoxy-pyridazin-3-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(1-methyl-1H-pyrazol-4-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(2,5-dimethyl-2H-pyrazol-3-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(1,5-dimethyl-1H-pyrazol-4-yl)-N-(4-pyrazol-1-yl-benzyl)-acrylamide;
(S)—N-[1-Benzyl-2-(3,4-dihydro-1H-isoquinolin-2-yl)-2-oxo-ethyl]-N-(4-pyrazol-1-yl-benzyl)-3-(1,3,5-trimethyl-1H-pyrazol-4-yl)-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(2,5-dimethyl-oxazol-4-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(4-methyl-thiazol-5-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(2-methyl-thiazol-5-yl)-N-(4-pyrazol-1-yl-benzyl)-acrylamide;
(S)—N-[1-Benzyl-2-(3,4-dihydro-1H-isoquinolin-2-yl)-2-oxo-ethyl]-3-(2,4-dimethyl-thiazol-5-yl)-N-(4-pyrazol-1-yl-benzyl)-acrylamide;
(S)—N-[1-Benzyl-2-(3,4-dihydro-1H-isoquinolin-2-yl)-2-oxo-ethyl]-N-(4-thiazol-2-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-N-(4-thiazol-2-yl-benzyl)-3-(1,3,5-trimethyl-1H-pyrazol-4-yl)-acrylamide;
(S)—N-[1-Benzyl-2-(3,4-dihydro-1H-isoquinolin-2-yl)-2-oxo-ethyl]-N-(4-thiazol-5-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-N-(4-thiazol-5-yl-benzyl)-3-(1,3,5-trimethyl-1H-pyrazol-4-yl)-acrylamide;
(S)—N-[1-Benzyl-2-(3,4-dihydro-1H-isoquinolin-2-yl)-2-oxo-ethyl]-N-(4-oxazol-5-yl-benzyl)-3-(4-trifluoromethyl-phenyl)-acrylamide;
(S)—N-[1-Benzyl-2-(3,4-dihydro-1H-isoquinolin-2-yl)-2-oxo-ethyl]-N-(4-oxazol-5-yl-benzyl)-3-(1,3,5-trimethyl-1H-pyrazol-4-yl)-acrylamide;
(S)—N-[1-Benzyl-2-(3,4-dihydro-1H-isoquinolin-2-yl)-2-oxo-ethyl]-N-[4-(2-methyl-2H-tetrazol-5-yl)-benzyl]-3-(4-trifluoromethyl-phenyl)-acrylamide;
(S)—N-[1-Benzyl-2-(3,4-dihydro-1H-isoquinolin-2-yl)-2-oxo-ethyl]-N-[4-(2-methyl-2H-tetrazol-5-yl)-benzyl]-3-(1,3,5-trimethyl-1H-pyrazol-4-yl)-acrylamide;
(S)—N-[2-(3,4-Dihydro-1H-isoquinolin-2-yl)-2-oxo-1-pyridin-2-ylmethyl-ethyl]-3-(4-methoxy-phenyl)-N-(6-morpholin-4-yl-pyridin-3-ylmethyl)-acrylamide;
(S)—N-[1-Benzyl-2-(3,4-dihydro-1H-isoquinolin-2-yl)-2-oxo-ethyl]-N-[4-(4-methyl-piperazin-1-yl)-benzyl]-3-(2-methyl-thiazol-4-yl)-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-[4-(4-methyl-piperazin-1-yl)-benzyl]acrylamide;
(S)—N-[1-Benzyl-2-(3,4-dihydro-1H-isoquinolin-2-yl)-2-oxo-ethyl]-3-(5-chloro-pyridin-2-yl)-N-[4-(4-methyl-piperazin-1-yl)-benzyl]-acrylamide;
(S)—N-[1-Benzyl-2-(3,4-dihydro-1H-isoquinolin-2-yl)-2-oxo-ethyl]-N-[4-(4-methyl-piperazin-1-yl)-benzyl]-3-(5-trifluoromethyl-pyridin-2-yl)-acrylamide;
(S)—N-[1-Benzyl-2-(3,4-dihydro-1H-isoquinolin-2-yl)-2-oxo-ethyl]-3-(6-methoxy-pyridin-3-yl)-N-[4-(4-methyl-piperazin-1-yl)-benzyl]-acrylamide;
(S)—N-[1-Benzyl-2-(3,4-dihydro-1H-isoquinolin-2-yl)-2-oxo-ethyl]-N-[4-(4-methyl-piperazin-1-yl)-benzyl]-3-(1,3,5-trimethyl-1H-pyrazol-4-yl)-acrylamide;
(S)—N-[1-Benzyl-2-(3,4-dihydro-1H-isoquinolin-2-yl)-2-oxo-ethyl]-3-(1,5-dimethyl-1H-pyrazol-4-yl)-N-[4-(4-methyl-piperazin-1-yl)-benzyl]-acrylamide;
(S)—N-[1-Benzyl-2-(3,4-dihydro-1H-isoquinolin-2-yl)-2-oxo-ethyl]-3-(1-methyl-1H-pyrazol-3-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(5-methyl-isoxazol-3-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(5-methyl-pyridin-2-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(6-methyl-pyridin-3-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(6-chloro-pyridin-3-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(6-methoxy-pyridin-3-yl)-N-(4-morpholin-4-yl-benzyl)-acrylamide;
(S)—N-[1-Benzyl-2-(3,4-dihydro-1H-isoquinolin-2-yl)-2-oxo-ethyl]-N-(4-morpholin-4-yl-benzyl)-3-(5-trifluoromethyl-pyridin-2-yl)-acrylamide;
(S)—N-[1-Benzyl-2-(3,4-dihydro-1H-isoquinolin-2-yl)-2-oxo-ethyl]-3-(2-methoxy-pyrimidin-5-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(2,5-dimethyl-2H-pyrazol-3-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(2,5-dimethyl-oxazol-4-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(2,3-dimethyl-3H-imidazol-4-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(5-methyl-pyridin-2-yl)-N-(4-pyrimidin-5-yl-benzyl)-acrylamide;
(S)—N-[1-Benzyl-2-(3,4-dihydro-1H-isoquinolin-2-yl)-2-oxo-ethyl]-3-(6-chloro-pyridin-3-yl)-N-(4-pyrimidin-5-yl-benzyl)-acrylamide;
(S)—N-[1-Benzyl-2-(3,4-dihydro-1H-isoquinolin-2-yl)-2-oxo-ethyl]-3-(6-methoxy-pyridin-3-yl)-N-(4-pyrimidin-5-yl-benzyl)-acrylamide;
(S)—N-[1-Benzyl-2-(3,4-dihydro-1H-isoquinolin-2-yl)-2-oxo-ethyl]-N-(4-pyrimidin-5-yl-benzyl)-3-(5-trifluoromethyl-pyridin-2-yl)-acrylamide;
(S)—N-[1-Benzyl-2-(3,4-dihydro-1H-isoquinolin-2-yl)-2-oxo-ethyl]-3-(2-methyl-pyrimidin-5-yl)-N-(4-pyrimidin-5-yl-benzyl)-acrylamide;
(S)—N-[1-Benzyl-2-(3,4-dihydro-1H-isoquinolin-2-yl)-2-oxo-ethyl]-3-(2,4-dimethyl-thiazol-5-yl)-N-(4-pyrimidin-5-yl-benzyl)-acrylamide;
(S)—N-[1-Benzyl-2-(3,4-dihydro-1H-isoquinolin-2-yl)-2-oxo-ethyl]-3-(2-methyl-thiazol-4-yl)-N-(4-pyrimidin-5-yl-benzyl)-acrylamide;
(S)—N-[1-Benzyl-2-(3,4-dihydro-1H-isoquinolin-2-yl)-2-oxo-ethyl]-3-(5-methyl-isoxazol-3-yl)-N-(4-pyrimidin-5-yl-benzyl)-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-(4-pyrimidin-5-yl-benzyl)-acrylamide;
(S)—N-[1-Benzyl-2-(3,4-dihydro-1H-isoquinolin-2-yl)-2-oxo-ethyl]-3-(5-methoxy-pyridin-2-yl)-N-(4-pyridin-4-yl-benzyl)-acrylamide;
(S)—N-[1-Benzyl-2-(3,4-dihydro-1H-isoquinolin-2-yl)-2-oxo-ethyl]-3-(5-methyl-pyridin-2-yl)-N-(4-pyridin-4-yl-benzyl)-acrylamide;
(S)—N-[1-Benzyl-2-(3,4-dihydro-1H-isoquinolin-2-yl)-2-oxo-ethyl]-3-(2-methyl-pyrimidin-5-yl)-N-(4-pyridin-4-yl-benzyl)-acrylamide;
(S)—N-[1-Benzyl-2-(3,4-dihydro-1H-isoquinolin-2-yl)-2-oxo-ethyl]-3-(6-methoxy-pyridazin-3-yl)-N-(4-pyridin-4-yl-benzyl)-acrylamide;
(S)—N-[1-Benzyl-2-(3,4-dihydro-1H-isoquinolin-2-yl)-2-oxo-ethyl]-N-(4-pyridin-4-yl-benzyl)-3-[1,2,3]thiadiazol-4-yl-acrylamide;
(S)—N-[1-Benzyl-2-(3,4-dihydro-1H-isoquinolin-2-yl)-2-oxo-ethyl]-3-(3,5-dimethyl-isoxazol-4-yl)-N-(4-pyridin-4-yl-benzyl)-acrylamide;
(S)—N-[1-Benzyl-2-(3,4-dihydro-1H-isoquinolin-2-yl)-2-oxo-ethyl]-3-(2,5-dimethyl-oxazol-4-yl)-N-(4-pyridin-4-yl-benzyl)-acrylamide;
(S)—N-[1-Benzyl-2-(3,4-dihydro-1H-isoquinolin-2-yl)-2-oxo-ethyl]-3-(1-methyl-1H-pyrazol-3-yl)-N-(4-pyridin-4-yl-benzyl)-acrylamide;
(S)—N-[1-Benzyl-2-(3,4-dihydro-1H-isoquinolin-2-yl)-2-oxo-ethyl]-3-(2,5-dimethyl-2H-pyrazol-3-yl)-N-(4-pyridin-4-yl-benzyl)-acrylamide;
(S)—N-[1-Benzyl-2-(3,4-dihydro-1H-isoquinolin-2-yl)-2-oxo-ethyl]-3-(1,3,5-trimethyl-1H-pyrazol-4-yl)-N-(4-pyridin-4-yl-benzyl)-acrylamide;
(S)—N-[1-Benzyl-2-(3,4-dihydro-1H-isoquinolin-2-yl)-2-oxo-ethyl]-3-(2,3-dimethyl-3H-imidazol-4-yl)-N-(4-pyridin-4-yl-benzyl)-acrylamide;
(S)—N-[1-Benzyl-2-(3,4-dihydro-1H-isoquinolin-2-yl)-2-oxo-ethyl]-3-(2,4-dimethyl-thiazol-5-yl)-N-(4-pyridin-4-yl-benzyl)-acrylamide;
(S)—N-[1-Benzyl-2-(3,4-dihydro-1H-isoquinolin-2-yl)-2-oxo-ethyl]-3-(2-methyl-thiazol-4-yl)-N-(4-pyridin-4-yl-benzyl)-acrylamide;
(S)—N-[1-Benzyl-2-(3,4-dihydro-1H-isoquinolin-2-yl)-2-oxo-ethyl]-3-(1,5-dimethyl-1H-pyrazol-4-yl)-N-(4-pyridin-4-yl-benzyl)-acrylamide;
(S)—N-[1-Benzyl-2-(3,4-dihydro-1H-isoquinolin-2-yl)-2-oxo-ethyl]-N-(4-pyridin-4-yl-benzyl)-3-(5-trifluoromethyl-pyridin-2-yl)-acrylamide;
(S)—N-[1-Benzyl-2-(3,4-dihydro-1H-isoquinolin-2-yl)-2-oxo-ethyl]-3-(2,4-dimethyl-thiazol-5-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(2-methyl-thiazol-4-yl)-N-(4-morpholin-4-yl-benzyl)-acrylamide;
(S)—N-[1-Benzyl-2-(3,4-dihydro-1H-isoquinolin-2-yl)-2-oxo-ethyl]-3-(2,5-dimethyl-oxazol-4-yl)-N-(4-pyrimidin-5-yl-benzyl)-acrylamide;
(S)—N-[1-Benzyl-2-(3,4-dihydro-1-H-isoquinolin-2-yl)-2-oxo-ethyl]-N-[4-(1-oxy-pyridin-4-yl)-benzyl]-3-(4-trifluoromethyl-phenyl)-acrylamide;
N-[2-(3,4-Dihydro-1H-isoquinolin-2-yl)-1-(1-methyl-1H-pyrazol-4-ylmethyl)-2-oxo-ethyl]-N-(4-pyridin-2-yl-benzyl)-3-(4-trifluoromethyl-phenyl)-acrylamide;
N-[2-(3,4-Dihydro-1H-isoquinolin-2-yl)-1-(1-methyl-1H-pyrazol-4-ylmethyl)-2-oxo-ethyl]-N-(4-pyridin-4-yl-benzyl)-3-(4-trifluoromethyl-phenyl)-acrylamide;
N-[2-(3,4-Dihydro-1H-isoquinolin-2-yl)-1-(1-methyl-1H-pyrazol-3-ylmethyl)-2-oxo-ethyl]-N-(4-pyridin-2-yl-benzyl)-3-(4-trifluoromethyl-phenyl)-acrylamide;
N-[2-(3,4-Dihydro-1H-isoquinolin-2-yl)-1-(1-methyl-1H-pyrazol-3-ylmethyl)-2-oxo-ethyl]-N-(4-pyridin-4-yl-benzyl)-3-(4-trifluoromethyl-phenyl)-acrylamide;
N-[2-(3,4-Dihydro-1H-isoquinolin-2-yl)-1-(2-methyl-2H-pyrazol-3-ylmethyl)-2-oxo-ethyl]-N-(4-pyridin-2-yl-benzyl)-3-(4-trifluoromethyl-phenyl)-acrylamide;
N-[2-(3,4-Dihydro-1H-isoquinolin-2-yl)-1-(2-methyl-2H-pyrazol-3-ylmethyl)-2-oxo-ethyl]-N-(4-pyridin-4-yl-benzyl)-3-(4-trifluoromethyl-phenyl)-acrylamide;
N-[2-(3,4-Dihydro-1H-isoquinolin-2-yl)-2-oxo-1-pyrimidin-2-ylmethyl-ethyl]-N-(4-pyridin-2-yl-benzyl)-3-(4-trifluoromethyl-phenyl)-acrylamide;
N-[2-(3,4-Dihydro-1H-isoquinolin-2-yl)-2-oxo-1-pyrimidin-2-ylmethyl-ethyl]-N-(4-pyridin-4-yl-benzyl)-3-(4-trifluoromethyl-phenyl)-acrylamide; and
N-[2-(3,4-Dihydro-1H-isoquinolin-2-yl)-1-isoxazol-3-ylmethyl-2-oxo-ethyl]-N-(4-pyridin-4-yl-benzyl)-3-(4-trifluoromethyl-phenyl)-acrylamide;
or a salt thereof.

13. A pharmaceutical composition comprising a compound of claim 1, or a pharmaceutically acceptable salt thereof, and a pharmaceutically acceptable carrier material.

14. (canceled)

15. A method for the treatment and/or prevention of protozoal infections comprising administering a composition according to claim 13 to a patient in need thereof.

16. A method according to claim 15
wherein the protozoal infection is malaria.
